SEQUENCE LISTING (44 sequences)

SEQ ID NO: 1 - RNA, 58 nt, synthetic construct
  source 1..58: mol_type = other RNA
ataccagcttattcaattagcaacatgagggggatagagggggtgggttctctcggct

SEQ ID NO: 2 - RNA, 60 nt, synthetic construct
  source 1..60: mol_type = other RNA
atgcggccgccgaccagaatcatgcaagtgcgtaagatagtcgcgggtcggcggccgcat

SEQ ID NO: 3 - RNA, 104 nt, synthetic construct
  source 1..104: mol_type = other RNA
agagcggccgctttttcagcaagattaagcccagggcagagccatctattgcttacatttgcttctgacacaactgtgttcactagcaacctcaaacagacacc

SEQ ID NO: 4 - RNA, 266 nt, synthetic construct
  source 1..266: mol_type = other RNA
agctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgc

SEQ ID NO: 5 - RNA, 321 nt, synthetic construct
  source 1..321: mol_type = other RNA
atgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcat

SEQ ID NO: 6 - RNA, 37 nt, synthetic construct
  source 1..37: mol_type = other RNA
cgaccagaatcatgcaagtgcgtaagatagtcgcggg

SEQ ID NO: 7 - RNA, 111 nt, synthetic construct
  source 1..111: mol_type = other RNA
gcccggatagctcagtcggtagagcagcggcctcgaccagaatcatgcaagtgcgtaagatagtcgcgggtcgaggccgcgtccagggttcaagtccctgttcgggcgcca

SEQ ID NO: 8 - DNA, 111 bp, synthetic construct
  source 1..111: mol_type = other DNA
gcccggatagctcagtcggtagagcagcggcctcgaccagaatcatgcaagtgcgtaagatagtcgcgggtcgaggccgcgtccagggttcaagtccctgttcgggcgcca

SEQ ID NO: 9 - DNA, 3172 bp, synthetic construct
  source 1..3172: mol_type = other DNA
taatacgactcactataggagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 3520 bp, synthetic construct
  source 1..3520: mol_type = other DNA
taatacgactcactataggggatccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaagcttagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 3520 bp, synthetic construct
  source 1..3520: mol_type = other DNA
taatacgactcactataggagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaggatccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaagcttagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 3520 bp, synthetic construct
  source 1..3520: mol_type = other DNA
taatacgactcactataggagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcggatccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaagcttagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 13 - DNA, 3520 bp, synthetic construct
  source 1..3520: mol_type = other DNA
taatacgactcactataggagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggatccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaagcttaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 3520 bp, synthetic construct
  source 1..3520: mol_type = other DNA
taatacgactcactataggagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaggatccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaagcttgaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 3259 bp, synthetic construct
  source 1..3259: mol_type = other DNA
taatacgactcactataggagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggatccgtagaaaatgcggccgccgaccagaatcatgcaagtgcgtaagatagtcgcgggtcggcggccgcatctgctgggaagcttaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 3346 bp, synthetic construct
  source 1..3346: mol_type = other DNA
taatacgactcactataggagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggatccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaagcttaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 3433 bp, synthetic construct
  source 1..3433: mol_type = other DNA
taatacgactcactataggagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggatccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagcttaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 18 - DNA, 3607 bp, synthetic construct
  source 1..3607: mol_type = other DNA
taatacgactcactataggagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggatccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagcttaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 19 - DNA, 3694 bp, synthetic construct
  source 1..3694: mol_type = other DNA
taatacgactcactataggagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggatccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaagcttaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 20 - DNA, 3349 bp, synthetic construct
  source 1..3349: mol_type = other DNA
taatacgactcactataggagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggatccaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaaagcttaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - DNA, 4472 bp, synthetic construct
  source 1..4472: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattctagaatttaggtgacactataggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaaactattattgctctgtcttacatcctgtgcctggtcttcgcccagaaaatccccggaaacgacaactctaccgccaccctgtgtctgggccaccacgccgtgccaaatggcaccatcgtgaagaccatcacaaacgacagaatcgaggtgaccaatgccacagagctggtgcagaacagctccatcggcgagatctgcgacagcccccaccagatcctggatggcgagaactgtacactgatcgacgccctgctgggcgaccctcagtgcgatggcttccagaataagaagtgggatctgtttgtggagagaagcaaggcctactccaactgttacccctatgacgtgcctgattatgcctctctgaggagcctggtggcctctagcggcaccctggagttcaagaacgagtcttttaattggaccggcgtgacacagaatggcacatcctctgcctgcatccgcggcagctcctctagcttctttagccggctgaactggctgacccacctgaattacacatatcctgccctgaacgtgaccatgccaaataaggagcagttcgataagctgtacatctggggagtgcaccacccaggaacagacaaggatcagatctttctgtatgcccagtcctctggcagaatcaccgtgtctacaaagaggagccagcaggccgtgatccctaacatcggctcccggccaagaatcagggacatcccctcccgcatctctatctactggaccatcgtgaagccaggcgatatcctgctgatcaactctacaggcaatctgatcgccccccgcggctatttcaagatccggagcggcaagagcagcatcatgcggtccgacgcccccatcggcaagtgcaagtctgagtgtatcacccctaacggcagcatcccaaatgataagccctttcagaacgtgaatcgcatcacatacggcgcctgtcctagatatgtgaagcacagcaccctgaagctggccacaggcatgagaaatgtgccagagaagcagaccaggggaatcttcggagcaatcgcaggctttatcgagaatggctgggagggcatggtggacggctggtacggcttccgccaccagaactccgagggaaggggacaggccgccgacctgaagtctacccaggcagccatcgatcagatcaacggcaagctgaatcgcctgatcggcaagacaaacgagaagttccaccagatcgagaaggagttttccgaggtggagggaagggtgcaggatctggagaagtacgtggaggacaccaagatcgatctgtggagctataatgccgagctgctggtggccctggagaaccagcacaccatcgacctgacagattccgagatgaataagctgttcgagaagaccaagaagcagctgagagagaacgccgaggacatgggcaatggctgctttaagatctaccacaagtgcgataacgcctgtatcgagagcatcaggaacgagacatacgaccacaacgtgtacagagatgaggccctgaacaataggtttcagatcaagggcgtggagctgaagtccggctataaggactggatcctgtggatctccttcgccatctcttgctttctgctgtgcgtggccctgctggggttcattatgtgggcttgtcagaaaggaaacattcgctgtaacatttgtatctaacgggtggcatccctgtgacccctccccagtgcctctcctggccctggaagttgccactccagtgcccaccagccttgtcctaataaaattaagttgcatcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 22 - DNA, 4502 bp, synthetic construct
  source 1..4502: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattctagaatttaggtgacactataggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaaactattattgctctgtcttacatcctgtgcctggtcttcgcccagaaaatccccggaaacgacaactctaccgccaccctgtgtctgggccaccacgccgtgccaaatggcaccatcgtgaagaccatcacaaacgacagaatcgaggtgaccaatgccacagagctggtgcagaacagctccatcggcgagatctgcgacagcccccaccagatcctggatggcgagaactgtacactgatcgacgccctgctgggcgaccctcagtgcgatggcttccagaataagaagtgggatctgtttgtggagagaagcaaggcctactccaactgttacccctatgacgtgcctgattatgcctctctgaggagcctggtggcctctagcggcaccctggagttcaagaacgagtcttttaattggaccggcgtgacacagaatggcacatcctctgcctgcatccgcggcagctcctctagcttctttagccggctgaactggctgacccacctgaattacacatatcctgccctgaacgtgaccatgccaaataaggagcagttcgataagctgtacatctggggagtgcaccacccaggaacagacaaggatcagatctttctgtatgcccagtcctctggcagaatcaccgtgtctacaaagaggagccagcaggccgtgatccctaacatcggctcccggccaagaatcagggacatcccctcccgcatctctatctactggaccatcgtgaagccaggcgatatcctgctgatcaactctacaggcaatctgatcgccccccgcggctatttcaagatccggagcggcaagagcagcatcatgcggtccgacgcccccatcggcaagtgcaagtctgagtgtatcacccctaacggcagcatcccaaatgataagccctttcagaacgtgaatcgcatcacatacggcgcctgtcctagatatgtgaagcacagcaccctgaagctggccacaggcatgagaaatgtgccagagaagcagaccaggggaatcttcggagcaatcgcaggctttatcgagaatggctgggagggcatggtggacggctggtacggcttccgccaccagaactccgagggaaggggacaggccgccgacctgaagtctacccaggcagccatcgatcagatcaacggcaagctgaatcgcctgatcggcaagacaaacgagaagttccaccagatcgagaaggagttttccgaggtggagggaagggtgcaggatctggagaagtacgtggaggacaccaagatcgatctgtggagctataatgccgagctgctggtggccctggagaaccagcacaccatcgacctgacagattccgagatgaataagctgttcgagaagaccaagaagcagctgagagagaacgccgaggacatgggcaatggctgctttaagatctaccacaagtgcgataacgcctgtatcgagagcatcaggaacgagacatacgaccacaacgtgtacagagatgaggccctgaacaataggtttcagatcaagggcgtggagctgaagtccggctataaggactggatcctgtggatctccttcgccatctcttgctttctgctgtgcgtggccctgctggggttcattatgtgggcttgtcagaaaggaaacattcgctgtaacatttgtatctaacgggtggcatccctgtgacccctccccagtgcctctcctggccctggaagttgccactccagtgcccaccagccttgtcctaataaaattaagttgcatcaaaaaaaaaaaaaaaaaaaaaaaaaaaaaaaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 23 - DNA, 4664 bp, synthetic construct
  source 1..4664: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattctagaatttaggtgacactataggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaaactattattgctctgtcttacatcctgtgcctggtcttcgcccagaaaatccccggaaacgacaactctaccgccaccctgtgtctgggccaccacgccgtgccaaatggcaccatcgtgaagaccatcacaaacgacagaatcgaggtgaccaatgccacagagctggtgcagaacagctccatcggcgagatctgcgacagcccccaccagatcctggatggcgagaactgtacactgatcgacgccctgctgggcgaccctcagtgcgatggcttccagaataagaagtgggatctgtttgtggagagaagcaaggcctactccaactgttacccctatgacgtgcctgattatgcctctctgaggagcctggtggcctctagcggcaccctggagttcaagaacgagtcttttaattggaccggcgtgacacagaatggcacatcctctgcctgcatccgcggcagctcctctagcttctttagccggctgaactggctgacccacctgaattacacatatcctgccctgaacgtgaccatgccaaataaggagcagttcgataagctgtacatctggggagtgcaccacccaggaacagacaaggatcagatctttctgtatgcccagtcctctggcagaatcaccgtgtctacaaagaggagccagcaggccgtgatccctaacatcggctcccggccaagaatcagggacatcccctcccgcatctctatctactggaccatcgtgaagccaggcgatatcctgctgatcaactctacaggcaatctgatcgccccccgcggctatttcaagatccggagcggcaagagcagcatcatgcggtccgacgcccccatcggcaagtgcaagtctgagtgtatcacccctaacggcagcatcccaaatgataagccctttcagaacgtgaatcgcatcacatacggcgcctgtcctagatatgtgaagcacagcaccctgaagctggccacaggcatgagaaatgtgccagagaagcagaccaggggaatcttcggagcaatcgcaggctttatcgagaatggctgggagggcatggtggacggctggtacggcttccgccaccagaactccgagggaaggggacaggccgccgacctgaagtctacccaggcagccatcgatcagatcaacggcaagctgaatcgcctgatcggcaagacaaacgagaagttccaccagatcgagaaggagttttccgaggtggagggaagggtgcaggatctggagaagtacgtggaggacaccaagatcgatctgtggagctataatgccgagctgctggtggccctggagaaccagcacaccatcgacctgacagattccgagatgaataagctgttcgagaagaccaagaagcagctgagagagaacgccgaggacatgggcaatggctgctttaagatctaccacaagtgcgataacgcctgtatcgagagcatcaggaacgagacatacgaccacaacgtgtacagagatgaggccctgaacaataggtttcagatcaagggcgtggagctgaagtccggctataaggactggatcctgtggatctccttcgccatctcttgctttctgctgtgcgtggccctgctggggttcattatgtgggcttgtcagaaaggaaacattcgctgtaacatttgtatctaacgggtggcatccctgtgacccctccccagtgcctctcctggccctggaagttgccactccagtgcccaccagccttgtcctaataaaattaagttgcatcgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaaaaaaaaaaaaaaaaaaaaaaaaaaaaaaaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 24 - DNA, 4838 bp, synthetic construct
  source 1..4838: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattctagaatttaggtgacactataggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaaactattattgctctgtcttacatcctgtgcctggtcttcgcccagaaaatccccggaaacgacaactctaccgccaccctgtgtctgggccaccacgccgtgccaaatggcaccatcgtgaagaccatcacaaacgacagaatcgaggtgaccaatgccacagagctggtgcagaacagctccatcggcgagatctgcgacagcccccaccagatcctggatggcgagaactgtacactgatcgacgccctgctgggcgaccctcagtgcgatggcttccagaataagaagtgggatctgtttgtggagagaagcaaggcctactccaactgttacccctatgacgtgcctgattatgcctctctgaggagcctggtggcctctagcggcaccctggagttcaagaacgagtcttttaattggaccggcgtgacacagaatggcacatcctctgcctgcatccgcggcagctcctctagcttctttagccggctgaactggctgacccacctgaattacacatatcctgccctgaacgtgaccatgccaaataaggagcagttcgataagctgtacatctggggagtgcaccacccaggaacagacaaggatcagatctttctgtatgcccagtcctctggcagaatcaccgtgtctacaaagaggagccagcaggccgtgatccctaacatcggctcccggccaagaatcagggacatcccctcccgcatctctatctactggaccatcgtgaagccaggcgatatcctgctgatcaactctacaggcaatctgatcgccccccgcggctatttcaagatccggagcggcaagagcagcatcatgcggtccgacgcccccatcggcaagtgcaagtctgagtgtatcacccctaacggcagcatcccaaatgataagccctttcagaacgtgaatcgcatcacatacggcgcctgtcctagatatgtgaagcacagcaccctgaagctggccacaggcatgagaaatgtgccagagaagcagaccaggggaatcttcggagcaatcgcaggctttatcgagaatggctgggagggcatggtggacggctggtacggcttccgccaccagaactccgagggaaggggacaggccgccgacctgaagtctacccaggcagccatcgatcagatcaacggcaagctgaatcgcctgatcggcaagacaaacgagaagttccaccagatcgagaaggagttttccgaggtggagggaagggtgcaggatctggagaagtacgtggaggacaccaagatcgatctgtggagctataatgccgagctgctggtggccctggagaaccagcacaccatcgacctgacagattccgagatgaataagctgttcgagaagaccaagaagcagctgagagagaacgccgaggacatgggcaatggctgctttaagatctaccacaagtgcgataacgcctgtatcgagagcatcaggaacgagacatacgaccacaacgtgtacagagatgaggccctgaacaataggtttcagatcaagggcgtggagctgaagtccggctataaggactggatcctgtggatctccttcgccatctcttgctttctgctgtgcgtggccctgctggggttcattatgtgggcttgtcagaaaggaaacattcgctgtaacatttgtatctaacgggtggcatccctgtgacccctccccagtgcctctcctggccctggaagttgccactccagtgcccaccagccttgtcctaataaaattaagttgcatcgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggtagctgtgaaccgtagaaaatgcggccgccgaccagaatcatgcaagtgcgtaagatagtcgcgggtcggcggccgcatctgctgggaagctacgatccgtagaaaatgcggccgccgaccagaatcatgcaagtgcgtaagatagtcgcgggtcggcggccgcatctgctgggaaaaaaaaaaaaaaaaaaaaaaaaaaaaaaaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 25 - DNA, 4667 bp, synthetic construct
  source 1..4667: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattctagaatttaggtgacactataggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaaactattattgctctgtcttacatcctgtgcctggtcttcgcccagaaaatccccggaaacgacaactctaccgccaccctgtgtctgggccaccacgccgtgccaaatggcaccatcgtgaagaccatcacaaacgacagaatcgaggtgaccaatgccacagagctggtgcagaacagctccatcggcgagatctgcgacagcccccaccagatcctggatggcgagaactgtacactgatcgacgccctgctgggcgaccctcagtgcgatggcttccagaataagaagtgggatctgtttgtggagagaagcaaggcctactccaactgttacccctatgacgtgcctgattatgcctctctgaggagcctggtggcctctagcggcaccctggagttcaagaacgagtcttttaattggaccggcgtgacacagaatggcacatcctctgcctgcatccgcggcagctcctctagcttctttagccggctgaactggctgacccacctgaattacacatatcctgccctgaacgtgaccatgccaaataaggagcagttcgataagctgtacatctggggagtgcaccacccaggaacagacaaggatcagatctttctgtatgcccagtcctctggcagaatcaccgtgtctacaaagaggagccagcaggccgtgatccctaacatcggctcccggccaagaatcagggacatcccctcccgcatctctatctactggaccatcgtgaagccaggcgatatcctgctgatcaactctacaggcaatctgatcgccccccgcggctatttcaagatccggagcggcaagagcagcatcatgcggtccgacgcccccatcggcaagtgcaagtctgagtgtatcacccctaacggcagcatcccaaatgataagccctttcagaacgtgaatcgcatcacatacggcgcctgtcctagatatgtgaagcacagcaccctgaagctggccacaggcatgagaaatgtgccagagaagcagaccaggggaatcttcggagcaatcgcaggctttatcgagaatggctgggagggcatggtggacggctggtacggcttccgccaccagaactccgagggaaggggacaggccgccgacctgaagtctacccaggcagccatcgatcagatcaacggcaagctgaatcgcctgatcggcaagacaaacgagaagttccaccagatcgagaaggagttttccgaggtggagggaagggtgcaggatctggagaagtacgtggaggacaccaagatcgatctgtggagctataatgccgagctgctggtggccctggagaaccagcacaccatcgacctgacagattccgagatgaataagctgttcgagaagaccaagaagcagctgagagagaacgccgaggacatgggcaatggctgctttaagatctaccacaagtgcgataacgcctgtatcgagagcatcaggaacgagacatacgaccacaacgtgtacagagatgaggccctgaacaataggtttcagatcaagggcgtggagctgaagtccggctataaggactggatcctgtggatctccttcgccatctcttgctttctgctgtgcgtggccctgctggggttcattatgtgggcttgtcagaaaggaaacattcgctgtaacatttgtatctaacgggtggcatccctgtgacccctccccagtgcctctcctggccctggaagttgccactccagtgcccaccagccttgtcctaataaaattaagttgcatcaaaaaaaaaaaaagcccggatagctcagtcggtagagcagcggcctatgcggccgccgaccagaatcatgcaagtgcgtaagatagtcgcgggtcggcggccgcattcgaggccgcgtccagggttcaagtccctgttcgggcgccactgcagaaaaaaaaaaaaaaaaaaaaaaaaaaaaaaaaaaaaaaaaaaaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 26 - DNA, 3301 bp, synthetic construct
  source 1..3301: mol_type = other DNA
atttaggtgacactatagagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggatccttgccatgtgtatgtgggatgcggccgccgaccagaatcatgcaagtgcgtaagatagtcgcgggtcggcggccgcatcccacatactctgatgatccttcgggatcattcatggcaaaagcttaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 27 - DNA, 3357 bp, synthetic construct
  source 1..3357: mol_type = other DNA
atttaggtgacactatagagagcggccgctttttcagcaagattaagcccagggcagagccatctattgcttacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggatccttgccatgtgtatgtgggatgcggccgccgaccagaatcatgcaagtgcgtaagatagtcgcgggtcggcggccgcatcccacatactctgatgatccatgcggccgccgaccagaatcatgcaagtgcgtaagatagtcgcgggtcggcggccgcatggatcattcatggcaaaagcttaaaaaaaaaaaaaaaaaaaaaaaaaaaaaagaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 28 - DNA, 118 bp, synthetic construct
  source 1..118: mol_type = other DNA
ttgccatgtgtatgtgggatgcggccgccgaccagaatcatgcaagtgcgtaagatagtcgcgggtcggcggccgcatcccacatactctgatgatccttcgggatcattcatggcaa

SEQ ID NO: 29 - RNA, 118 nt, synthetic construct
  source 1..118: mol_type = other RNA
ttgccatgtgtatgtgggatgcggccgccgaccagaatcatgcaagtgcgtaagatagtcgcgggtcggcggccgcatcccacatactctgatgatccttcgggatcattcatggcaa

SEQ ID NO: 30 - DNA, 174 bp, synthetic construct
  source 1..174: mol_type = other DNA
ttgccatgtgtatgtgggatgcggccgccgaccagaatcatgcaagtgcgtaagatagtcgcgggtcggcggccgcatcccacatactctgatgatccatgcggccgccgaccagaatcatgcaagtgcgtaagatagtcgcgggtcggcggccgcatggatcattcatggcaa

SEQ ID NO: 31 - RNA, 174 nt, synthetic construct
  source 1..174: mol_type = other RNA
ttgccatgtgtatgtgggatgcggccgccgaccagaatcatgcaagtgcgtaagatagtcgcgggtcggcggccgcatcccacatactctgatgatccatgcggccgccgaccagaatcatgcaagtgcgtaagatagtcgcgggtcggcggccgcatggatcattcatggcaa

SEQ ID NO: 32 - DNA, 18 bp, synthetic construct
  source 1..18: mol_type = other DNA
ttgccatgtgtatgtggg

SEQ ID NO: 33 - DNA, 40 bp, synthetic construct
  source 1..40: mol_type = other DNA
cccacatactctgatgatccttcgggatcattcatggcaa

SEQ ID NO: 34 - RNA, 18 nt, synthetic construct
  source 1..18: mol_type = other RNA
ttgccatgtgtatgtggg

SEQ ID NO: 35 - RNA, 40 nt, synthetic construct
  source 1..40: mol_type = other RNA
cccacatactctgatgatccttcgggatcattcatggcaa

SEQ ID NO: 36 - DNA, 18 bp, synthetic construct
  source 1..18: mol_type = other DNA
ttgccatgtgtatgtggg

SEQ ID NO: 37 - DNA, 20 bp, synthetic construct
  source 1..20: mol_type = other DNA
cccacatactctgatgatcc

SEQ ID NO: 38 - DNA, 16 bp, synthetic construct
  source 1..16: mol_type = other DNA
ggatcattcatggcaa

SEQ ID NO: 39 - RNA, 18 nt, synthetic construct
  source 1..18: mol_type = other RNA
ttgccatgtgtatgtggg

SEQ ID NO: 40 - RNA, 20 nt, synthetic construct
  source 1..20: mol_type = other RNA
cccacatactctgatgatcc

SEQ ID NO: 41 - RNA, 16 nt, synthetic construct
  source 1..16: mol_type = other RNA
ggatcattcatggcaa

SEQ ID NO: 42 - DNA, 111 bp, synthetic construct; identical to SEQ ID NO: 8
  source 1..111: mol_type = other DNA

SEQ ID NO: 43 - RNA, 204 nt, synthetic construct
  source 1..204: mol_type = other RNA
gaacgaaactctgggagctgcgattggcagaattccgttagcaaggccgcaggacttgcatgcttatcctgcggcgcgggcgcgtttcccgggttacgcgcccgccttaagtgtttctcgagttggcacttaagcttgctaacggaattcccccatatccaacttccaatttaatctttcttttttaattttcacttatttgcg

SEQ ID NO: 44 - DNA, 204 bp, synthetic construct
  source 1..204: mol_type = other DNA
gaacgaaactctgggagctgcgattggcagaattccgttagcaaggccgcaggacttgcatgcttatcctgcggcgcgggcgcgtttcccgggttacgcgcccgccttaagtgtttctcgagttggcacttaagcttgctaacggaattcccccatatccaacttccaatttaatctttcttttttaattttcacttatttgcg

(19) United States
(12) Patent Application Publication
(10) Pub. No.: US 2024/0327847 A1
(43) Pub. Date: Oct. 3, 2024

(54) COMPOSITIONS AND METHODS FOR RNA AFFINITY

(71) Applicant: SANOFI, Paris (FR)

(72) Inventors: Jianping CUI, Cambridge, MA (US); Tong-Ming FU, Cambridge, MA (US); Allison Jessica MATTHEWS, Cambridge, MA (US)

(21) Appl. No.: 18/586,102

(22) Filed: Feb. 23, 2024

Related U.S. Application Data
(63) Continuation of application No. PCT/IB2022/058234, filed on Sep. 1, 2022.
(60) Provisional application No. 63/240,027, filed on Sep. 2, 2021.

(30) Foreign Application Priority Data
Jul. 20, 2022 (EP) 22315159.8

(51) Int. Cl. C12N 15/115; A61K 48/00; C12N 15/10

(52) U.S. Cl. CPC C12N 15/115; A61K 48/0066; C12N 15/101; C12N 2310/16; C12N 2310/351; C12N 2830/50

(57) ABSTRACT

The present disclosure provides methods and compositions for RNA affinity purification. In particular, the disclosure relates to compositions and methods of making and using mRNA comprising one or more aptamers which specifically bind an affinity ligand.

### RELATED APPLICATIONS

This application is a continuation of International Patent Application No. PCT/IB2022/058234, filed Sep. 1, 2022, which claims the benefit of priority of U.S. Provisional Patent Application Ser. No. 63/240,027, filed Sep. 2, 2021, and EP Priority application Ser. No. 22/315,159.8, filed Jul. 20, 2022, the content of each is incorporated by reference in their entirety for all purposes.

### SEQUENCE LISTING

The instant application contains a Sequence Listing which has been submitted electronically in XML format and is hereby incorporated by reference in its entirety. Said XML file, created on Feb. 23, 2024, is named 750045_SA9-312PCCON_ST26.xml and is 112,573 bytes in size.

### BACKGROUND OF THE DISCLOSURE

Messenger RNA (mRNA) therapeutics are becoming an increasingly important approach for the treatment of a variety of diseases and is an emerging alternative to protein replacement therapies, antibody therapies, conventional vaccine therapies, and/or gene therapies. In a mRNA therapeutic, the mRNA encoding the protein or peptide of interest is delivered to the patient or the target cell of the patient. Upon entry of the mRNA into the patient's target cell, the patient's translational machinery produces and subsequently express the protein or peptide of interest. Thus, it is important to ensure the production of highly pure and safe mRNA product.

mRNA for therapeutics are often synthesized using in vitro transcription systems with enzymes such as RNA polymerases transcribing mRNA from template plasmid DNA, along with or followed by addition of a 5′-cap and 3′-polyadenylation. The result of such reactions is a composition which includes full-length mRNA and various undesirable contaminants, e.g., proteins, non-RNA nucleic acids, undesired RNA species, spermidine, DNA, pyrophosphates, endotoxins, detergents, and organic solvents. These contaminants must be purified to provide a clean and homogeneous mRNA that is suitable for therapeutic use.

There remains a need for more effective, reliable, and safer methods of purifying RNA from large scale manufacturing processes for potential therapeutic applications.

### BRIEF SUMMARY OF THE DISCLOSURE

From the description herein, it will be appreciated that that the present disclosure encompasses multiple aspects and embodiments which include, but are not limited to, the following:

In one aspect, the disclosure provides a messenger RNA (mRNA) comprising at least one 5′ untranslated region (5′ UTR), at least one open reading frame (ORF), at least one 3′ untranslated region (3′ UTR), and at least one polyadenylation (polyA) sequence, wherein the mRNA comprises at least one RNA aptamer.

In some embodiments, the RNA aptamer is embedded in an RNA scaffold.

In some embodiments, the RNA scaffold comprises at least one secondary structure motif. In some embodiments, the secondary structure motif is a tetraloop, a pseudoknot, or a stem-loop. In some embodiments, the RNA scaffold comprises at least one tertiary structure. In some embodiments the secondary structure motif and/or tertiary structure are nuclease resistant.

In some embodiments, the RNA scaffold is a transfer RNA (tRNA), a ribosomal RNA (rRNA), or a ribozyme. In some embodiments, the ribozyme is catalytically inactive. In some embodiments, the RNA scaffold comprises a transfer RNA (tRNA). In some embodiments, the RNA aptamer is embedded in a tRNA hairpin loop of the tRNA. In some embodiments, the RNA aptamer is embedded in a tRNA anticodon loop of the tRNA. In some embodiments, the RNA aptamer is embedded in a tRNA D loop of the tRNA. In some embodiments, the RNA aptamer is embedded in a tRNA T loop of the tRNA.

In some embodiments, the RNA aptamer is positioned in the 5′ UTR. In some embodiments, the RNA aptamer is positioned between the 3′ end of the ORF and the 5′ end of the 3′ UTR. In some embodiments, the RNA aptamer is positioned in the 3′ UTR. In some embodiments, the RNA aptamer is positioned between the 3′ end of the 3′UTR and the 5′ end of the polyA sequence. In some embodiments, wherein the RNA aptamer is positioned at the 3′ end of the polyA sequence.

In some embodiments, the mRNA comprises or consists of one RNA aptamer. In some embodiments, the mRNA comprises between one and four RNA aptamers. In some embodiments, the RNA aptamers are identical. In some embodiments, the RNA aptamers are distinct.

In some embodiments, the RNA aptamer is synthetically derived. In some embodiments, the RNA aptamer is a split aptamer or an X-aptamer. In some embodiments, the RNA aptamer is naturally-derived. In some embodiments, the RNA aptamer is derived from a hairpin RNA, a tRNA, or a riboswitch.

In some embodiments, the RNA aptamer embedded in a bioorthogonal scaffold.

In some embodiments, the bioorthogonal scaffold is V5, F29, F30, or a variant thereof.

In some embodiments, the bioorthogonal scaffold comprises a 5′ nucleotide sequence of SEQ ID NO: 34 and a 3′ nucleotide sequence of SEQ ID NO: 35, wherein an aptamer sequence is positioned between SEQ ID NO: 34 and SEQ ID NO: 35.

In some embodiments, the bioorthogonal scaffold comprises a 5′ nucleotide sequence of SEQ ID NO: 39, an internal nucleotide sequence of SEQ ID NO: 40, and a 3′ nucleotide sequence of SEQ ID NO: 41, wherein a first aptamer sequence is positioned between SEQ ID NO: 39 and SEQ ID NO: 40 and a second aptamer sequence is positioned between SEQ ID NO: 40 and SEQ ID NO: 41, optionally wherein the first and second aptamer are the same or different.

In some embodiments, the RNA aptamer embedded bioorthogonal scaffold comprises the nucleotide sequence of SEQ ID NO: 29 or SEQ ID NO: 31.

In some embodiments, the RNA aptamer binds to an affinity ligand. In some embodiments, the affinity ligand comprises protein A, protein G, streptavidin, glutathione, dextran, or a fluorescent molecule. In some embodiments, the affinity ligand comprises streptavidin. In some embodiments, the affinity ligand is immobilized on a chromatography resin.

In some embodiments, the RNA aptamer is Sim or Sm. In some embodiments, the mRNA comprises between one and four S1m or sm RNA aptamers. In some embodiments, the S1m or sm RNA aptamer is positioned: 1) between the 3′ end of the ORF and the 5′ end of the 3′ UTR; 2) in the 3′ UTR; 3) between the 3′ end of the 3′UTR and the 5′ end of the polyA sequence; and/or; 4) at the 3′ end of the polyA sequence. In some embodiments, the RNA aptamer comprises the nucleotide sequence of SEQ ID NO: 2 or 6. In some embodiments, the RNA aptamer embedded tRNA comprises the nucleotide sequence of SEQ ID NO: 7.

In some embodiments, the mRNA encodes at least one polypeptide. In some embodiments, the polypeptide is a biologically active polypeptide, a therapeutic polypeptide, or an antigenic polypeptide. In some embodiments, the antigenic polypeptide comprises an antibody or fragment thereof, enzyme replacement polypeptide, or genome-editing polypeptide. In some embodiments, the therapeutic polypeptide comprises an antibody heavy chain, an antibody light chain, an enzyme, or a cytokine. In some embodiments, the biologically active polypeptide comprises a genome-editing polypeptide.

In some embodiments, the mRNA contains a chimeric 5′ or 3′ UTR.

In some embodiments, the mRNA comprises at least one chemical modification. In some embodiments, the chemical modification is pseudouridine, N1-methylpseudouridine, 2-thiouridine, 4′-thiouridine, 5-methylcytosine, 2-thio-I-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-I-methyl-pseudouridine, 4-thio-pseudouridine, 5-aza-uridine, dihydropseudouridine, 5-methyluridine, 5-methyluridine, 5-methoxyuridine, or 2′-O-methyl uridine. In some embodiments, the chemical modification is pseudouridine, N1-methylpseudouridine, 5-methylcytosine, 5-methoxyuridine, or a combination thereof. In some embodiments, the chemical modification is N1-methylpseudouridine.

In some embodiments, the polyA sequence is at least 10 consecutive adenosine residues. In some embodiments, the polyA sequence is between 10 and 500 consecutive adenosine residues. In some embodiments, the mRNA comprises two polyA sequences, each polyA sequence comprising between 10 and 500 consecutive adenosine residues, wherein at least one RNA aptamer or RNA aptamer embedded tRNA is positioned between the two polyA sequences.

In some embodiments, the mRNA comprises a 5′ cap.

In some embodiments, the translation efficiency of the mRNA is substantially the same compared to an mRNA that does not comprise an RNA aptamer.

In some embodiments, the mRNA is synthesized using in vitro transcription (IVT).

In some embodiments, the mRNA is expressed in vivo or ex vivo.

In one aspect, the disclosure provides a vector encoding the mRNA described above. In some embodiments, the vector comprises at least elements a-e, from 5′ to 3′: a) an RNA polymerase promoter; b) a polynucleotide sequence encoding a 5′ UTR; c) a polynucleotide sequence encoding an ORF; d) a polynucleotide sequence encoding a 3′ UTR; and e) a polynucleotide sequence encoding at least one RNA aptamer. In some embodiments, the vector further comprises a polynucleotide sequence encoding a polyA sequence and/or a polyadenylation signal.

In another aspect, the disclosure provides a host cell comprising the vector described above.

In another aspect, the disclosure provides a pharmaceutical composition comprising the mRNA described above. In some embodiments, the pharmaceutical composition is administered to a subject in need thereof in a method of treating or preventing a disease or disorder.

In another aspect, disclosed herein is a method for purifying an mRNA, comprising the steps of: (a) contacting a sample comprising the mRNA with an affinity ligand that is immobilized on a chromatography resin, wherein the RNA aptamer comprises binding affinity for the affinity ligand; (b) eluting the mRNA from the chromatography resin; and (c) purifying the mRNA from the sample. In some embodiments, the method comprises one or more washing steps between the contacting step (a) and the eluting step (b).

In another aspect, disclosed herein is a method of purifying an RNA, comprising the steps of: (a) contacting a sample comprising the RNA with an affinity ligand that is immobilized on a chromatography resin; (b) eluting the RNA from the chromatography resin; and (c) isolating the RNA from the sample, wherein the RNA comprises at least one open reading frame (ORF) and at least one RNA aptamer, wherein the RNA aptamer comprises binding affinity for the affinity ligand.

In some embodiments, the RNA further comprises at least one 5′ untranslated region (5′ UTR), at least one 3′ untranslated region (3′ UTR), and at least one polyadenylation (polyA) sequence.

In some embodiments, the RNA is at least about 500 nucleotides in length, at least about 750 nucleotides in length, at least about 1,000 nucleotides in length, at least about 1,500 nucleotides in length, at least about 2,000 nucleotides in length, at least about 2,500 nucleotides in length, at least about 3,000 nucleotides in length, at least about 3,500 nucleotides in length, at least about 4,000 nucleotides in length, at least about 4,500 nucleotides in length, or at least about 5,000 nucleotides in length.

In some embodiments, the RNA comprises a 5′ cap. In some embodiments, the RNA is an mRNA.

In some embodiments, the mRNA is greater than or equal to 90% pure.

In another aspect, disclosed herein is a method for purifying an mRNA, comprising the steps of: (a) contacting a sample comprising the mRNA with an affinity ligand that is immobilized on a chromatography resin; (b) eluting the mRNA from the chromatography resin; and (c) isolating the mRNA from the sample, wherein the mRNA comprises at least one 5′ untranslated region (5′ UTR), at least one open reading frame (ORF), at least one 3′ untranslated region (3′ UTR), at least one polyadenylation (polyA) sequence, and at least one RNA aptamer, wherein the RNA aptamer comprises binding affinity for the affinity ligand. In some embodiments, the mRNA is greater than or equal to 90% pure.

In another aspect, disclosed herein is a pharmaceutical composition comprising a plurality of mRNA molecules, wherein at least about 90% of an mRNA comprise at least one 5′ untranslated region (5′ UTR), at least one open reading frame (ORF), at least one 3′ untranslated region (3′ UTR), at least one polyadenylation (polyA) sequence, and at least one RNA aptamer.

In another aspect, disclosed herein is a messenger RNA (mRNA) comprising at least one 5′ untranslated region (5′ UTR), at least one open reading frame (ORF), at least one 3′ untranslated region (3′ UTR), and at least one polyadenylation (polyA) sequence, wherein the mRNA comprises at least one tRNA.

In another aspect, disclosed herein is a messenger RNA (mRNA) comprising at least one 5′ untranslated region (5′ UTR), at least one open reading frame (ORF), at least one 3′ untranslated region (3′ UTR), and at least one polyadenylation (polyA) sequence, wherein the mRNA comprises at least one RNA aptamer embedded tRNA.

In another aspect, disclosed herein is a messenger RNA (mRNA) comprising at least one 5′ untranslated region (5′ UTR), at least one open reading frame (ORF), at least one 3′ untranslated region (3′ UTR), and at least one polyadenylation (polyA) sequence, wherein the mRNA comprises at least one RNA aptamer embedded biorthogonal scaffold.

### BRIEF DESCRIPTION OF THE DRAWINGS/FIGURES

The foregoing and other features and advantages of the present disclosure will be more fully understood from the following detailed description of illustrative embodiments taken in conjunction with the accompanying drawings.

FIG. 1 schematizes the steps in the aptamer tagged mRNA affinity purification process.

FIG. 2 shows the RNA concentration (ng) as measured on a Nanodrop prior to incubation with streptavidin sepharose beads (input) or following streptavidin affinity binding purification and elution steps with either a random aptamer or the S1m aptamer (unbound versus eluted). Percent RNA recovered after affinity purification is relative to the input sample that did not undergo affinity purification.

FIG. 3 depicts the plasmid maps of pAM14 (2,496 bp) carrying an ARE element tagged with the 4×S1m aptamer or the pAM15 plasmid (2,168 bp) carrying the untagged ARE element.

FIG. 4 shows the RNA concentration (ng) as measured on a Nanodrop prior to incubation with streptavidin sepharose beads (input) or following streptavidin affinity binding purification and elution steps with either a TNFα-53 tagged 4×S1m aptamer mRNA or a TNFα-53 mRNA negative control (unbound versus eluted). Percent RNA purified is relative to input sample that did not undergo affinity purification.

FIG. 5 depicts the following plasmid maps containing the following constructs: (1) pAM22, a control plasmid of 2,173 bp, carrying a M. thermautotrophicust tRNA^{GLN2} scaffold (pAM22 (tRNA); plasmid map annotates the position of the anticodon arms with respect to the Gln2 anticodon loop) (2) pAM20, a control plasmid of 2,134 bp, carrying a Sm aptamer (pAM20 (Sm)), (3) pAM21, an experimental plasmid of 2,206 bp, carrying the Sm aptamer sequence embedded in a portion of the anticodon loop tRNA^{GLN2} sequence which is flanked on both sides by the tRNA anticodon arm sequence (pAM21 (TRNA Sm), and (4) pAM23, an experimental plasmid of 2,306 bp, carrying tandem two-repeat configuration of the Sm-tRNA^{GLN2} construct (2× RNA Sm). Each tag was driven by a T7 promoter.

FIG. 6 shows the RNA concentration (ng) as measured on a Nanodrop prior to incubation with streptavidin sepharose beads (input) or following streptavidin affinity binding purification wash steps (wash 1-3) and elution step (eluted) with either mRNA containing the Sm, tRNA, tRNA-Sm, and 2× tRNA Sm aptamer tags. Percent RNA recovery after affinity purification is relative to the input sample that did not undergo affinity purification.

FIG. 7 illustrates the aptamer tagging strategies for optimized binding affinity and translation of mRNA based on aptamer-transcript localization, aptamer copy number, an aptamer embedded in a tRNA scaffold, and tandem repeat copies of an aptamer embedded in a tRNA scaffold.

FIG. 8 depicts plasmid maps pAM11 (3,541 bp) carrying humanized enhanced green fluorescent protein (hEGFP) and pAM8 plasmid (3,213 bp) carrying hEGFP tagged with a 4×S1m aptamer.

FIG. 9 is an image of an agarose gel containing mRNA generated from an IVT reaction of PCR product template for hEGFP (lane 1, derived from pAM11) and hEGFP tagged with 4×S1m aptamer (lane 2, derived from pAM8).

FIG. 10 shows the RNA concentration (ng) as measured on a Nanodrop prior to incubation with streptavidin sepharose beads (input) or following streptavidin affinity binding purification and elution step (eluted) with either mRNA containing the hEGFP or hEGFP tagged with a 4×S1m aptamer. Percent RNA purified is relative to input sample that did not undergo affinity purification.

FIG. 11 are representative fluorescent microscopy images taken of HEK293FT cells transfected with hEGFP or hEGFP-4×S1m mRNA after 24 hours.

FIG. 12 displays a panel of representative fluorescent microscopy images taken of HEK293FT cells transfected with hEGFP (left column, top panel), hEGFP-4×S1m (left column, bottom panel), hEGFP with longer polyA tail (right column, top panel), or hEGFP-4×S1m with longer polyA tail (right column, bottom panel) mRNA after 24 hours.

FIG. 13A-FIG. 13B tests whether the topological order of the S1m aptamer impacts downstream mRNA affinity purification. FIG. 13A is a schematic of the experimental constructs designed to test the S1m aptamer position in the mRNA transcript. The Sim aptamer was either placed (1) directly upstream of the 5′ UTR; (2) directly upstream of the 3′UTR; (3) in the 3′ UTR; (4) directly downstream the 3′ UTR; or (5) in the 3′ end of the polyA sequence. FIG. 13B shows the percent of RNA recovered after affinity purification relative to the input sample that did not undergo affinity purification following streptavidin binding and elution steps (unbound versus eluted).

FIG. 14 tests whether the aptamer copy number (valency) in the transcript impacts downstream mRNA affinity purification. FIG. 14 shows the percent of RNA recovered after affinity purification relative to the input sample that did not undergo affinity purification following streptavidin binding and elution steps (unbound versus eluted) with mRNA constructs that contained between one and six copies of S1m aptamer.

FIG. 15 shows the percent of RNA recovered after mRNA affinity purification relative to the input sample that did not undergo affinity purification following streptavidin binding and elution steps (unbound versus eluted) with 2×S1m, 4×S1m, or the tRNA Sim aptamer tagged mRNA containing a different protein-coding sequence (Singapore '16 hemagglutinin) and distinct UTRs.

FIG. 16A-FIG. 16C tests whether the aptamer placement in the mRNA transcript impacts translation kinetics in HSKMc cells. FIG. 16A is a schematic of the experimental constructs designed to test the impact of the Sim aptamer position relative to the other topologically ordered components of the mRNA. FIG. 16B is a bar graph of the total number of GFP positive cells (expressed as percent) as calculated by flow cytometry analysis for HSKMc cells transfected with either the untagged control mRNA or one of the five aptamer tagged mRNAs shown in FIG. 16A. FIG. 16C is a bar graph displaying the number of GFP positive high cells (expressed as percent) in FIG. 16B.

FIG. 17A-FIG. 17C tests whether the aptamer placement in the mRNA transcript impacts translation kinetics in Hela cells. FIG. 17A is a schematic of the experimental constructs designed to test the impact of the S1m aptamer position relative to the other topologically ordered components of the mRNA. FIG. 17B is a bar graph of the total number of GFP positive cells (expressed as percent) as calculated by flow cytometry analysis for Hela cells transfected with either the untagged control mRNA or one of the five aptamer tagged mRNAs shown in FIG. 17A. FIG. 17C is a bar graph displaying only the number of GFP positive high cells (expressed as percent) in FIG. 17B.

FIG. 18 depicts a bar graph of the total number of GFP positive cells (expressed as percent) as calculated by flow cytometry analysis for Hela cells transfected with either the controls or with an aptamer tagged mRNA which had increased polyA tail length (labeled, “Aptamer, poly (A) 2×60_6+A's”).

FIG. 19A-FIG. 19B examines whether the stabilization of an S1m aptamer with a tRNA scaffold impacts mRNA affinity purification and the subsequent mRNA translational efficiency. FIG. 19A is a bar graph which shows the percent of RNA recovered after mRNA affinity purification relative to the input sample following streptavidin binding and elution steps (unbound versus eluted) with the untagged mRNA control, the 2×S1m aptamer, the 4×S1m aptamer transcript, or RNA S1m aptamer tagged mRNA. FIG. 19B is a bar graph of the total number of GFP positive Hela cells (expressed as percent) as calculated by flow cytometry analysis after transfection with the untagged mRNA control or the tRNA S1m aptamer tagged mRNA (labeled, “tRNA stabilized aptamer”).

FIG. 20A is the secondary RNA structure formed by the F30-aptamer. FIG. 20B is a bar graph which shows the percent of RNA recovered after mRNA affinity purification relative to the input sample following streptavidin binding and elution steps (unbound versus eluted) with the untagged mRNA control, the 4×S1m aptamer, the 1×S1m aptamer stabilized in a F30 scaffold (F30-1×S1m), or the 2×S1m aptamer stabilized in a F30 scaffold (F30-2×S1m) tagged mRNA.

FIG. 20C shows the RNA concentration (ng) as measured on a Nanodrop prior to incubation with streptavidin sepharose beads (input) or following streptavidin affinity binding purification and elution step (eluted) with either the untagged mRNA control, the 4×S1m aptamer, the F30-2×S1m aptamer, or the F30-1×S1m tagged mRNA.

### DETAILED DESCRIPTION OF THE DISCLOSURE

The present disclosure is directed to, inter alia, novel mRNA compositions and methods for RNA affinity purification. In particular, the disclosure relates to mRNA compositions comprising at least one RNA aptamer. The RNA aptamers associated with the disclosed mRNA compositions enable the use of effective affinity purification methods yet have minimal impact on translation efficiency and immunogenicity. Also disclosed herein are methods of making these mRNA-tagged aptamer compositions.

### I. Definitions

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

Depending on context, the term “polynucleotide” or “nucleotide” may encompass a singular nucleic acid as well as plural nucleic acids. In some embodiments, a polynucleotide is an isolated nucleic acid molecule or construct, e.g., messenger RNA (mRNA) or plasmid DNA (pDNA). In some embodiments, a polynucleotide comprises a conventional phosphodiester bond. In some embodiments, a polynucleotide comprises a non-conventional bond (e.g., an amide bond, such as found in peptide nucleic acids (PNA)). The term “nucleic acid” may refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a Factor VIII polypeptide contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

An “isolated” polypeptide or a fragment, variant, or derivative thereof refers to a polypeptide that is not in its natural milieu. No particular level of purification is required. For example, an isolated polypeptide can simply be removed from its native or natural environment. Recombinantly produced polypeptides and proteins expressed in host cells are considered isolated for the purpose of the disclosure, as are native or recombinant polypeptides which have been separated, fractionated, or partially or substantially purified by any suitable technique.

“Administer” or “administering,” as used herein refers to delivering to a subject a composition described herein, e.g., a chimeric protein. The composition, e.g., the chimeric protein, can be administered to a subject using methods known in the art. In particular, the composition can be administered intravenously, subcutaneously, intramuscularly, intradermally, or via any mucosal surface, e.g., orally, sublingually, buccally, nasally, rectally, vaginally or via pulmonary route. In some embodiments, the administration is intravenous. In some embodiments, the administration is subcutaneous. In some embodiments, the administration is self-administration. In some embodiments, a parent administers the chimeric protein to a child. In some embodiments, the chimeric protein is administered to a subject by a healthcare practitioner such as a medical doctor, a medic, or a nurse.

### II. Messenger RNA (mRNA)

Disclosed herein are mRNA compositions comprising RNA aptamers. mRNA is typically thought of as the type of RNA that carries information from DNA to the ribosome. The existence of mRNA is typically very brief and includes processing and translation, followed by degradation. Typically, in eukaryotic organisms, mRNA processing comprises the addition of a “cap” on the N-terminal (5′) end, and a “tail” on the C-terminal (3′) end.

A typical cap is a 7-methylguanosine cap, which is a guanosine that is linked through a 5′-5′-triphosphate bond to the first transcribed nucleotide. The presence of the cap is important in providing resistance to nucleases found in most eukaryotic cells. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase.

The tail is typically a polyadenylation event whereby a polyadenylyl moiety is added to the 3′ end of the mRNA molecule. The presence of this “tail” serves to protect the mRNA from exonuclease degradation. Messenger RNA is translated by the ribosomes into a series of amino acids that make up a protein.

In some embodiments, mRNAs include a 5′ and/or 3′ untranslated region (UTR). In some embodiments, mRNA disclosed herein comprise a 5′ UTR that includes one or more elements that affect an mRNA's stability or translation. In some embodiments, a 5′ UTR may be between about 50 and 500 nucleotides in length. In some embodiments, mRNA disclosed herein comprise a 3′ UTR comprising one or more of a polyadenylation signal, a binding site for proteins that affect an mRNA's stability of location in a cell, or one or more binding sites for miRNAs. In some embodiments, a 3′ UTR may be between 50 and 500 nucleotides in length or longer. In some embodiments, the mRNAs disclosed herein comprise a 5′ or 3′ UTR that is derived from a gene distinct from the one encoded by the mRNA transcript. In some embodiments, the mRNAs disclosed herein comprise a 5′ or 3′ UTR that is chimeric.

The mRNAs disclosed herein may be synthesized according to any of a variety of known methods. For example, mRNAs according to the present invention may be synthesized via in vitro transcription (IVT). Methods for in vitro transcription are known in the art. See, e.g., Geall et al. (2013) Semin. Immunol. 25 (2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application. The presence of these reagents is undesirable in a final mRNA product and are considered impurities or contaminants which must be purified to provide a clean and homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

The methods disclosed herein may be used to purify mRNA of a variety of nucleotide lengths. In some embodiments, the disclosed methods may be used to purify mRNA of greater than about 1 kb, 1.5 kb, 2 kb, 2.5 kb, 3 kb, 3.5 kb, 4 kb, 4.5 kb, 5 kb, 6 kb, 7 kb, 8 kb, 9 kb, 10 kb, 11 kb, 12 kb, 13 kb, 14 kb, or 15 kb in length. The mRNA disclosed herein may be modified or unmodified. In some embodiments, the mRNA disclosed herein contain one or more modifications that typically enhance RNA stability. Exemplary modifications include include backbone modifications, sugar modifications, or base modifications. In some embodiments, the disclosed mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, ß-D-mannosyl-queosine, phosphoramidates, phosphorothioates, peptide nucleotides, methylphosphonates, 7-deazaguanosine, 5-methylcytosine, and inosine. In some embodiments, the disclosed mRNAs comprise at least one chemical modification including but not limited to, consisting of pseudouridine, N1-methylpseudouridine, 2-thiouridine, 4′-thiouridine, 5-methylcytosine, 2-thio-I-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-I-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some embodiments, the modified nucleotides comprise N1-methylpseudouridine. The preparation of such analogues is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

In some embodiments, the mRNAs disclosed herein contains mRNA derived from a single gene or a single synthesis or expression construct. However, in some embodiments, the mRNA compositions disclosed herein comprise multiple mRNA transcripts and each can or collectively code for one or more proteins.

In some embodiments, the mRNA comprising the RNA aptamer as disclosed herein encodes a therapeutic polypeptide. In some embodiments, the therapeutic polypeptide comprises an antibody heavy chain, an antibody light chain, an enzyme, or a cytokine.

In some embodiments, the mRNA encodes a cytokine. Non-limiting examples of cytokines include IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-α, INF-γ, GM-CFS, M-CSF, LT-β, TNF-α, growth factors, and hGH.

In one embodiment, the mRNA comprising the RNA aptamer encodes a genome-editing polypeptide. In some embodiments, the genome-editing polypeptide is a CRISPR protein, a restriction nuclease, a meganuclease, a transcription activator-like effector protein (TALE, including a TALE nuclease, TALEN), or a zinc finger protein (ZF, including a ZF nuclease, ZFN). See, e.g., Int'l Pub. No. WO2020139783.

In some embodiments, the mRNA encodes an enzyme that is utilized in an enzyme replacement therapy. Examples of enzyme replacement therapy include lysosomal diseases, such as Gaucher disease, Fabry disease, MPS I, MPS II (Hunter syndrome), MPS VI and Glycogen storage disease type II.

In some embodiments, the mRNA comprising the RNA aptamer encodes an antigen of interest. The antigen may be a polypeptide derived from a virus, for example, influenza virus, coronavirus (e.g., SARS-COV-1, SARS-COV-2, or MERS-related virus), Ebola virus, Dengue virus, human immunodeficiency virus (HIV), hepatitis A virus (HAV), hepatitis B virus (HBV), hepatitis C virus (HCV), herpes simplex virus (HSV), respiratory syncytial virus (RSV), rhinovirus, cytomegalovirus (CMV), zika virus, human papillomavirus (HPV), human metapneumovirus (hMPV), human parainfluenza virus type 3 (PIV3), Epstein-Barr virus (EBV), or chikungunya virus.

The antigen may be derived from a bacterium, for example, Staphylococcus aureus, Moraxella (e.g., Moraxella catarrhalis; causing otitis, respiratory infections, and/or sinusitis), Chlamydia trachomatis (causing chlamydia), borrelia (e.g., Borrelia burgdorferi causing Lyme Disease), Bacillus anthracis (causing anthrax), Salmonella typhi (causing typhoid fever), Mycobacterium tuberculosis (causing tuberculosis), Propionibacterium acnes (causing acne), or non-typeable Haemophilus influenzae.

Where desired, the mRNA comprising the RNA aptamer may encode for more than one antigen. In some embodiments, the mRNAs disclosed herein encode for two, three, four, five, six, seven, eight, nine, ten, or more antigens. These antigens can be from the same or different pathogens. For example, a polycistronic mRNA that can be translated into more than one antigen (e.g., each antigen-coding sequence is separated by a nucleotide linker encoding a self-cleaving peptide such as a 2A peptide) and can be further fused to the aptamer.

In some embodiments, the mRNA compositions disclosed herein are used in a vaccine. mRNA vaccines provide a promising alternative to traditional subunit vaccines, which contain antigenic proteins derived from a pathogen. Vaccines based on mRNA allow de novo expression of complex antigens in the vaccinated subject, which in turn allows proper post-translational modification and presentation of the antigens in its natural conformation. Moreover, once established, the manufacturing process for mRNA vaccines can be used for a variety of antigens, enabling rapid development and deployment of mRNA vaccines. A detailed discussion of mRNA vaccines can be found in Pardi, et al. (2018) Nat Rev Drug Discov 17, 261-279.

### III. Aptamers

Widespread use of affinity purification of RNA has been limited due to the lack of efficient RNA fusion tags. Unless the RNA to be purified naturally contains a sequence with strong affinity for a target that can be immobilized on the stationary phase (i.e., a chromatography resin), the RNA may require tagging with a specific sequence to do so, analogous to the polyhistidine tag used in protein science.

Disclosed herein are mRNA compositions which comprise at least one aptamer. The aptamers associated with these mRNA compositions enable the use of affinity purification with minimal impact on translation efficiency and immunogenicity. Also disclosed herein are methods of making such mRNA-tagged aptamer compositions.

The term “aptamer” as used herein refers to any nucleic acid sequence that has a non-covalent binding site for a specific target. Exemplary aptamer targets include nucleic acid sequence, protein, peptide, antibody, small molecule, mineral, antibiotic, and others. The aptamer binding site may result from secondary, tertiary, or quaternary conformational structure of the aptamer.

The term “RNA aptamer” as used herein refers to an aptamer comprised of RNA. In some embodiments, the RNA aptamer is included in the nucleotide sequence of the mRNA transcript. In other embodiments, the RNA aptamer is separate from the nucleotide sequence of the mRNA transcript.

Aptamers are typically capable of binding to specific targets with high affinity and specificity. Aptamers have several advantages over other binding proteins (e.g. antibodies). For example, aptamers can be engineered completely in vitro (e.g., via a SELEX aptamer selection method), can be produced by chemical synthesis, possess desirable storage properties, and elicit little or no immunogenicity in therapeutic applications. See, generally, Proske et al., (2005) Appl. Microbiol. Biotechnol 69:367-374.

Aptamers have historically been used to modulate gene expression by directly binding to ligands. These aptamers act similarly to regulatory proteins, forming highly specific binding pockets for the target, followed by conformational changes.

In some embodiments, the RNA aptamer is synthetically derived. In some embodiments, the RNA aptamer is naturally derived from prokaryotes and/or eukaryotes. In some embodiments, the RNA aptamer is derived from a hairpin RNA, a tRNA, or a riboswitch.

In some embodiments the RNA aptamer is derived from a riboswitch. Riboswitches are regulatory RNA elements that act as small molecule sensors to control gene transcription and translation. Several riboswitch classes are known in the art. Exemplary riboswitches include B₁₂ riboswitch, TPP riboswitch, SAM riboswitch, guanine riboswitch, FMN riboswitch, lysine riboswitch, and the PreQ1 riboswitch.

In some embodiments, the RNA aptamer is a split aptamer. Split aptamers are analogs to split-protein systems (e.g. beta-galactosidase) and rely on two or more short nucleic acid strands that assemble into a higher order structure upon the presence of a specific target. Debais et al. (2020) Nucleic Acids Res 48 (7): 3400-3422. An exemplary split aptamer is the ATP-aptamer. Sassanfar & Szostak (1993) Nature 364 (6437)-550-553. The ATP aptamer is an RNA aptamer that was divided into two RNA fragments by removing the loop that closes the stem and by extending each fragment with additional nucleotides to compensate for the loss of stability. Neither of the two RNA fragments bind ATP alone but in the presence of ATP the binding ability is reactivated. Debiais et al. (2020) Nucleic Acids Res 48 (7): 3400-3422.

In some embodiments, the RNA aptamer is an X-aptamer. X-aptamers are engineered with a combination of natural and chemically-modified nucleotides to improve binding affinity, specificity, and versatility. An exemplary embodiment of a X-aptamer is the PS2-aptamer. The PS2-aptamer is an RNA aptamer that contains a phosphorodithioate (i.e., PS2) substitution at a single nucleotide of RNA aptamer which increases the aptamer's binding affinity from a nanomolar to a picomolar range. Abeydeera et al. (2016) Nucleic Acids Res. 44 (17): 8052-8064.

In some embodiments, the RNA aptamer binds to a ligand. In some embodiments the ligand is utilized in an affinity purification system. In some embodiments, the affinity ligand comprises protein A, protein G, streptavidin, glutathione (GSH), dextran (sephadex), cellulose (e.g., diethylaminoethyl cellulose) or a fluorescent molecule. In some embodiments, the affinity ligand is immobilized on a chromatography resin.

In some embodiments, the affinity ligand comprises protein A. DNA aptamers have been shown previously to target protein A. See, e.g., Stoltenburg et al. (2016) Sci Rep. 6:33812.

In some embodiments, the disclosed RNA aptamers bind streptavidin. Streptavidin-binding aptamers are described in, e.g., Srisawat & Engelke (2001) RNA 7 (4): 632-641.

Also disclosed herein are RNA aptamers that bind to sephadex. Sephadex-binding aptamers are described in, e.g., Srisawat et al. (2001) Nucleic Acid Res 29 (2): e4.

Also disclosed herein are RNA aptamers that bind to glutathione (GSH). Glutathione-binding aptamers are described in, e.g., Bala, et al. (2011). RNA Biology 8 (1): 101-111. In some embodiments, the RNA aptamer is GSHapt 8.17 or GSHapt 5.39.

Also disclosed herein are RNA aptamers that bind to a fluorescent molecule. Examples of such aptamers are described in, e.g., Paige et al. (2011) Science 333 (6042): 642-646.

In some embodiments, the RNA aptamer comprises a Sim aptamer. In some embodiments, the S1m aptamer used according to the instant disclosure is the aptamer described in Bachler et al. (1999) RNA 5 (11): 1509-1516, Srisawat & Engelke (2001) RNA 7 (4): 632-641, or Li & Altman. (2002) Nuc. Acids Res. 30 (17): 3706-3711. In some embodiments, the RNA adapter comprises the nucleotide sequence of SEQ ID NO: 2.

In some embodiments, the RNA aptamer comprises a Sm aptamer. In some embodiments, the RNA adapter comprises the nucleotide sequence of SEQ ID NO: 6.

### A. Aptamer Location

The introduction of aptamers into mRNA has been reported to impact translation. The location of the aptamer on the mRNA may partially determine the magnitude of impact on translation. For example, it is generally believed that when inserting structured RNA into a 5′-UTR of a transcript, protein translation levels may be reduced. Babendure et al, (2006). RNA 12:851-861; Kotter et al. (2009) Nuc Acids Res 37 (18): e120. Insertion of an aptamer into the 5′ UTR an mRNA molecule can form a hairpin loop, which alters the structure of the mRNA and blocks access to the ribosome, thereby preventing translation. See, e.g., United States Patent Application Publication No. 2007/0136827.

Disclosed herein are RNA aptamers which include aptamers at various locations with respect to the ORF of the mRNA. Selection of location of the RNA aptamer on the mRNA can be evaluated with respect to both the magnitude of regulation of translation and basal expression level. For example, reporter constructs may be built which contain an aptamer at various locations within the 5′-UTR, between 0 to 100 bases from the cap or start codon. In some embodiments, the downstream region after the aptamer can be retained in order to preserve the peptide leader sequence, thereby limiting alteration to the upstream sequence relative to the aptamer.

In some embodiments, the RNA aptamer is positioned in the 5′ UTR. In some embodiments, the RNA aptamer is positioned following the 5′UTR and immediately before the protein-coding ORF. In some embodiments, the RNA aptamer is positioned following the protein-coding open reading frame (ORF) and immediately before the 3′ UTR. In some embodiments, the RNA aptamer is positioned between the 3′ end of the ORF and the 5′ end of the 3′ UTR. In some embodiments, the RNA aptamer is positioned in the 3′UTR. In some embodiments, the RNA aptamer is positioned downstream of the 3′UTR and immediately before the polyA tail. In some embodiments, the RNA aptamer is positioned between the 3′ end of the 3′UTR and the 5′ end of the polyA sequence. In some embodiments, the RNA aptamer is positioned immediately after the polyA tail (i.e., at the end of the transcript). In some embodiments, the RNA aptamer is positioned at the 3′ end of the polyA sequence.

In some embodiments, the RNA aptamer does not have to be bound directly to the mRNA. In some embodiments, the RNA aptamer is attached to a linker. See, e.g., Elenko et al. (2009) J Am Chem Soc. 131 (29): 9866-9867.

In some embodiments, the RNA aptamer can be removed from the mRNA after affinity purification. This may be achieved, for example, using DNA oligonucleotides which hybridize to the RNA aptamer or RNA scaffold. The resulting duplex can then be cleaved with an enzyme such as RNase H. See, e.g., Batey R T. (2014). Curr Opin Struct Biol. 26:1-8.

### B. Aptamer Copy Number

An increase in aptamer copy number may allow aptamers to create a larger three-dimensional structure (i.e., enhancing the number of affinity ligand binding sites available or creating a unique ligand binding site). A strategic arrangement of aptamer copies may allow for increased avidity with the cognate affinity ligand.

In some embodiments, the mRNA used in the disclosed methods and compositions comprises multiple copies of an aptamer. Previous reports have shown that using a single small-molecule binding aptamer in the 5′-UTR enables 8-fold repression of translation upon ligand addition, but using three aptamers causes a 37-fold repression. Kotter et al., (2009). Nucleic Acids Res. 37 (18): e120. In some embodiments, the copy number of aptamers introduced into the mRNA is one, two, three, four, five, six, seven, eight, nine, ten, or more.

In some embodiments, the RNA aptamer comprises multiple copies of an aptamer sequence. In some embodiments, the RNA aptamer comprises the nucleotide sequence of SEQ ID NO: 5.

In some embodiments, copies of the aptamer are in repeat tandem configuration. The 4×S1m aptamer disclosed herein is an example of a multiple copy aptamer in a repeat tandem configuration.

### IV. RNA Scaffolds

In some embodiments, the mRNA compositions disclosed herein comprise an RNA aptamer that is embedded in an RNA scaffold. As used herein, the term “RNA scaffold” refers to a noncoding RNA molecule that can assemble to have a predefined structure which creates spatial architecture to organize, protect, or enhance the properties of a functional module of interest. Exemplary functional modules can be nucleic acids (e.g., aptamers) or protein. In some embodiments, the RNA scaffolds suitable for use according to the instant disclosure can be associated with an RNA without disrupting the RNA structure. Furthermore, suitable RNA scaffolds allow for an RNA aptamer to be embedded without disrupting the RNA structure. In some embodiments, the RNA scaffolds used according to the instant disclosure can be any RNA scaffolds which do not have a significant negative impact on RNA expression or translation.

An RNA scaffold's predefined structure contains RNA-specific sequence motifs for self-assembly such as base-pairing between hairpin stems (kissing loops) and/or chemical modifications, Myhrvold & Silver (2015) Nat Struct Mol Bio 22 (1): 8-10. RNA-specific sequence motifs can form secondary (i.e., two-dimensional) and/or tertiary (i.e., three-dimensional) structures. In some embodiments, the RNA scaffold comprises at least one secondary structure motif. In some embodiments, the RNA scaffold comprises at least one tertiary structure motif. Common secondary and/or tertiary RNA structural motifs include open and stacked three-way junctions, four-way junctions, four-way junctions similar to Holliday's structures, stem-loops (i.e., hairpin loops), interior loops (i.e., internal loops), bulges, tetraloops, multibranch loops, pseudoknots and knots, 90° kinks, and pseudo-torsional angles. Shanna et al. (2021) Molecules 26 (5): 1422.

RNA scaffolds can either be derived from nature (e.g., attenuators, tRNA, riboswitches, terminators) or artificially engineered to form secondary or tertiary RNA structure. Delebecque et al. (2012) Nat Protoc 7 (10): 1797-1807. Typically, in order to retain the RNA scaffold predefined structure, the RNA scaffold's RNA loop(s) (e.g., a hairpin loop) are the target regions for embedding the functional module of interest. See, e.g., U.S. Pat. No. 20050282190 A1. The RNA scaffold's predefined structure can be modified, however, to have additional desirable properties. For example, the predefined RNA scaffold structure may be modified to become resistant to one or both of exonuclease digestion and endonuclease digestion.

In some embodiments, the mRNA compositions disclosed herein comprise an RNA aptamer that is embedded in a transfer RNA (tRNA). Transfer RNA (tRNA) scaffolds are an attractive tagging candidate in affinity purification systems, as tRNAs fold into canonical, stable clover-leaf structures that are resistant to unfolding and can protect RNA fusions from nuclease degradation. It has been demonstrated that embedding an aptamer in the anticodon loop of a tRNA scaffold promotes proper folding. See generally, Ponchon and Dardel (2007) Nat. Methods 4 (7): 571-576; Ponchon et al. (2013) Nucleic Acids Res. 41: e150. Use of an RNA aptamer embedded in a tRNA scaffold has been demonstrated to successfully pull-down transcript-specific RNA-binding proteins from cell lysates. Iioka H et al. (2011) Nuc. Acids Res. 39 (8): e53.

In some embodiments, the mRNA compositions disclosed herein comprise an RNA aptamer that is embedded in a tRNA which comprises the nucleotide sequence of SEQ ID NO: 7.

In some embodiments, the RNA aptamer is embedded in a tRNA hairpin loop of the tRNA. In some embodiments, the RNA aptamer is embedded in a tRNA anticodon loop. In some embodiments, the RNA aptamer is embedded in a tRNA D loop. In some embodiments, the RNA aptamer is embedded in a tRNA T loop.

In some embodiments, the mRNA compositions disclosed herein comprise an RNA aptamer embedded in a bioorthogonal scaffold. The hallmark feature of a bioorthogonal scaffold is that it is not recognized by intracellular nucleases and targeted for degradation. Filonov et al. (2015) Chem Biol. 22 (5): 649-660. Examples of bioorthogonal scaffolds include, V5, F29, F30, or variants thereof. Id. F29 and F30 share the same three-way junction motif that is seen in naturally occurring riboswitches and viral RNAs. Shu et al. (2014) Nucleic Acids Res. 42, e10. F30 is an engineered version of F29 which was mutated to remove an internal terminator sequence. Filonov et al. (2015) Chem Biol. 22 (5): 649-660.

In some embodiments, the mRNA compositions disclosed herein comprise an RNA aptamer embedded in a bioorthogonal scaffold. In some embodiments, the bioorthogonal scaffold is V5, F29, F30, or a variant thereof.

In some embodiments, the bioorthogonal scaffold comprises a 5′ nucleotide sequence of SEQ ID NO: 34 and a 3′ nucleotide sequence of SEQ ID NO: 35, wherein an aptamer sequence is positioned between SEQ ID NO: 34 and SEQ ID NO: 35.

In some embodiments, the bioorthogonal scaffold comprises a 5′ nucleotide sequence of SEQ ID NO: 39, an internal nucleotide sequence of SEQ ID NO: 40, and a 3′ nucleotide sequence of SEQ ID NO: 41, wherein a first aptamer sequence is positioned between SEQ ID NO: 39 and SEQ ID NO: 40 and a second aptamer sequence is positioned between SEQ ID NO: 40 and SEQ ID NO: 41, optionally wherein the first and second aptamer are the same or different.

In some embodiments, the RNA aptamer embedded bioorthogonal scaffold comprises the nucleotide sequence of SEQ ID NO: 29 or SEQ ID NO: 31.

Other exemplary RNA scaffolds include ribosomal RNA (rRNA) and ribozymes. In some embodiments, the RNA aptamer is embedded in a ribosomal RNA. In some embodiments, the ribosomal RNA is a 5S rRNA or a derivative thereof. Exemplary 5S rRNA scaffolds and derivatives thereof are described in further detail in Stepanov et al. (Methods Mol Biol. 2323:75-97. 2021), the contents of which are incorporated herein by reference.

In some embodiments, the RNA aptamer is embedded in a ribozyme. In some embodiments, the ribozyme is catalytically inactive.

In some embodiments, the RNA aptamer is embedded in a T-cassette. In some embodiments, the T-cassette RNA scaffold comprises the sequence GAACGAAACUCUGGGAGCUGCGAUUGGCAGAAUUCCGUUAGCAAGGCCGCAGGACUUG CAUGCUUAUCCUGCGGCGCGGGCGCGUUUCCCGGGUUACGCGCCCGCCUUAAGUGUU UCUCGAGUUGGCACUUAAGCUUGCUAACGGAAUUCCCCCAUAUCCAACUUCCAAUUUAA UCUUUCUUUUUUAAUUUUCACUUAUUUGCG (SEQ ID NO: 43, wherein the bold, underlined text correspond to aptamer insertion sites. An aptamer may be inserted at 1, 2, or all 3 aptamer insertion sites. In some embodiments, the T-cassette RNA scaffold is embedded with 1, 2, or 3 aptamers. In some embodiments, the aptamers are the same. In other embodiments, the aptamers are different. In yet other embodiments, 2 of 3 aptamers are different. In yet other embodiments, 2 or 3 aptamers are the same.

In some embodiments, the T-cassette RNA scaffold is encoded by the polynucleotide sequence of

(SEQ ID NO: 44)
GAACGAAACTCTGGGAGCTGCGATTGGCAGAATTCCGTTAGCAAGG
CCGCAGGACTTGCATGCTTATCCTGCGGCGCGGGCGCGTTTCCCG
GGTTACGCGCCCGCCTTAAGTGTTTCTCGAGTTGGCACTTAAGCT
TGCTAACGGAATTCCCCCATATCCAACTTCCAATTTAATCTTTCT
TTTTTAATTTTCACTTATTTGCG.

The T-cassette scaffold is described in further detail in Wurster et al. (Nucleic Acids Research. 37 (18): 6214-6224. 2009), the contents of which are incorporated herein by reference.

### V. Affinity Purification of RNA

In one aspect, disclosed herein are methods for purifying a mRNA sample. In some embodiments, mRNA purified according to the disclosed methods is substantially free of impurities from mRNA synthesis. These impurities include, for example, prematurely aborted RNA sequences, DNA templates, and/or enzyme reagents used in in vitro synthesis.

In some embodiments, the disclosed method for purifying a mRNA comprises the steps of: (a) contacting a sample comprising a mRNA comprising at least one aptamer with an affinity ligand that is immobilized on a chromatography resin, wherein the RNA aptamer comprises binding affinity for the affinity ligand; (b) eluting the mRNA from the chromatography resin; and (c) purifying the mRNA from the sample.

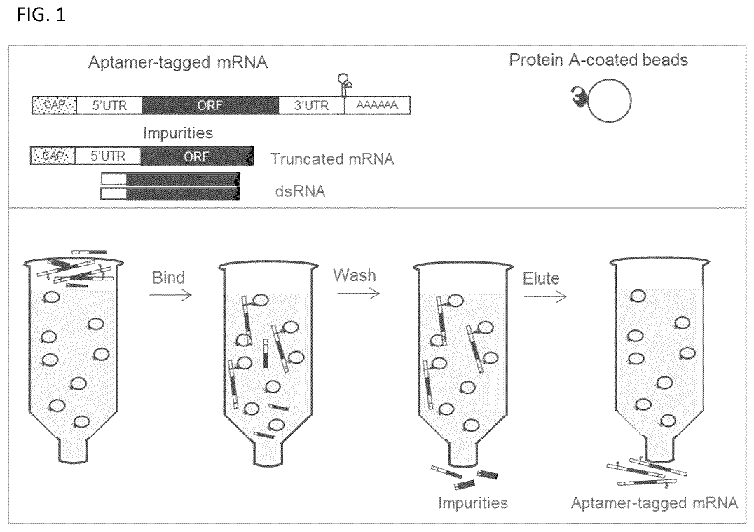

Affinity chromatography is one purification method that can be used with the mRNA compositions and methods disclosed herein. The RNA aptamers disclosed herein comprise binding affinity for the selected affinity ligand. The selected affinity ligand is is immobilized (e.g. crosslinked) on a chromatography resin. The mRNA comprising the RNA aptamer therefore binds with the resin containing the affinity ligand. The chromatography resin material is preferably present in a column, wherein the sample containing RNA is loaded on the top of the column and the eluent is collected at the bottom of the column. See, e.g., FIG. 1 for a general illustration of the affinity purification methods disclosed herein.

The chromatography resin can be any material that is known to be used as a stationary phase in chromatography methods. The type of molecules used as affinity ligands, which interact with the RNA aptamers disclosed herein, can be a variety of types. Non-exhaustive examples of affinity ligands are antibodies, proteins, oligonucleotides, dyes, boronate groups, or chelated metal ions. The stationary phase may be composed of organic and/or inorganic material.

The most widely used stationary phase materials are hydrophilic carbohydrates such as cross-linked agarose and synthetic copolymer materials. These materials may comprise derivatives of cellulose, polystyrene, synthetic poly amino acids, synthetic polyacrylamide gels, or a glass surface. Further examples of materials that can be used as chromatography resins are polystyrenedivinylbenzenes, silica gel, silica gel modified with non-polar residues, or other materials suitable for gel chromatography or other chromatographic methods, such as dextran, sephadex, agarose, dextran/agarose mixtures, and others known in the art.

The chromatography resin can be functionalized with affinity ligands for which the RNA aptamer has binding affinity. In some embodiments, the resin may be an agarose media or a membrane functionalized with phenyl groups (e.g., Phenyl Sepharose™ from GE Healthcare or a Phenyl Membrane from Sartorius), Tosoh Hexyl, CaptoPhenyl, Phenyl Sepharose™ 6 Fast Flow with low or high substitution, Phenyl Sepharose™ High Performance, Octyl Sepharose™ High Performance (GE Healthcare); Fractogel™ EMD Propyl or Fractogel™ EMD Phenyl (E. Merck, Germany); Macro-Prep™ Methyl or Macro-Prep™ t-Butyl columns (Bio-Rad, California); WP HI-Propyl (C3)™ (J. T. Baker, New Jersey) or Toyopearl™ ether, phenyl or butyl (TosoHaas, PA). ToyoScreen PPG, ToyoScreen Phenyl, ToyoScreen Butyl, and ToyoScreen Hexyl are based on rigid methacrylic polymer beads. GE HiScreen Butyl FF and HiScreen Octyl FF are based on high flow agarose based beads. Preferred are Toyopearl Ether-650M, Toyopearl Phenyl-650M, Toyopearl Butyl-650M, Toyopearl Hexyl-650C (TosoHaas, PA), POROS-OH (ThermoFisher) or methacrylate based monolithic columns such as CIM-OH, CIM-SO3, CIM-C4 A and CIM C4 HDL which comprise OH, sulfate or butyl ligands, respectively (BIA Separations).

In some embodiments, the chromatography resin comprises protein A as an affinity ligand. Exemplary protein A resins include Byzen Pro Protein A resin (MilliporeSigma; 18887), Dynabeads Protein A Magnetic Beads (ThermoFisher; 10001D), Pierce Protein A Agarose (ThermoFisher; 20334), Pierce Protein A/G Plus Agarose (ThermoFisher; 20423), Pierce Protein A Plus UltraLink (ThermoFisher; 53142), Pierce Recombinant Protein A Agarose (ThermoFisher), POROS MabCapture A Select (ThermoFisher).

In some embodiments, the chromatography resin comprises streptavidin as an affinity ligand. Exemplary streptavidin resins include Streptavidin-Agarose from Streptomyces avidinii (MilliporeSigma; S1638), Pierce Streptavidin Plus UltaLink Resin (ThermoFisher; 53117), Pierce High Capacity Steptavisin Agarose (ThermoFisher; 20357), Streptavidin 6HC Agarose Resin (ABT; STV6HC-5), Streptavidin Resin-Amintra (Abcam; ab270530).

In some embodiments, the chromatography resin comprises glutathione (GSH) as an affinity ligand. Exemplary GSH resins include Glutathione Resin (GenScript; L00206), Pierce Glutathione Agarose (ThermoFisher; 16102BID), Glutathione Sepharose 4B GST-tagged Protein Resin 9Cytiva; 17075605); Glutathione Affinity Resin-Amintra (Abcam; ab270237).

In certain embodiments, the purification process disclosed herein may be carried out during or subsequent to mRNA synthesis. For example, mRNA may be purified as described herein before a cap and/or tail are added to the mRNA. In some embodiments, the mRNA is purified after a cap and/or tail are added to the mRNA. In some embodiments, the mRNA is purified after a cap is added. In some embodiments, the mRNA is purified both before and after a cap and/or tail are added to the mRNA. In general, a purification step as described herein may be performed after each step of mRNA synthesis, optionally along with other purification processes, such as dialysis and/or filtration. For example, mRNA may undergo dialysis to remove shortmers after initial synthesis (e.g., with or without a tail) and then be subjected to purification as described herein. The purification methods disclosed herein may be applied multiple times to a mRNA sample.

### VI. Vectors

In one aspect, disclosed herein are vectors comprising the mRNA compositions disclosed herein. The nucleic acid sequences encoding a protein of interest (e.g., mRNA encoding a therapeutic polypeptide) can be cloned into a number of types of vectors. For example, the nucleic acids can be cloned into a vector including, but not limited to a plasmid, a phagemid, a phage derivative, an animal virus, and a cosmid. Vectors of particular interest include expression vectors, replication vectors, probe generation vectors, sequencing vectors and vectors optimized for in vitro transcription.

In one embodiment, the vector is used to express mRNA in a host cell. In another embodiment, the vector is used as a template for IVT. The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14, 265-281.

In some embodiments, the vectors disclosed herein comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein also comprise a polynucleotide sequence encoding a polyA sequence and/or a polyadenylation signal.

A variety of RNA polymerase promoters are known in the art. In one embodiment, the promoter is a T7 RNA polymerase promoter. Other useful promoters include, but are not limited to, T3 and SP6 RNA polymerase promoters. Consensus nucleotide sequences for T7, T3 and SP6 promoters are known in the art.

Also disclosed herein are host cells (e.g., mammalian cells, e.g., human cells) comprising the vectors or RNA compositions disclosed herein.

Polynucleotides can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12 (8): 861-70, or the TransIT-RNA transfection Kit (Mirus, Madison WI).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present invention, in order to confirm the presence of the mRNA sequence in the host cell, a variety of assays may be performed. Such assays are well known to those of skill in the art.

### VII. Pharmaceutical Compositions

RNA purified according to this invention is useful as a component in pharmaceutical compositions, for example for use as a vaccine. These compositions will typically include RNA and a pharmaceutically acceptable carrier. A pharmaceutical composition of the invention can also include one or more additional components such as small molecule immunopotentiators (e.g. TLR agonists). A pharmaceutical composition of the invention can also include a delivery system for the RNA, such as a liposome, an oil-in-water emulsion, or a microparticle. In some embodiments, the pharmaceutical composition comprises a lipid nanoparticle (LNP). In one embodiment, the composition comprises an antigen-encoding nucleic acid molecule encapsulated within a LNP. In some embodiments, the LNP comprises at least one cationic lipid. In some embodiments, the LNP comprises a cationic lipid, a polyethylene glycol (PEG) conjugated (PEGylated) lipid, a cholesterol-based lipid, and a helper lipid.

In order that this invention may be better understood, the following examples are set forth. These examples are for purposes of illustration only and are not to be construed as limiting the scope of the invention in any manner.

### EXAMPLES

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### Example 1: Aptamer Synthesis

Two RNA aptamer sequences were chemically synthesized. The first RNA aptamer nucleotide sequence was a random sequence aptamer to serve as a negative control (SEQ ID NO: 1). The second sequence is the S1m aptamer (SEQ ID NO: 2), which was previously reported to bind to streptavidin. Bachler et al., (1999), RNA 5 (11): 1509-1516; Srisawat, C. and Engelke, D. R., (2001) RNA 7 (4): 632-641; Li, Y. and Altman, S., Nucleic Acids Res. (2002), 30 (17): 3706-3711. The nucleotide sequence for the random aptamer (SEQ ID NO: 1) and the Sim aptamer (SEQ ID NO: 2) are shown below.

SEQ ID NO: 1_Random Aptamer Tag (58 bp)
AUACCAGCUUAUUCAAUUAGCAACAUGAG
GGGGAUAGAGGGGGUGGGUUCUCUCGGCU
SEQ ID NO: 2_S1m aptamer Tag (60 bp)
AUGCGGCCGCCGACCAGAAUCAUGCAAGUG
CGUAAGAUAGUCGCGGGUCGGCGGCCGCAU

### Example 2: Streptavidin Sepharose Bead Affinity Purification and RNA Quantification

Binding of the aptamers was analyzed using a sepharose bead affinity purification strategy followed by quantification of the yield of RNA recovery.

Methods for preparing the RNA aptamers and streptavidin beads for binding involved the following steps: (1) Preparation of the streptavidin sepharose beads. To remove bead storage solution, 20 μL of streptavidin sepharose beads (per sample) were spun at 600×g for 1 minute at 4° C. and washed twice in binding buffer (500 μL/per sample). Subsequently, the beads were resuspended in 20 μL of binding buffer with RNasin Ribonuclease Inhibitor (3 μL/100 units) and then incubated on ice for 15 minutes. (2) Preparation of RNA aptamers. 2.5 μg of the RNA aptamers were resuspended in 10 μL binding buffer. Refolding of the RNA aptamers was performed by heating at 56° C. for 5 min, 37° C. for 10 min, followed by a room temperature incubation for 5 minutes to refold aptamer structure. At the end of the RNA aptamer preparation procedure, 2 μL of the random aptamer and the Sim aptamer in a 1:2 mix with binding buffer were collected as a control for total RNA aptamer yield (input control). (3) Incubation conditions. 10 μL of refolded aptamer containing mRNA (2.5 μg) aptamers were added to the beads and incubated at 4° C. for 2 hours on a rotator. Subsequently, beads were washed 3 times with 100 μL of binding buffer and kept on ice for the remainder of the procedure to maintain aptamer secondary structure. (4) Elution of RNA aptamers from beads. Elution was performed with 250 μL phenol-based reagent in the following steps. 50 μL cold chloroform were added to the beads and shaken vigorously for 10 seconds followed by a spin at 12,000×g for 15 minutes (at 4° C.). Each sample's aqueous top phase containing RNA (approximately 125 μL per sample) was added directly to Monarch cleanup columns and manufacturer's instructions were followed (Monarch RNA Cleanup Kit; NEB). RNA was eluted from each Monarch column in 50 μL DEPC-treated water. RNA concentration following streptavidin affinity purification was quantified on a Nanodrop using parameters set by the manufacturer's specifications.

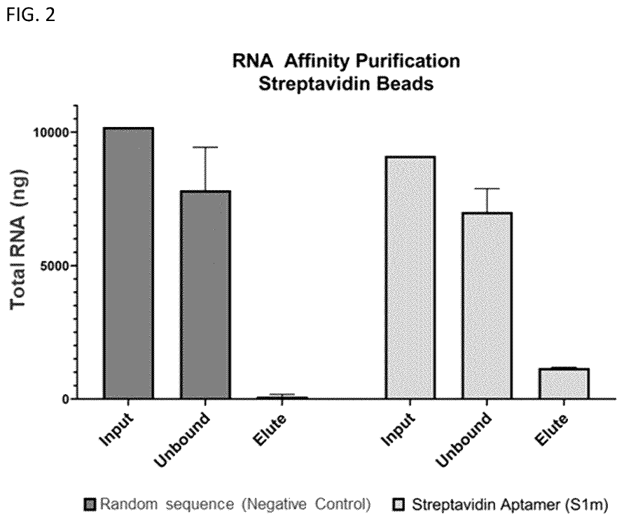

The aptamers prepared in Example 1 were affinity purified with streptavidin sepharose beads, eluted, and the amount of RNA recovery in the eluate was quantified using the methods described above. Random aptamer sequence samples did not yield any RNA recovery (Nanodrop lower detection limit 2.5 ng/μL). In contrast, the S1m aptamer samples had approximately 13% RNA recovery (1,250 ng/μL) relative to S1m aptamer RNA samples collected prior to incubation with streptavidin beads (approximately 9,600 ng/μL) (FIG. 2). This result shows that the S1m aptamers designed in Example 1 can be affinity purified with streptavidin and thus can be suitable as a functional tag in a streptavidin affinity based purification system.

### Example 3: Synthesis and Affinity Purification of mRNA Tagged with Multiple Copy (4×) Aptamer

To analyze the impact of aptamer copy number on binding affinity, a multiple copy aptamer was introduced into mRNA and compared with mRNA which did not include an aptamer.

Arrangement of the S1m aptamer in a tandem four-repeat configuration (4×S1m; SEQ ID NO: 5) was previously shown to have higher affinity to sepharose beads. Leppek & Stoecklin. (2014) Nuc. Acids Res. 42 (2): e13. To study the effect of RNA aptamer copy number on binding affinity, DNA plasmids were constructed to generate the cDNA template for in vitro transcription (IVT) to in order to produce a 4×S1m aptamer tagged to mRNA. Id.

DNA plasmids pAM14 and pAM15 were modified to include a 53 bp nucleotide sequence encoding an AU-rich element (ARE) RNA from the 3′UTR of mouse TNFα driven by a T7 promoter as previously described. Stoeklin G et al., (2004), EMBO J23 (6): 1212-1324; Leppek & Stoecklin. (2014) Nuc. Acids Res. 42 (2): e13. pAM14 (2,496 bp) is derived from the same vector backbone as pAM15 (2,168 bp) but contains a 4×S1m aptamer flanked by a 30-mer polyA tail in a 5′ to 3′ orientation.

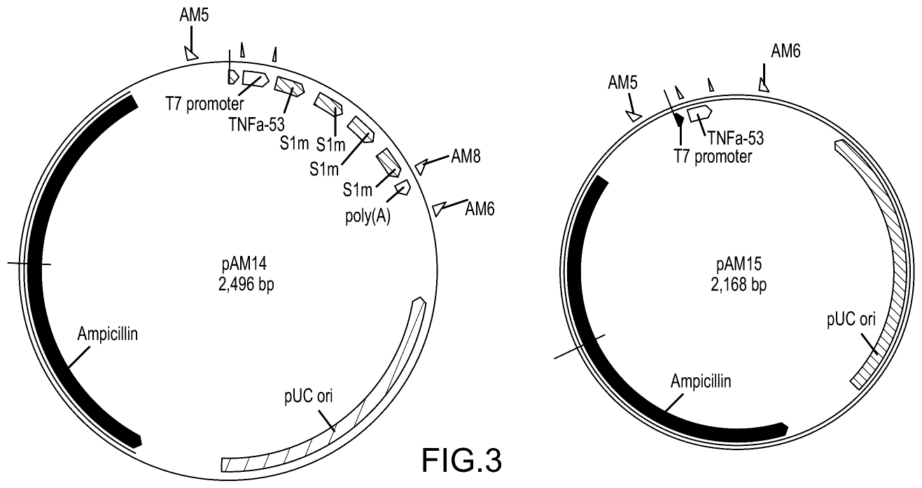

To obtain the cDNA template for IVT (SEQ ID NO: 5) the TNFα-53-4×S1m nucleotide sequence was amplified with an AM5/6 primer pair from the pAM14 plasmid. The negative control cDNA template was amplified using the same AM5/6 primer pair from plasmid pAM15, producing sequences containing 5′ UTR and 3′ UTR flanks (SEQ ID NOs: 3 and 4, respectively). The positions of the AM5/6 primer binding sites are annotated in the pAM14 and pAM15 plasmid maps as shown in FIG. 3.

Subsequently, the IVT reactions for experiment group, TNFα-53-4×S1m mRNA, and control group was carried out using RNA reagents and procedure commercially available. (HiScribe T7 ARCA mRNA synthesis Kit with tailing, NEB). After cap and tail reactions the filtered mRNA was stored at −20° C. until use.

The nucleotide sequences for the 5′UTR, 3′UTR, and the 4×S1m aptamer are shown below.

SEQ ID NO: 3_5′UTR (104 bp)
AGAGCGGCCGCTTTTTCAGCAAGATTAAGCCCAGGGCAG
AGCCATCTATTGCTTACATTTGCTTCTGACACAACT [symbol]
SEQ ID NO: 4_3′UTR (266 bp)
AGCTCGCTTTCTTGCTGTCCAATTTCTATTAAAGGTTCC
TTTGTTCCCTAAGTCCAACTACTAAACTGGGGGATAT [symbol]
GATTCTGCCTAATAAAAAACATTTATTTTCATTGC
SEQ ID NO: 5_4xS1m Tag (321 bp)
ATGCGGCCGCCGACCAGAATCATGCAAGTGCGTAAGATAG
TCGCGGGTCGGCGGCCGCATCTGCTGGGAAGCT [symbol] TC
GGCGGCCGCATCTGCTGGGAAGCTACGATCCGTAGAAAAT
GCGGCCGCCGACCAGAATCATGCAAGTGCGT [symbol] [symbol]
indicates data missing or illegible when filed

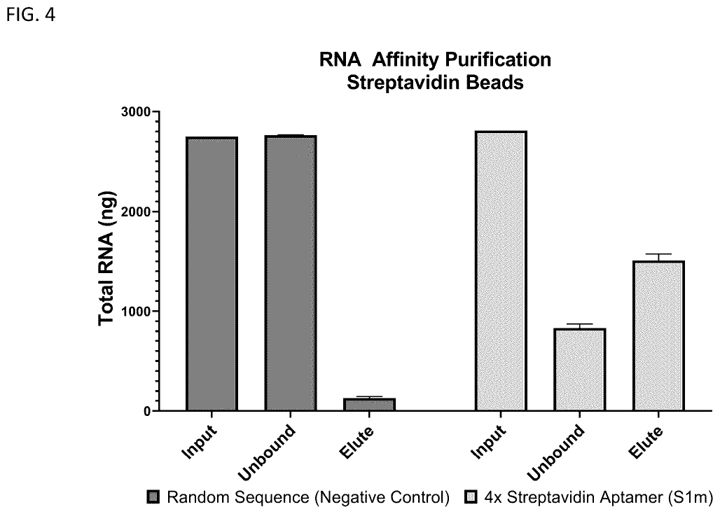

To analyze the affinity binding of TNFα-53-4×S1m aptamer mRNA, the aptamer mRNA was affinity purified with streptavidin sepharose beads, eluted, and the amount of RNA recovery in the eluate was quantified using the methods described above. The binding affinity of streptavidin sepharose beads to a TNFα-53 tagged 4×S1m mRNA or a TNFα-53 mRNA negative control sample was evaluated and compared. Affinity purified TNFα-53 tagged 4×S1m mRNA yielded a 54% RNA recovery yield (1,500 ng/μl) relative to the 4×S1m mRNA samples collected prior to incubation with streptavidin beads (approximately 2,800 ng/μL) (FIG. 4). In contrast, the affinity purified TNFα-53 negative control yielded only a 2% RNA recovery yield. This result shows that introducing multiple aptamer copies (e.g., 4×S1m) can potentially be used to improve the affinity purification efficiency of mRNA.

### Example 4: Synthesis and Affinity Purification of Different mRNAs Tagged with Aptamer Embedded in RNA Scaffold

To test the efficiency of a RNA aptamer embedded in a tRNA scaffold in downstream mRNA affinity purification process four vectors were constructed.

The Sm aptamer was selected for analysis. The nucleotide sequence for the Sm aptamer (SEQ ID NO: 6) and the tRNA-Sm aptamer (SEQ ID NO: 7) are shown below.

SEQ ID NO: 6_Sm Aptamer Tag (37 bp)
CGACCAGAATCATGCAAGTGCGTAAGATAGTCGCGGG
SEQ ID NO: 7_tRNA-Sm Aptamer Tag (111 bp)
GCCCGGAUAGCUCAGUCGGUAGAGCAGCGGCCUCGAC
CAGAAUCAUGCAAGUGCGUAAGAUAGUCGCGGGUCGA
GGCCGCGUCCAGGGUUCAAGUCCCUGUUCGGGCGCCA
SEQ ID NO: 42_DNA encoding_tRNA-
Sm Aptamer Tag (111 bp)
GCCCGGATAGCTCAGTCGGTAGAGCAGCGGCCTCGAC
CAGAATCATGCAAGTGCGTAAGATAGTCGCGGGTCGA
GGCCGCGTCCAGGGTTCAAGTCCCTGTTCGGGCGCCA

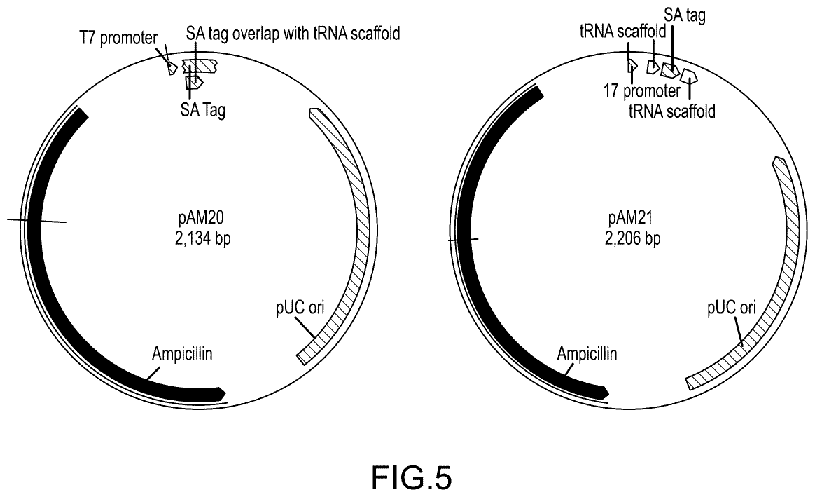

Maps of the plasmids of interest are depicted in FIG. 5. Briefly, these were: (1) pAM22, a control construct, carrying a Methanothermobacter thermautotrophicust tRNA^{GLN2} scaffold (pAM22 (tRNA); plasmid map annotates the position of the anticodon arms with respect to the Gln2 anticodon loop) (2) pAM20, a control construct, carrying a Sm aptamer (pAM20 (Sm)), (3) pAM21, an experimental construct, carrying the Sm aptamer sequence embedded in a portion of the anticodon loop tRNA^{GLN2} sequence which is flanked on both sides by the tRNA anticodon arm sequence (pAM21 (tRNA Sm), and (4) pAM23, an experimental construct, carrying tandem two-repeat configuration of the Sm-tRNA^{GLN2} construct (2× tRNA Sm). Each tag was driven by a T7 promoter.

To obtain the cDNA template for IVT, the aptamer tag nucleotide sequences were amplified with flanking primers, as described in Example 3. [Subsequently, the IVT reactions for experiment group, tRNA Sm and the 2× tRNA Sm mRNA and control group was carried out using RNA reagents and procedure commercially available. (HiScribe T7 ARCA mRNA Kit with tailing, NEB). After cap and tail reactions the filtered mRNA was stored at −20° C. until use.

Affinity binding of the Sm, tRNA, tRNA-Sm, and 2× tRNA Sm aptamer tags were analyzed. The same binding and elution methods from Example 2 were applied.

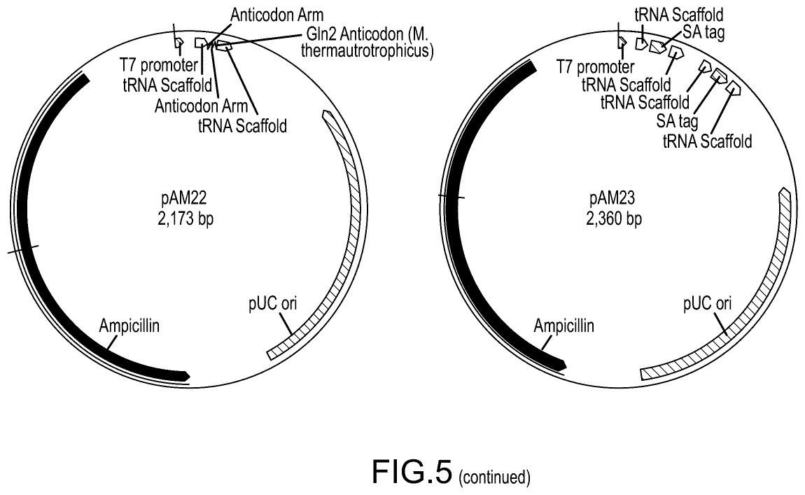

As shown in FIG. 6, use of either the tRNA (pAM22) or the Sm aptamer (pAM20) tags led to similar level of RNA recovery, indicative of non-specific binding under the experimental conditions tested. In contrast, use of the Sm aptamer embedded in a tRNA scaffold in one (pAM21) or two copies (pAM23) was shown to significantly improve purification efficiency, leading to 60% RNA recovery relative to the input RNA. This result demonstrates that use of a RNA scaffold structure, such as a tRNA, can improve the binding efficiency of an aptamer tag.

### Example 5: Synthesis and Affinity Purification of mRNA Encoding hEGFP Tagged with Multiple Copy Aptamer (eHGFP-4×S1m)

This example studies the effect of including RNA aptamer tags on expression of mRNA and protein translation. Since aptamers are designed to be part of the mRNA, there is a possibility that an aptamer tag could negatively impact translation.

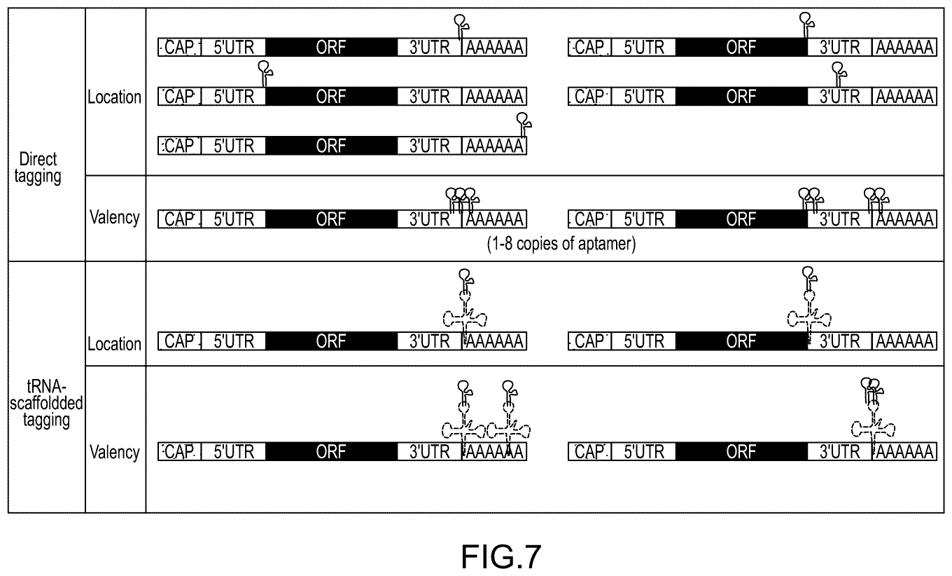

To test the potential impact of RNA aptamers on translation efficiency, plasmids were constructed which included the ORF for humanized enhanced green fluorescent protein (hEGFP; SEQ ID NO: 8 as shown below) flanked by 5′ and 3′ UTR sequences, driven by a T7 promoter, and ending in a 30-mer polyA tail in a 5′ to 3′ orientation (pAM11). Experimental plasmid pAM8 was created by introducing the 4×S1m aptamer sequence (SEQ ID NO: 5) downstream of the 3′ UTR and immediately before the polyA tail. FIG. 8 depicts the plasmid maps of pAM11 and pAM8.

SEQ ID NO: 8_hEGFP (720 bp)
ATGGTGAGCAAGGGCGAGGAGCTGTTCACCGGGGTGGTGCCCATC
CTGGTCGAGCTGGACGGCGACGTAAAC [symbol] GCTACCCCGACCACATG
AAGCAGCACGACTTCTTCAAGTCCGCCATGCCCGAAGGCTACGTC
CAGGAGCGCAC [symbol] GCCACAACGTCTATATCATGGCCGACAAGCAGA
AGAACGGCATCAAGGTGAACTTCAAGATCCGCCACAACATC [symbol] TGG
AGTTCGTGACCGCCGCCGGGATCACTCTCGGCATGGACGAGCTGT
ACAAGTAA [symbol]
indicates data missing or illegible when filed

To obtain the IVT cDNA template, the hEGFP or the hEGFP-4×S1m aptamer tagged nucleotide sequence was amplified with an AM5/6 primer pair. Design and orientation of the primer pair is similar to the strategy as disclosed in Example 3. The IVT reaction was performed with HiScribe™ T7 ARCA mRNA Kit according to manufacturer's instructions. To avoid an additional polyadenylation step, a stretch of 30-mer adenosine tail was created with the template DNA for IVT.

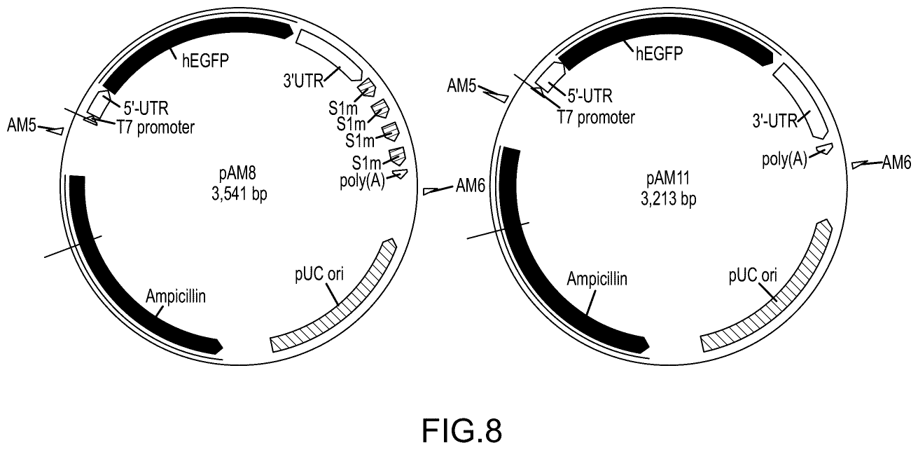

As shown in the agarose gel of FIG. 9 the resulting mRNA are of good quality with expected size (lane 1 hEGFP and lane 2 hEGFP-4×S1m).

To test the effect of the 4×S1m aptamer on affinity binding, the mRNAs containing hEGFP or hEGFP-4×S1m were each affinity purified with streptavidin sepharose beads. The same binding and elution methods as outlined in Example 2 were applied.

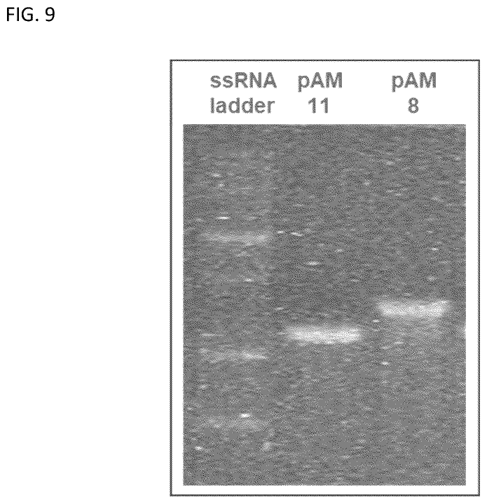

The 4×S1m aptamer tagged hEGFP resulted in a 63% RNA recovery relative to the input control sample, which was significantly higher than the RNA recovery of the hEGFP without aptamer (FIG. 10).

### Example 6: Analysis of Protein Translation and Function of mRNA Tagged with Multiple Copy Aptamer (eHGFP-4×S1m)

The effect of RNA aptamer tags on protein translation and function was assessed by direct visualization of GFP expression in cells. To test this effect, hEGFP mRNA produced from pAM8 and pAM11 was isolated after affinity purification and transfected into HEK293FT cells. 0.5 μg RNA was transfected with Mirus TransIT Transfection reagent into HEK293FT cells in 24-well plates according to manufacturer's instructions. After 24 hours, the cells were examined using fluorescent microscopy.

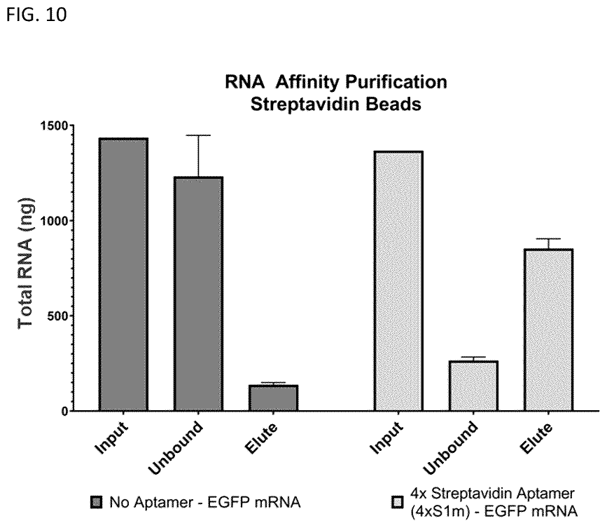

As shown in FIG. 11, the mRNA containing the 4×S1m aptamer produces a lower intensity signal (right panel) relative to mRNA without aptamer (left panel). Thus, it appears that introduction of 4 copies of the streptavidin aptamer tag (4×S1m) may lead to a decrease in translation efficiency of hEGFP expression. This result demonstrates that introducing certain aptamers into mRNA may have an negative impact on protein translation.

### Example 7: Analysis of Protein Translation and Function of mRNA Tagged with Multiple Copy Aptamer and Including Elongated polyA Tail

It was hypothesized that the short polyA tail (30-mer adenosine) may be impacting translation efficiency due to the aptamer sequence. To study the impact of the polyA tail on translation efficiency, hEGFP-4×S1m aptamer tagged mRNA was subjected to an additional polyadenylation reaction using Poly (A) polymerase (NEB, M0276S).

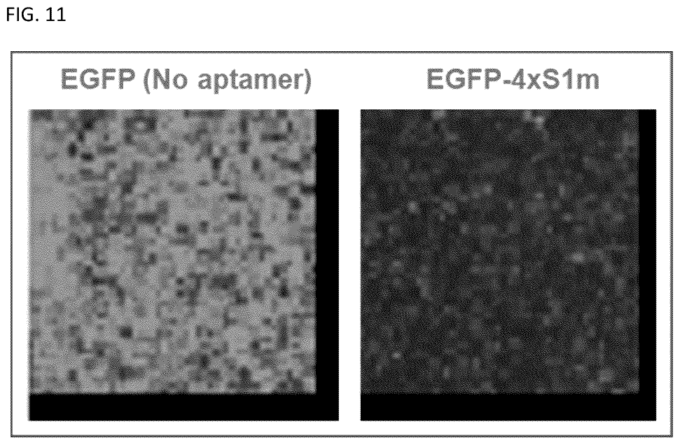

The polyadenylation was confirmed by the shift of the mRNA product on agarose gel (data not shown). mRNA was affinity purified as described above, and mRNA with longer polyA was transfected into HEK293 cells. As shown in FIG. 12, the hEGFP-4×S1m aptamer tagged mRNA with the longer polyA tail showed significantly higher EGFP expression than the mRNA with the shorter (30-mer) polyA tail. This result suggests that the length of the polyA tail may impact the translation efficiency of mRNA which contain certain aptamer sequences.

### Example 8: Analysis of Aptamer Position on RNA Recovery

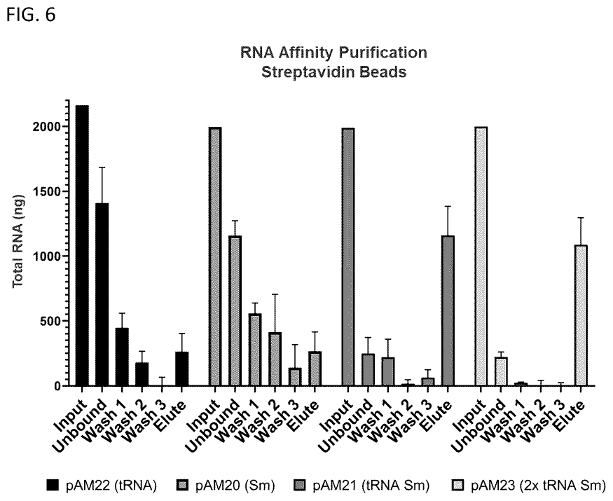

Aptamer sequences are designed to be part of mRNAs, and there is a possibility that the potential aptamer structures or configuration of the same could negatively affect expression. To understand such an impact, aptamer tagged mRNA constructs were designed to test: (1) aptamer position relative to the other topologically ordered mRNA components, (2) aptamer copy number (i.e., aptamer valency), (3) surrounding scaffolding (i.e., a stabilizing tRNA-scaffold), or a combination of configurations as diagrammed in FIG. 7.

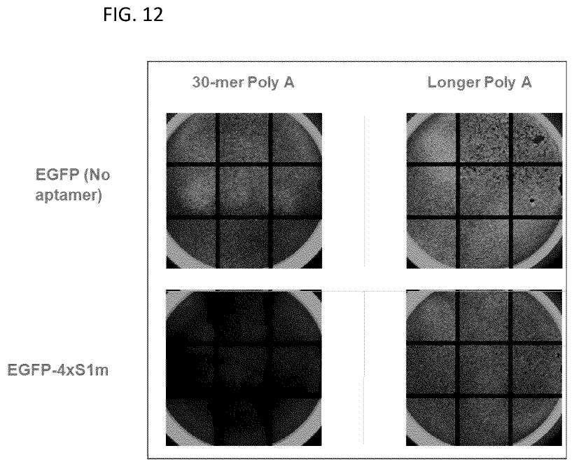

Specifically, this example interrogates whether varying the location of the 4×S1m aptamer sequence with respect to the other topologically ordered pieces in the mRNA impact RNA recovery after mRNA affinity purification. The panel of mRNA constructs designed are shown in FIG. 13A.

Among others, the 4×S1m aptamer was localized either (1) directly upstream of the 5′ UTR, (2) directly upstream of the 3′UTR, (3) in the 3′ UTR, (4) directly downstream the 3′ UTR, or (5) embedded in the 3′ end of the polyA sequence.

cDNA templates were generated and IVT used to produce mRNA with the specific aptamer configuration. mRNA was affinity purified using streptavidin sepharose beads and quantified as described in Example 2.

The affinity purification RNA yield (expressed relative to the input sample that did not undergo affinity purification following streptavidin binding and elution steps) (unbound versus eluted) for each aptamer tagged mRNA tested are shown in FIG. 13B and the average and standard deviation values for each sample (unbound and elute) are shown below in Table 1.

TABLE 1
Percent unbound mRNA and percent eluted
mRNA for the data of FIG. 13B

| | Unbound | | Elute | |
|---|---|---|---|---|
| Plasmid used to | | Standard | | Standard |
| generate mRNA | Average | Deviation | Average | Deviation |
| pAM17 (No aptamer) | 88.5% | 8.7% | 7.3% | 14.5% |
| pAM25 (4xS1m before | 13.0% | 3.2% | 54.0% | 25.1% |
| 5′UTR) | | | | |
| pAM26 (4xS1m before | 15.3% | 1.2% | 46.2% | 22.3% |
| 3′UTR) | | | | |
| pAM27 (4xS1m bisecting | 17.0% | 1.9% | 51.5% | 20.0% |
| 3′UTR) | | | | |
| pAM28 (4xS1m after | 19.7% | 0.5% | 41.8% | 13.4% |
| 3′UTR) | | | | |
| pAM29 (4xS1m after | 16.3% | 2.3% | 50.8% | 6.4% |
| polyA 30-mer) | | | | |

As shown in FIG. 13B and Table 1, the mRNA containing the 4×S1m aptamer, regardless of aptamer location, produced specific binding relative to the control mRNA lacking an aptamer. This result demonstrates that introducing a 4×S1m aptamer into one of multiple locations in the mRNA does not have an impact on the affinity purification yield.

### Example 9: Analysis of Aptamer Valency on RNA Recovery

Like the aptamer position within the mRNA transcript, aptamer valency (i.e., aptamer copy number) is another variable that could impact RNA recovery. To expand on the analysis performed in Example 3, a panel of aptamer tagged mRNA constructs were designed to contain between one to six tandem repeat copies (labeled as 1×S1m through 6×S1m) of the S1m aptamer. For this study, the aptamer tag was placed after the 3′ UTR.

cDNA templates were generated and IVT used to produce mRNAs with specific aptamer valency. mRNA was affinity purified using streptavidin sepharose beads and quantified as described in Example 2.

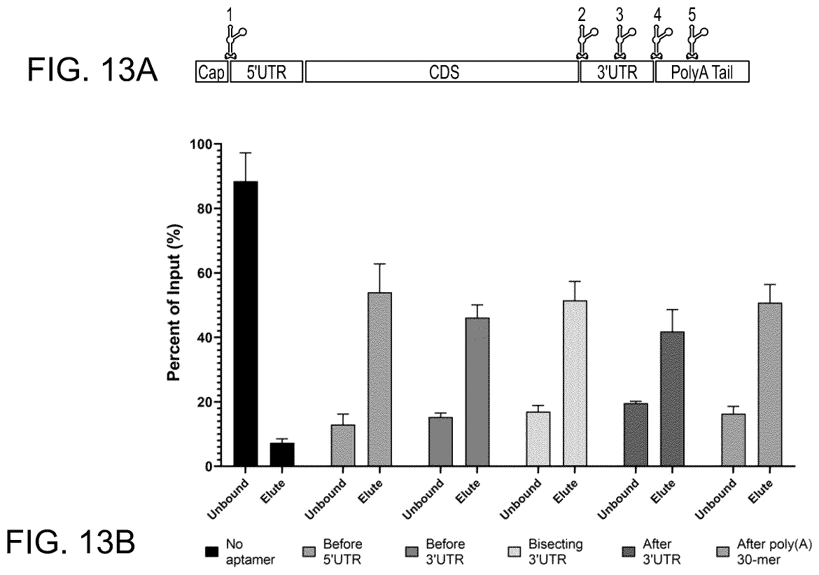

The affinity purification RNA yield (unbound versus eluted) for each aptamer valency mRNA construct tested is shown in FIG. 14 and the average and standard deviation values for each sample (unbound and elute) are shown in Table 2 below.

TABLE 2
Percent unbound mRNA and percent eluted
mRNA for the data of FIG. 14

| | Unbound | | Elution | |
|---|---|---|---|---|
| Plasmid used to | | Standard | | Standard |
| generate mRNA | Average | Deviation | Average | Deviation |
| pAM17 | 72.2% | 2.0% | 11.2% | 0.4% |
| (No aptamer) | | | | |
| pAM30 | 30.7% | 2.2% | 38.1% | 4.5% |
| (1xS1m) | | | | |
| pAM31 | 18.1% | 2.8% | 47.3% | 5.9% |
| (2xS1m) | | | | |
| pAM32 | 20.6% | 0.9% | 52.5% | 2.4% |
| (3xS1m) | | | | |
| pAM28 | 18.5% | 1.6% | 50.4% | 3.5% |
| (4xS1m) | | | | |
| pAM33 | 17.5% | 2.5% | 51.7% | 2.5% |
| (5xS1m) | | | | |
| pAM34 | 16.0% | 0.8% | 57.9% | 7.0% |
| (6xS1m) | | | | |

As shown in FIG. 14, purification efficiency increased for up to three copies of the aptamer (3×S1m) after which no improvement to the RNA affinity purification yield was seen with the addition of subsequent copies (4×S1m-6×S1m). This result demonstrates that increased aptamer valency improves binding affinity.

### Example 10: Analysis of Aptamer Binding in Alternative mRNA Context on RNA Recovery

To demonstrate that the aptamers which provide efficient binding in an affinity purification are functional in alternative RNA contexts, a panel of mRNAs were designed to encode a different protein coding region (Singapore '16 hemagglutinin) and distinct UTR's from what is presented in Example 3.

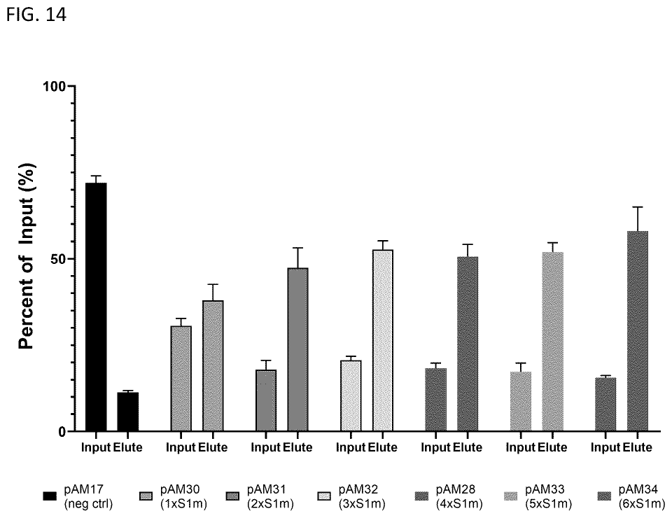

The RNA yield following the streptavidin affinity binding purification process for each construct tested (unbound versus eluted) is shown in FIG. 15. The average and standard deviation values for each sample (unbound and elute) are shown below in Table 3.

TABLE 3
Percent unbound mRNA and percent eluted mRNA for the data of FIG. 15

| | Unbound | | Elution | |
|---|---|---|---|---|
| Plasmid used to | Average Percent | Standard | Average Percent | Standard |
| generate mRNA | Purified | Deviation | Purified | Deviation |
| pAM111 (no aptamer) | 70.7% | 0.4% | 9.8% | 0.9% |
| pAM112 (2xS1m) | 13.4% | 0.2% | 39.0% | 5.4% |
| pAM113 (4xS1m) | 10.3% | 1.2% | 51.0% | 2.5% |
| pAM114 (TRNA S1m) | 9.5% | 0.6% | 50.9% | 2.3% |

As shown in FIG. 15, the aptamers provide specific binding to streptavidin sepharose beads despite the varied neighboring sequence. This result demonstrates that the streptavidin aptamer mRNA designs disclosed herein are robust in alternative RNA contexts.

### Example 11: Analysis of Aptamer Position on Protein Translation

To understand whether mRNA translation kinetics are impacted by aptamer placement within the mRNA transcript, mRNA from the panel of constructs designed in Example 8 were assessed in a mRNA translation efficiency assay to detect GFP expression.

Briefly, mRNA encoding a humanized EGFP (hEGFP) was produced through in vitro transcription (IVT) and subsequently mixed with a transfection reagent. The mix was then applied to either Hela or human skeletal muscle (HSKMc) cells. After 24 hours of incubation, transfected cells were quantified for GFP fluorescence via flow cytometric analysis. The cellular GFP fluorescence intensity being directly proportional to translational efficiency of the mRNA transcript encoding hEGFP.

The following steps describe the transfection procedure for the mRNA translation efficiency assay:
(1) Preparation of cell lines. Hela or HSKMc cell lines were seeded in complete growth media in 12-well plates and grown to an 80-90% confluency. Hela 229 cell media conditions were DMEM and 10% FBS and HSK Mc cells media conditions were 199 Media, 20% FBS, and 1% PenStrep.
(2a) Preparation of mRNA with Mirus TransIT transfection reagent for the HskMc cell line. TransIT-mRNA transfection reagent and mRNA Boost reagents were warmed to room temperature and vortexed gently before using. Following the manufacturer's protocol for mRNA transfection, tubes for each mRNA being tested contained 5 μg of mRNA (10 μL of 500 ng/μL mRNA) to 400 μL of Opti-MEM. For the negative control, media was added instead of mRNA. Subsequently, 8 μL of mRNA Boost Reagent was added and the tube was mixed well by pipetting. Next, 8 μL of Transit mRNA Reagent was added, mixed well, and incubated at room temperature for 2-5 minutes to allow sufficient time for complexes to form.
(3a) Transfection of HskMc cell line. 106.5 μL of the mRNA mix was added dropwise to each well of a 12-well plate (approximately 1.25 μg mRNA/well; triplicate wells were set-up for each construct) and gently rocked to evenly distribute the TransIT-mRNA Reagent: mRNA Boost: RNA complexes. Subsequently, plates were incubated for 24 hours.
(2b) Preparation of mRNA with Lipofectamine MessengerMax transfection reagent for Hela cell line. Following the manufacturer's protocol for mRNA transfection tubes, each mRNA tested was prepared by adding 4 μg mRNA (8 μL of 500 ng/μL mRNA) to 312 μL of Opti-MEM and mixed well. For the negative control, media was added instead of mRNA. In a separate tube, 8 μL of MessengerMax transfection reagent was added to 312 μL of Opti-MEM and mixed well. Subsequently, the volume of the mRNA tube was added to the transfection mixture tube and incubated at room temperature for 15 minutes to allow sufficient time for complexes to form. (3b) Transfection of the Hela cell line. 160 μL/well mRNA mix as described in 2b was added dropwise to each well in a 12-well plate (approximately 1 μg mRNA/well; triplicate wells were set-up for each construct). Subsequently, plates were incubated for 24 hours.

The following steps describe the cell staining and sorting procedure for flow cytometric analysis used in the mRNA translation efficiency assay:
(1) Harvesting cells. Media was aspirated from the cell monolayers, washed with 1 ml of PBS, and dissociated with 250 μL of 1× Accutase per well and incubation at room temperature for 5 min. 250 μL PBS was added to each well and cells were harvested into 1.5 ml microcentrifuge tubes.
(2) Cell Staining. Live/Dead staining was performed on all samples according to manufacturer's instructions (Live/Dead Fixable Far Red Dead Cell Stain Kit). Cell fixation was an optional but not required step. Subsequently, excess stain was removed by washing the cells in 1 ml of PBS, a 300 rcf for 5 minutes at 20° C. was conducted to pellet cells, old supernatant was removed, and samples were resuspended in 400 μL of Stain Buffer (BD Biosciences).
(3) Compensation beads. Compensation control samples were made by preparing live/dead reactive ArC compensation beads or using GFP BrightComp eBeads according to manufacturer's instructions. (4) FACS analysis. For the HskMc and Hela cell lines the 130 μm sorting chip was used. Unstained beads and stained compensation beads were initially run to adjust the FSC/SSC voltage settings and set-up gating windows.

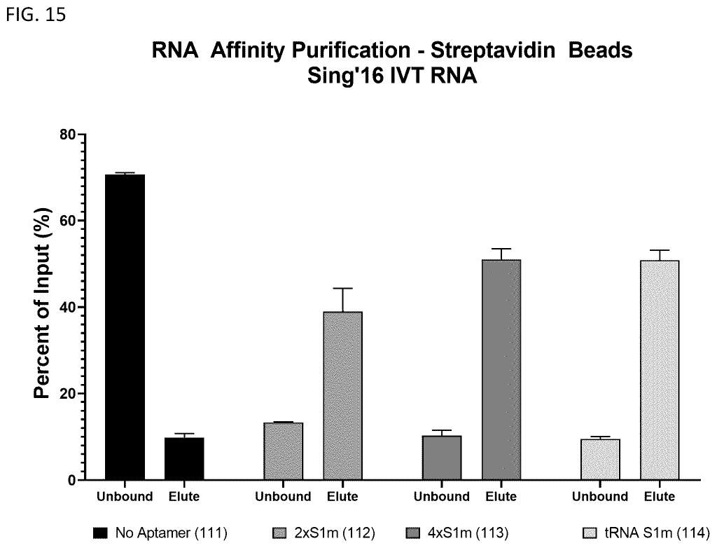

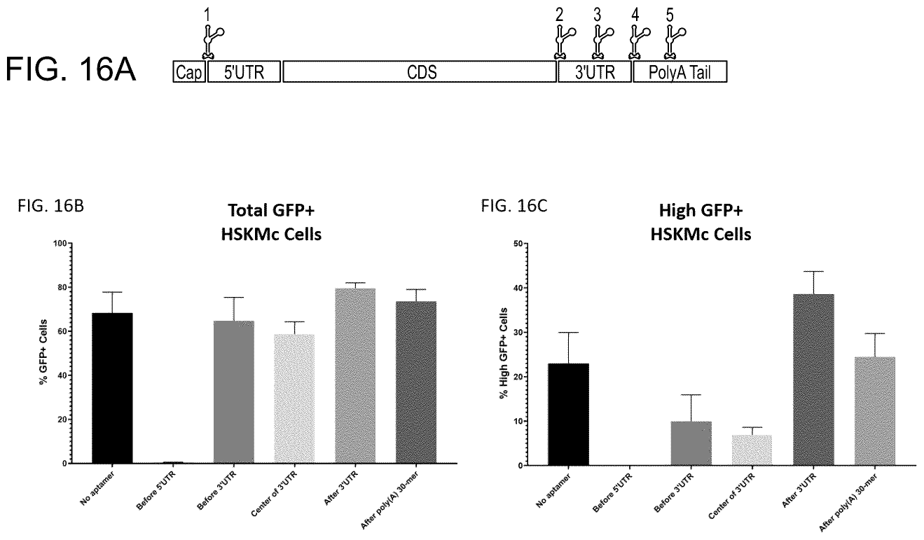

As shown in FIG. 16A-FIG. 16C and FIG. 17A-FIG. 17B, the mRNA translation efficiency for aptamer tagged mRNA where the aptamer varied in placement within the mRNA was assessed in either HskMc and Hela cell lines, respectively. Expression was quantified as the total number of cells with GFP signal above background (% GFP+ Cells), as well as the number of cells above a certain signal intensity threshold (% high GFP+ cells).

The location of the aptamer tag within the full-length mRNA sequence had a significant impact on translation efficiency. Placement of the aptamer at the 5′ end of the mRNA eliminated translation, while all other locations allowed for varying levels of translation. Positioning the aptamer after the 3′ UTR resulted in the highest translation efficiency as demonstrated by the increased GFP intensity. This trend was reproducible across both HskMc and Hela cell lines.

### Example 12: Analysis of Elongated polyA Tail Length on Translation Efficiency

Example 7 demonstrated that a longer polyA tail length increased translation efficiency of the aptamer tagged mRNA.

To quantify the amount of translational enhancement, elongated polyA tails were added to Sim aptamer tagged mRNA and tested in the mRNA translation efficiency assay described in Example 11. The vectors used for IVT included an encoded polyA tail, specifically a segmented polyA tail with 60 A's, a NsiI restriction enzyme cut site, then another 60 A's.

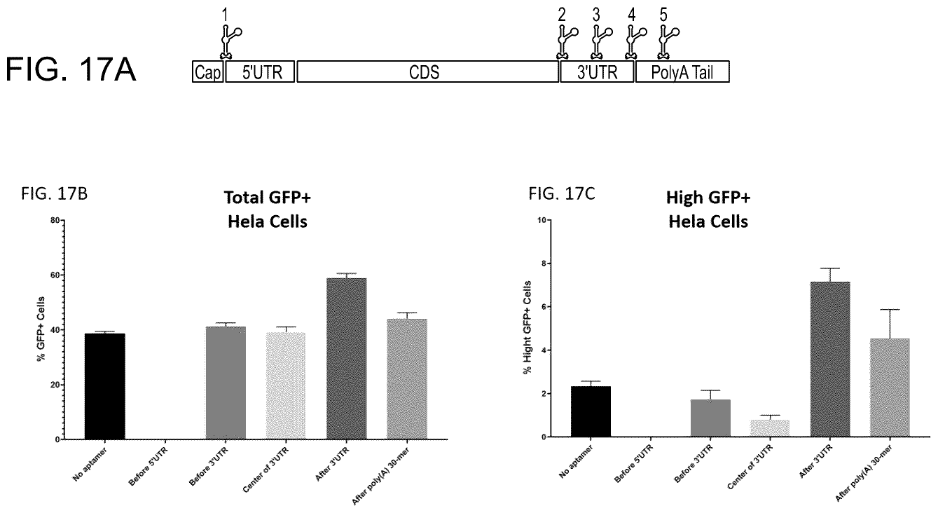

All mRNA produced from the vectors described above contained the segmented polyA tail and were ARCA capped. The two conditions on the right of FIG. 18 included an additional polyadenylation step where 1 μl of E. coli Poly (A) polymerase (NEB, M0276) was incubated for 45 minutes with buffer and additional ATP, which would typically add ˜200 A's to the end of each RNA shown in FIG. 18, the total number of GFP positive Hela cells (expressed as a percent) was significantly higher for the aptamer tagged mRNA with the elongated polyA tail relative to controls. This result confirms that elongating polyA tail lengths in an aptamer tagged mRNA can improve downstream mRNA translation kinetics in cells.

### Example 13: Analysis of mRNA Tagged with an Aptamer Embedded in RNA Scaffold on RNA Recovery and Translation Efficiency

To confirm and expand on the findings of Example 5, the Sim aptamer embedded in the tRNA scaffold tag (see Example 5) was compared to the 2×S1m and the 4×S1m aptamer tagged mRNA with respect to RNA recovery after streptavidin affinity purification and mRNA translation efficiency.

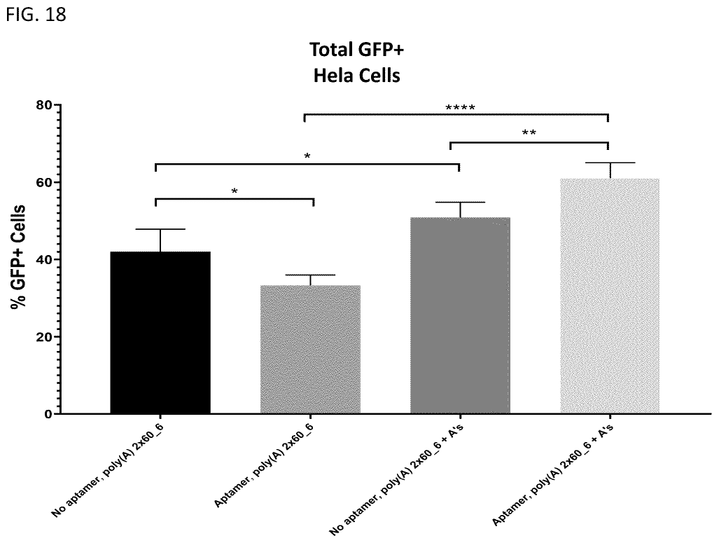

As shown in FIG. 19A, the addition of a stabilizing sequence surrounding the S1m aptamer resulted in RNA purification yields that were equal to the binding efficiency of the 4×S1m aptamer tagged mRNA, demonstrating that an RNA scaffold significantly increases affinity purification yield. Stabilization of the S1m aptamer with a tRNA scaffold had no impact on mRNA translation efficiency as shown in FIG. 19B. The results are summarized in Table 4 below.

TABLE 4
Percent unbound mRNA and percent eluted
mRNA for the data of FIG. 19A

| | Unbound | | Elution | |
|---|---|---|---|---|
| Plasmid used to | | Standard | | Standard |
| generate mRNA | Average | Deviation | Average | Deviation |
| pAM17 | 60.5% | 5.4% | 11.2% | 0.7% |
| (no aptamer) | | | | |
| pAM31 | 14.1% | 0.3% | 46.0% | 1.8% |
| (2xS1m) | | | | |
| pAM28 | 6.7% | 0.5% | 47.2% | 5.2% |
| (4xS1m) | | | | |
| pAM37 | 5.9% | 0.3% | 54.9% | 5.7% |
| (TRNA S1m) | | | | |

### Example 14: Synthesis and Affinity Purification of mRNA Tagged with Aptamer Stabilized in a Bioorthogonal RNA Scaffold

tRNA scaffolded aptamers often have reduced RNA stability due to endonucleolytic cleavage in bacterial and mammalian cells. Filonov et al. (2015) Chem Biol. 22 (5): 649-660. An alternative to tRNA scaffolds are bioorthogonal scaffolds. Bioorthogonal scaffolds are not readily recognized by intracellular nucleases and targeted for degradation, such as, the V5, the F29, or the F30 scaffold. Id.

To test whether bioorthogonal scaffolds could stabilize the S1m aptamer and improve efficiency in a downstream mRNA affinity purification process two vectors were constructed containing either the F30 scaffold stabilizing the 1×S1m aptamer (F30-1×S1m aptamer) or the F30 scaffold stabilizing the 2×S1m aptamer (F30-2×S1m aptamer). The F30-1×S1m aptamer and F30-2×S1m aptamer sequence are provided below.

DNA sequence encoding F30-1xS1m
aptamer (F30 segments underlined and bold):
(SEQ ID NO: 28)
TTGCCATGTGTATGTGGGATGCGGCCGCCGACCAGAATCATGCAA
GTGCGTAAGATAGTCGCGGGTCGGCGGCCGCATCCCACATACTCT
GATGATCCTTCGGGATCATTCATGGCAA
F30-1xS1m aptamer
(F30 segments underlined and bold):
(SEQ ID NO: 29)
UUGCCAUGUGUAUGUGGGAUGCGGCCGCCGACCAGAAUCAUGCAA
GUGCGUAAGAUAGUCGCGGGUCGGCGGCCGCAUCCCACAUACUCU
GAUGAUCCUUCGGGAUCAUUCAUGGCAA
DNA sequence encoding F30-2xS1m aptamer
(F30 segments underlined and bold):
(SEQ ID NO: 30)
TTGCCATGTGTATGTGGGATGCGGCCGCCGACCAGAATCATGCAA
GTGCGTAAGATAGTCGCGGGTCGGCGGCCGCATCCCACATACTCT
GATGATCCATGCGGCCGCCGACCAGAATCATGCAAGTGCGTAAGA
TAGTCGCGGGTCGGCGGCCGCATGGATCATTCATGGCAA
F30-2xS1m aptamer
(F30 segments underlined and bold):
(SEQ ID NO: 31)
UUGCCAUGUGUAUGUGGGAUGCGGCCGCCGACCAGAAUCAUGCAA
GUGCGUAAGAUAGUCGCGGGUCGGCGGCCGCAUCCCACAUACUCU
GAUGAUCCAUGCGGCCGCCGACCAGAAUCAUGCAAGUGCGUAAGA
UAGUCGCGGGUCGGCGGCCGCAUGGAUCAUUCAUGGCAA

Other aptamers of interest may be readily inserted into the F30 scaffold. In a 1× aptamer configuration, a left F30 sequence and a “1× right” F30 sequence flank the one aptamer. In a 2× aptamer configuration, a left F30 sequence and middle F30 sequence flank the first aptamer, and the middle F30 sequence and a “2× right” F30 sequence flank the second aptamer. A F30-1× aptamer and F30-2× aptamer sequence are provided below.

DNA sequence encoding F30-1x aptamer:
(left F30 sequence, SEQ ID NO: 32)
TTGCCATGTGTATGTGGG-
APTAMER SEQUENCE-
(“1x right” F30 sequence, SEQ ID NO: 33)
CCCACATACTCTGATGATCCTTCGGGATCATTCATGGCAA
F30-1x aptamer:
(left F30 sequence, SEQ ID NO: 34)
UUGCCAUGUGUAUGUGGG-
APTAMER SEQUENCE-
(“1x right” F30 sequence, SEQ ID NO: 35)
CCCACAUACUCUGAUGAUCCUUCGGGAUCAUUCAUGGCAA
DNA sequence encoding F30-2x aptamer:
(left F30 sequence, SEQ ID NO: 36)
TTGCCATGTGTATGTGGG-
APTAMER SEQUENCE-
(middle F30 sequence, SEQ ID NO: 37)
CCCACATACTCTGATGATCC-
APTAMER SEQUENCE-
(“2x right” F30 sequence, SEQ ID NO: 38)
GGATCATTCATGGCAA
F30-2xS1m aptamer
(F30 segments underlined and bold):
(left F30 sequence, SEQ ID NO: 39)
UUGCCAUGUGUAUGUGGG-
APTAMER SEQUENCE-
(middle F30 sequence, SEQ ID NO: 40)
CCCACAUACUCUGAUGAUCC-
APTAMER SEQUENCE-
(“2x right” F30 sequence, SEQ ID NO: 41)
GGAUCAUUCAUGGCAA

To analyze the affinity binding of the F30-1×S1m and the F30-2×S1m aptamer mRNA, the aptamer mRNA was affinity purified with streptavidin sepharose beads, eluted, and the amount of RNA recovery in the eluate was quantified using the methods described above. The binding affinity of streptavidin sepharose beads to either untagged mRNA (no aptamer control), the 4×S1m aptamer, the F30-1×S1m aptamer, or the F30-2×S1m aptamer tagged mRNA was evaluated and compared.

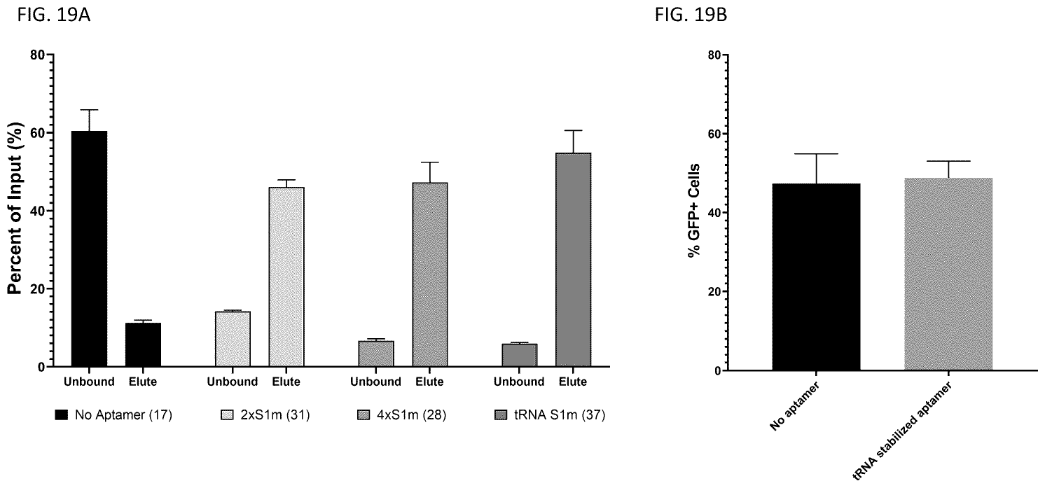

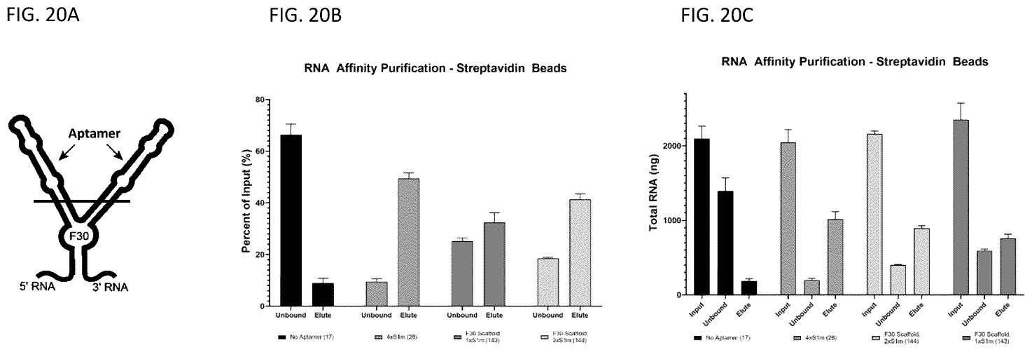

Affinity purified F30-1×S1m and the F30-2×S1m mRNA yielded about a 30-40% RNA recovery yield relative to the input samples collected prior to incubation with streptavidin beads (FIG. 20B) also shown in Table 5 below.

TABLE 5
Percent unbound mRNA and percent eluted
mRNA for the data of FIG. 20B

| Plasmid used to | | Standard | | Standard |
|---|---|---|---|---|
| generate mRNA | Average | Deviation | Average | Deviation |
| pAM17 | 66.4% | 4.2.% | 9.0% | 1.8% |
| (no aptamer) | | | | |
| pAM28 | 9.6% | 1.1% | 49.5% | 2.2% |
| (4xS1m) | | | | |
| pAM143 | 25.2% | 1.2% | 32.5% | 3.7% |
| F30-1xS1m) | | | | |
| pAM144 | 18.6% | 0.4% | 41.4% | 2.2% |
| F30-2xS1m) | | | | |

The total RNA recovery from the eluted F30-2×S1m and the F30-1×S1m tagged mRNA was approximately 900 ng/μL and 800 ng/μL, respectively (FIG. 20C). In contrast, the affinity purified eluted negative control yielded only 200 ng/μL of RNA recovery yield.

This result shows that introducing a bioorthogonal scaffold (i.e., F30) to stabilize an aptamer (e.g., the Sim aptamer) can potentially be used to improve the affinity purification efficiency of mRNA.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

### SEQUENCES

TABLE 6
mRNA transcript-encoding nucleotide sequences

| SEQ ID NO/ | |
|---|---|
| Description | SEQUENCE |
| SEQ ID NO: | TAATACGACTCACTATAGGAGAGCGGCCGCTTTTTCAGCAAGATT |
| 9 | AAGCCCAGGGCAGAGCCATCTATTGCTTACATTTGCTTCTGACAC |
| pAM17 | AACTGTGTTCACTAGCAACCTCAAACAGACACCATGGTGAGCAAG |
| No aptamer | GGCGAGGAGCTGTTCACCGGGGTGGTGCCCATCCTGGTCGAGCTG |
| control | GACGGCGACGTAAACGGCCACAAGTTCAGCGTGTCCGGCGAGGGC |
| | GAGGGCGATGCCACCTACGGCAAGCTGACCCTGAAGTTCATCTGC |
| | ACCACCGGCAAGCTGCCCGTGCCCTGGCCCACCCTCGTGACCACC |
| | CTGACCTACGGCGTGCAGTGCTTCAGCCGCTACCCCGACCACATG |
| | AAGCAGCACGACTTCTTCAAGTCCGCCATGCCCGAAGGCTACGTC |
| | CAGGAGCGCACCATCTTCTTCAAGGACGACGGCAACTACAAGACC |
| | CGCGCCGAGGTGAAGTTCGAGGGCGACACCCTGGTGAACCGCATC |
| | GAGCTGAAGGGCATCGACTTCAAGGAGGACGGCAACATCCTGGGG |
| | CACAAGCTGGAGTACAACTACAACAGCCACAACGTCTATATCATG |
| | GCCGACAAGCAGAAGAACGGCATCAAGGTGAACTTCAAGATCCGC |
| | CACAACATCGAGGACGGCAGCGTGCAGCTCGCCGACCACTACCAG |
| | CAGAACACCCCCATCGGCGACGGCCCCGTGCTGCTGCCCGACAAC |
| | CACTACCTGAGCACCCAGTCCGCCCTGAGCAAAGACCCCAACGAG |
| | AAGCGCGATCACATGGTCCTGCTGGAGTTCGTGACCGCCGCCGGG |
| | ATCACTCTCGGCATGGACGAGCTGTACAAGTAATAAAGCTCGCTT |
| | TCTTGCTGTCCAATTTCTATTAAAGGTTCCTTTGTTCCCTAAGTC |
| | CAACTACTAAACTGGGGGATATTATGAAGGGCCTTGAGCATCTGG |
| | ATTCTGCCTAATAAAAAACATTTATTTTCATTGCAGCTCGCTTTC |
| | TTGCTGTCCAATTTCTATTAAAGGTTCCTTTGTTCCCTAAGTCCA |
| | ACTACTAAACTGGGGGATATTATGAAGGGCCTTGAGCATCTGGAT |
| | TCTGCCTAATAAAAAACATTTATTTTCATTGCAAAAAAAAAAAAA |
| | AAAAAAAAAAAAAAAAAGAAGAGCCGTACGGGCGCGCCTAGGCGC |
| | GATTCCGCTTCCTCGCTCACTGACTCGCTGCGCTCGGTCGTTCGG |
| | CTGCGGCGAGCGGTATCAGCTCACTCAAAGGCGGTAATACGGTTA |
| | TCCACAGAATCAGGGGATAACGCAGGAAAGAACATGTGAGCAAAA |
| | GGCCAGCAAAAGGCCAGGAACCGTAAAAAGGCCGCGTTGCTGGCG |
| | TTTTTCCATAGGCTCCGCCCCCCTGACGAGCATCACAAAAATCGA |
| | CGCTCAAGTCAGAGGTGGCGAAACCCGACAGGACTATAAAGATAC |
| | CAGGCGTTTCCCCCTGGAAGCTCCCTCGTGCGCTCTCCTGTTCCG |
| | ACCCTGCCGCTTACCGGATACCTGTCCGCCTTTCTCCCTTCGGGA |
| | AGCGTGGCGCTTTCTCATAGCTCACGCTGTAGGTATCTCAGTTCG |
| | GTGTAGGTCGTTCGCTCCAAGCTGGGCTGTGTGCACGAACCCCCC |
| | GTTCAGCCCGACCGCTGCGCCTTATCCGGTAACTATCGTCTTGAG |
| | TCCAACCCGGTAAGACACGACTTATCGCCACTGGCAGCAGCCACT |
| | GGTAACAGGATTAGCAGAGCGAGGTATGTAGGCGGTGCTACAGAG |
| | TTCTTGAAGTGGTGGCCTAACTACGGCTACACTAGAAGAACAGTA |
| | TTTGGTATCTGCGCTCTGCTGAAGCCAGTTACCTTCGGAAAAAGA |
| | GTTGGTAGCTCTTGATCCGGCAAACAAACCACCGCTGGTAGCGGT |
| | GGTTTTTTTGTTTGCAAGCAGCAGATTACGCGCAGAAAAAAAGGA |
| | TCTCAAGAAGATCCTTTGATCTTTTCTACGGGGTCTGACGCTCAG |
| | TGGAACGAAAACTCACGTTAAGGGATTTTGGTCATGAGATTATCA |
| | AAAAGGATCTTCACCTAGATCCTTTTAAATTAAAAATGAAGTTTT |
| | AAATCAATCTAAAGTATATATGAGTAAACTTGGTCTGACAGTTAC |
| | CAATGCTTAATCAGTGAGGCACCTATCTCAGCGATCTGTCTATTT |
| | CGTTCATCCATAGTTGCCTGACTCCCCGTCGTGTAGATAACTACG |
| | ATACGGGAGGGCTTACCATCTGGCCCCAGTGCTGCAATGATACCG |
| | CGAGATCCACGCTCACCGGCTCCAGATTTATCAGCAATAAACCAG |
| | CCAGCCGGAAGGGCCGAGCGCAGAAGTGGTCCTGCAACTTTATCC |
| | GCCTCCATCCAGTCTATTAATTGTTGCCGGGAAGCTAGAGTAAGT |
| | AGTTCGCCAGTTAATAGTTTGCGCAACGTTGTTGCCATTGCTACA |
| | GGCATCGTGGTGTCACGCTCGTCGTTTGGTATGGCTTCATTCAGC |
| | TCCGGTTCCCAACGATCAAGGCGAGTTACATGATCCCCCATGTTG |
| | TGCAAAAAAGCGGTTAGCTCCTTCGGTCCTCCGATCGTTGTCAGA |
| | AGTAAGTTGGCCGCAGTGTTATCACTCATGGTTATGGCAGCACTG |
| | CATAATTCTCTTACTGTCATGCCATCCGTAAGATGCTTTTCTGTG |
| | ACTGGTGAGTACTCAACCAAGTCATTCTGAGAATAGTGTATGCGG |
| | CGACCGAGTTGCTCTTGCCCGGCGTCAATACGGGATAATACCGCG |
| | CCACATAGCAGAACTTTAAAAGTGCTCATCATTGGAAAACGTTCT |
| | TCGGGGCGAAAACTCTCAAGGATCTTACCGCTGTTGAGATCCAGT |
| | TCGATGTAACCCACTCGTGCACCCAACTGATCTTCAGCATCTTTT |
| | ACTTTCACCAGCGTTTCTGGGTGAGCAAAAACAGGAAGGCAAAAT |
| | GCCGCAAAAAAGGGAATAAGGGCGACACGGAAATGTTGAATACTC |
| | ATACTCTTCCTTTTTCAATATTATTGAAGCATTTATCAGGGTTAT |
| | TGTCTCATGAGCGGATACATATTTGAATGTATTTAGAAAAATAAA |
| | CAAATAGGGGTTCCGCGCACATTTCCCCGAAAAGTGCCACCTGAC |
| | GTCTAAGAAACCATTATTATCATGACATTAACCTATAAAAATAGG |
| | CGTATCACGAGGCCCTTTCGTC |
| SEQ ID NO: | TAATACGACTCACTATAGGGGATCCGTAGAAAATGCGGCCGCCGA |
| 10 | CCAGAATCATGCAAGTGCGTAAGATAGTCGCGGGTCGGCGGCCGC |
| pAM25 | ATCTGCTGGGAAGCTACGATCCGTAGAAAATGCGGCCGCCGACCA |
| 4×S1m | GAATCATGCAAGTGCGTAAGATAGTCGCGGGTCGGCGGCCGCATC |
| before 5′ | TGCTGGGTAGCTGTGAACCGTAGAAAATGCGGCCGCCGACCAGAA |
| UTR | TCATGCAAGTGCGTAAGATAGTCGCGGGTCGGCGGCCGCATCTGC |
| | TGGGAAGCTACGATCCGTAGAAAATGCGGCCGCCGACCAGAATCA |
| | TGCAAGTGCGTAAGATAGTCGCGGGTCGGCGGCCGCATCTGCTGG |
| | GAAGCTTAGAGCGGCCGCTTTTTCAGCAAGATTAAGCCCAGGGCA |
| | GAGCCATCTATTGCTTACATTTGCTTCTGACACAACTGTGTTCAC |
| | TAGCAACCTCAAACAGACACCATGGTGAGCAAGGGCGAGGAGCTG |
| | TTCACCGGGGTGGTGCCCATCCTGGTCGAGCTGGACGGCGACGTA |
| | AACGGCCACAAGTTCAGCGTGTCCGGCGAGGGCGAGGGCGATGCC |
| | ACCTACGGCAAGCTGACCCTGAAGTTCATCTGCACCACCGGCAAG |
| | CTGCCCGTGCCCTGGCCCACCCTCGTGACCACCCTGACCTACGGC |
| | GTGCAGTGCTTCAGCCGCTACCCCGACCACATGAAGCAGCACGAC |
| | TTCTTCAAGTCCGCCATGCCCGAAGGCTACGTCCAGGAGCGCACC |
| | ATCTTCTTCAAGGACGACGGCAACTACAAGACCCGCGCCGAGGTG |
| | AAGTTCGAGGGCGACACCCTGGTGAACCGCATCGAGCTGAAGGGC |
| | ATCGACTTCAAGGAGGACGGCAACATCCTGGGGCACAAGCTGGAG |
| | TACAACTACAACAGCCACAACGTCTATATCATGGCCGACAAGCAG |
| | AAGAACGGCATCAAGGTGAACTTCAAGATCCGCCACAACATCGAG |
| | GACGGCAGCGTGCAGCTCGCCGACCACTACCAGCAGAACACCCCC |
| | ATCGGCGACGGCCCCGTGCTGCTGCCCGACAACCACTACCTGAGC |
| | ACCCAGTCCGCCCTGAGCAAAGACCCCAACGAGAAGCGCGATCAC |
| | ATGGTCCTGCTGGAGTTCGTGACCGCCGCCGGGATCACTCTCGGC |
| | ATGGACGAGCTGTACAAGTAATAAAGCTCGCTTTCTTGCTGTCCA |
| | ATTTCTATTAAAGGTTCCTTTGTTCCCTAAGTCCAACTACTAAAC |
| | TGGGGGATATTATGAAGGGCCTTGAGCATCTGGATTCTGCCTAAT |
| | AAAAAACATTTATTTTCATTGCAGCTCGCTTTCTTGCTGTCCAAT |
| | TTCTATTAAAGGTTCCTTTGTTCCCTAAGTCCAACTACTAAACTG |
| | GGGGATATTATGAAGGGCCTTGAGCATCTGGATTCTGCCTAATAA |
| | AAAACATTTATTTTCATTGCAAAAAAAAAAAAAAAAAAAAAAAAA |
| | AAAAAGAAGAGCCGTACGGGCGCGCCTAGGCGCGATTCCGCTTCC |
| | TCGCTCACTGACTCGCTGCGCTCGGTCGTTCGGCTGCGGCGAGCG |
| | GTATCAGCTCACTCAAAGGCGGTAATACGGTTATCCACAGAATCA |
| | GGGGATAACGCAGGAAAGAACATGTGAGCAAAAGGCCAGCAAAAG |
| | GCCAGGAACCGTAAAAAGGCCGCGTTGCTGGCGTTTTTCCATAGG |
| | CTCCGCCCCCCTGACGAGCATCACAAAAATCGACGCTCAAGTCAG |
| | AGGTGGCGAAACCCGACAGGACTATAAAGATACCAGGCGTTTCCC |
| | CCTGGAAGCTCCCTCGTGCGCTCTCCTGTTCCGACCCTGCCGCTT |
| | ACCGGATACCTGTCCGCCTTTCTCCCTTCGGGAAGCGTGGCGCTT |
| | TCTCATAGCTCACGCTGTAGGTATCTCAGTTCGGTGTAGGTCGTT |
| | CGCTCCAAGCTGGGCTGTGTGCACGAACCCCCCGTTCAGCCCGAC |
| | CGCTGCGCCTTATCCGGTAACTATCGTCTTGAGTCCAACCCGGTA |
| | AGACACGACTTATCGCCACTGGCAGCAGCCACTGGTAACAGGATT |
| | AGCAGAGCGAGGTATGTAGGCGGTGCTACAGAGTTCTTGAAGTGG |
| | TGGCCTAACTACGGCTACACTAGAAGAACAGTATTTGGTATCTGC |
| | GCTCTGCTGAAGCCAGTTACCTTCGGAAAAAGAGTTGGTAGCTCT |
| | TGATCCGGCAAACAAACCACCGCTGGTAGCGGTGGTTTTTTTGTT |
| | TGCAAGCAGCAGATTACGCGCAGAAAAAAAGGATCTCAAGAAGAT |
| | CCTTTGATCTTTTCTACGGGGTCTGACGCTCAGTGGAACGAAAAC |
| | TCACGTTAAGGGATTTTGGTCATGAGATTATCAAAAAGGATCTTC |
| | ACCTAGATCCTTTTAAATTAAAAATGAAGTTTTAAATCAATCTAA |
| | AGTATATATGAGTAAACTTGGTCTGACAGTTACCAATGCTTAATC |
| | AGTGAGGCACCTATCTCAGCGATCTGTCTATTTCGTTCATCCATA |
| | GTTGCCTGACTCCCCGTCGTGTAGATAACTACGATACGGGAGGGC |
| | TTACCATCTGGCCCCAGTGCTGCAATGATACCGCGAGATCCACGC |
| | TCACCGGCTCCAGATTTATCAGCAATAAACCAGCCAGCCGGAAGG |
| | GCCGAGCGCAGAAGTGGTCCTGCAACTTTATCCGCCTCCATCCAG |
| | TCTATTAATTGTTGCCGGGAAGCTAGAGTAAGTAGTTCGCCAGTT |
| | AATAGTTTGCGCAACGTTGTTGCCATTGCTACAGGCATCGTGGTG |
| | TCACGCTCGTCGTTTGGTATGGCTTCATTCAGCTCCGGTTCCCAA |
| | CGATCAAGGCGAGTTACATGATCCCCCATGTTGTGCAAAAAAGCG |
| | GTTAGCTCCTTCGGTCCTCCGATCGTTGTCAGAAGTAAGTTGGCC |
| | GCAGTGTTATCACTCATGGTTATGGCAGCACTGCATAATTCTCTT |
| | ACTGTCATGCCATCCGTAAGATGCTTTTCTGTGACTGGTGAGTAC |
| | TCAACCAAGTCATTCTGAGAATAGTGTATGCGGCGACCGAGTTGC |
| | TCTTGCCCGGCGTCAATACGGGATAATACCGCGCCACATAGCAGA |
| | ACTTTAAAAGTGCTCATCATTGGAAAACGTTCTTCGGGGCGAAAA |
| | CTCTCAAGGATCTTACCGCTGTTGAGATCCAGTTCGATGTAACCC |
| | ACTCGTGCACCCAACTGATCTTCAGCATCTTTTACTTTCACCAGC |
| | GTTTCTGGGTGAGCAAAAACAGGAAGGCAAAATGCCGCAAAAAAG |
| | GGAATAAGGGCGACACGGAAATGTTGAATACTCATACTCTTCCTT |
| | TTTCAATATTATTGAAGCATTTATCAGGGTTATTGTCTCATGAGC |
| | GGATACATATTTGAATGTATTTAGAAAAATAAACAAATAGGGGTT |
| | CCGCGCACATTTCCCCGAAAAGTGCCACCTGACGTCTAAGAAACC |
| SEQ ID NO: | ATTATTATCATGACATTAACCTATAAAAATAGGCGTATCACGAGG |
| 11 | CCCTTTCGTCTAATACGACTCACTATAGGAGAGCGGCCGCTTTTT |
| pAM26 | CAGCAAGATTAAGCCCAGGGCAGAGCCATCTATTGCTTACATTTG |
| 4xS1m | CTTCTGACACAACTGTGTTCACTAGCAACCTCAAACAGACACCAT |
| Before | GGTGAGCAAGGGCGAGGAGCTGTTCACCGGGGTGGTGCCCATCCT |
| 3′UTR | GGTCGAGCTGGACGGCGACGTAAACGGCCACAAGTTCAGCGTGTC |
| | CGGCGAGGGCGAGGGCGATGCCACCTACGGCAAGCTGACCCTGAA |
| | GTTCATCTGCACCACCGGCAAGCTGCCCGTGCCCTGGCCCACCCT |
| | CGTGACCACCCTGACCTACGGCGTGCAGTGCTTCAGCCGCTACCC |
| | CGACCACATGAAGCAGCACGACTTCTTCAAGTCCGCCATGCCCGA |
| | AGGCTACGTCCAGGAGCGCACCATCTTCTTCAAGGACGACGGCAA |
| | CTACAAGACCCGCGCCGAGGTGAAGTTCGAGGGCGACACCCTGGT |
| | GAACCGCATCGAGCTGAAGGGCATCGACTTCAAGGAGGACGGCAA |
| | CATCCTGGGGCACAAGCTGGAGTACAACTACAACAGCCACAACGT |
| | CTATATCATGGCCGACAAGCAGAAGAACGGCATCAAGGTGAACTT |
| | CAAGATCCGCCACAACATCGAGGACGGCAGCGTGCAGCTCGCCGA |
| | CCACTACCAGCAGAACACCCCCATCGGCGACGGCCCCGTGCTGCT |
| | GCCCGACAACCACTACCTGAGCACCCAGTCCGCCCTGAGCAAAGA |
| | CCCCAACGAGAAGCGCGATCACATGGTCCTGCTGGAGTTCGTGAC |
| | CGCCGCCGGGATCACTCTCGGCATGGACGAGCTGTACAAGTAATA |
| | AGGATCCGTAGAAAATGCGGCCGCCGACCAGAATCATGCAAGTGC |
| | GTAAGATAGTCGCGGGTCGGCGGCCGCATCTGCTGGGAAGCTACG |
| | ATCCGTAGAAAATGCGGCCGCCGACCAGAATCATGCAAGTGCGTA |
| | AGATAGTCGCGGGTCGGCGGCCGCATCTGCTGGGTAGCTGTGAAC |
| | CGTAGAAAATGCGGCCGCCGACCAGAATCATGCAAGTGCGTAAGA |
| | TAGTCGCGGGTCGGCGGCCGCATCTGCTGGGAAGCTACGATCCGT |
| | AGAAAATGCGGCCGCCGACCAGAATCATGCAAGTGCGTAAGATAG |
| | TCGCGGGTCGGCGGCCGCATCTGCTGGGAAGCTTAGCTCGCTTTC |
| | TTGCTGTCCAATTTCTATTAAAGGTTCCTTTGTTCCCTAAGTCCA |
| | ACTACTAAACTGGGGGATATTATGAAGGGCCTTGAGCATCTGGAT |
| | TCTGCCTAATAAAAAACATTTATTTTCATTGCAGCTCGCTTTCTT |
| | GCTGTCCAATTTCTATTAAAGGTTCCTTTGTTCCCTAAGTCCAAC |
| | TACTAAACTGGGGGATATTATGAAGGGCCTTGAGCATCTGGATTC |
| | TGCCTAATAAAAAACATTTATTTTCATTGCAAAAAAAAAAAAAAA |
| | AAAAAAAAAAAAAAAGAAGAGCCGTACGGGCGCGCCTAGGCGCGA |
| | TTCCGCTTCCTCGCTCACTGACTCGCTGCGCTCGGTCGTTCGGCT |
| | GCGGCGAGCGGTATCAGCTCACTCAAAGGCGGTAATACGGTTATC |
| | CACAGAATCAGGGGATAACGCAGGAAAGAACATGTGAGCAAAAGG |
| | CCAGCAAAAGGCCAGGAACCGTAAAAAGGCCGCGTTGCTGGCGTT |
| | TTTCCATAGGCTCCGCCCCCCTGACGAGCATCACAAAAATCGACG |
| | CTCAAGTCAGAGGTGGCGAAACCCGACAGGACTATAAAGATACCA |
| | GGCGTTTCCCCCTGGAAGCTCCCTCGTGCGCTCTCCTGTTCCGAC |
| | CCTGCCGCTTACCGGATACCTGTCCGCCTTTCTCCCTTCGGGAAG |
| | CGTGGCGCTTTCTCATAGCTCACGCTGTAGGTATCTCAGTTCGGT |
| | GTAGGTCGTTCGCTCCAAGCTGGGCTGTGTGCACGAACCCCCCGT |
| | TCAGCCCGACCGCTGCGCCTTATCCGGTAACTATCGTCTTGAGTC |
| | CAACCCGGTAAGACACGACTTATCGCCACTGGCAGCAGCCACTGG |
| | TAACAGGATTAGCAGAGCGAGGTATGTAGGCGGTGCTACAGAGTT |
| | CTTGAAGTGGTGGCCTAACTACGGCTACACTAGAAGAACAGTATT |
| | TGGTATCTGCGCTCTGCTGAAGCCAGTTACCTTCGGAAAAAGAGT |
| | TGGTAGCTCTTGATCCGGCAAACAAACCACCGCTGGTAGCGGTGG |
| | TTTTTTTGTTTGCAAGCAGCAGATTACGCGCAGAAAAAAAGGATC |
| | TCAAGAAGATCCTTTGATCTTTTCTACGGGGTCTGACGCTCAGTG |
| | GAACGAAAACTCACGTTAAGGGATTTTGGTCATGAGATTATCAAA |
| | AAGGATCTTCACCTAGATCCTTTTAAATTAAAAATGAAGTTTTAA |
| | ATCAATCTAAAGTATATATGAGTAAACTTGGTCTGACAGTTACCA |
| | ATGCTTAATCAGTGAGGCACCTATCTCAGCGATCTGTCTATTTCG |
| | TTCATCCATAGTTGCCTGACTCCCCGTCGTGTAGATAACTACGAT |
| | ACGGGAGGGCTTACCATCTGGCCCCAGTGCTGCAATGATACCGCG |
| | AGATCCACGCTCACCGGCTCCAGATTTATCAGCAATAAACCAGCC |
| | AGCCGGAAGGGCCGAGCGCAGAAGTGGTCCTGCAACTTTATCCGC |
| | CTCCATCCAGTCTATTAATTGTTGCCGGGAAGCTAGAGTAAGTAG |
| | TTCGCCAGTTAATAGTTTGCGCAACGTTGTTGCCATTGCTACAGG |
| | CATCGTGGTGTCACGCTCGTCGTTTGGTATGGCTTCATTCAGCTC |
| | CGGTTCCCAACGATCAAGGCGAGTTACATGATCCCCCATGTTGTG |
| | CAAAAAAGCGGTTAGCTCCTTCGGTCCTCCGATCGTTGTCAGAAG |
| | TAAGTTGGCCGCAGTGTTATCACTCATGGTTATGGCAGCACTGCA |
| | TAATTCTCTTACTGTCATGCCATCCGTAAGATGCTTTTCTGTGAC |
| | TGGTGAGTACTCAACCAAGTCATTCTGAGAATAGTGTATGCGGCG |
| | ACCGAGTTGCTCTTGCCCGGCGTCAATACGGGATAATACCGCGCC |
| | ACATAGCAGAACTTTAAAAGTGCTCATCATTGGAAAACGTTCTTC |
| | GGGGCGAAAACTCTCAAGGATCTTACCGCTGTTGAGATCCAGTTC |
| | GATGTAACCCACTCGTGCACCCAACTGATCTTCAGCATCTTTTAC |
| | TTTCACCAGCGTTTCTGGGTGAGCAAAAACAGGAAGGCAAAATGC |
| | CGCAAAAAAGGGAATAAGGGCGACACGGAAATGTTGAATACTCAT |
| | ACTCTTCCTTTTTCAATATTATTGAAGCATTTATCAGGGTTATTG |
| | TCTCATGAGCGGATACATATTTGAATGTATTTAGAAAAATAAACA |
| | AATAGGGGTTCCGCGCACATTTCCCCGAAAAGTGCCACCTGACGT |
| | CTAAGAAACCATTATTATCATGACATTAACCTATAAAAATAGGCG |
| | TATCACGAGGCCCTTTCGTC |
| SEQ ID NO: | TAATACGACTCACTATAGGAGAGCGGCCGCTTTTTCAGCAAGATT |
| 12 | AAGCCCAGGGCAGAGCCATCTATTGCTTACATTTGCTTCTGACAC |
| pAM27 | AACTGTGTTCACTAGCAACCTCAAACAGACACCATGGTGAGCAAG |
| 4xS1m | GGCGAGGAGCTGTTCACCGGGGTGGTGCCCATCCTGGTCGAGCTG |
| bisecting 3′ | GACGGCGACGTAAACGGCCACAAGTTCAGCGTGTCCGGCGAGGGC |
| UTR | GAGGGCGATGCCACCTACGGCAAGCTGACCCTGAAGTTCATCTGC |
| | ACCACCGGCAAGCTGCCCGTGCCCTGGCCCACCCTCGTGACCACC |
| | CTGACCTACGGCGTGCAGTGCTTCAGCCGCTACCCCGACCACATG |
| | AAGCAGCACGACTTCTTCAAGTCCGCCATGCCCGAAGGCTACGTC |
| | CAGGAGCGCACCATCTTCTTCAAGGACGACGGCAACTACAAGACC |
| | CGCGCCGAGGTGAAGTTCGAGGGCGACACCCTGGTGAACCGCATC |
| | GAGCTGAAGGGCATCGACTTCAAGGAGGACGGCAACATCCTGGGG |
| | CACAAGCTGGAGTACAACTACAACAGCCACAACGTCTATATCATG |
| | GCCGACAAGCAGAAGAACGGCATCAAGGTGAACTTCAAGATCCGC |
| | CACAACATCGAGGACGGCAGCGTGCAGCTCGCCGACCACTACCAG |
| | CAGAACACCCCCATCGGCGACGGCCCCGTGCTGCTGCCCGACAAC |
| | CACTACCTGAGCACCCAGTCCGCCCTGAGCAAAGACCCCAACGAG |
| | AAGCGCGATCACATGGTCCTGCTGGAGTTCGTGACCGCCGCCGGG |
| | ATCACTCTCGGCATGGACGAGCTGTACAAGTAATAAAGCTCGCTT |
| | TCTTGCTGTCCAATTTCTATTAAAGGTTCCTTTGTTCCCTAAGTC |
| | CAACTACTAAACTGGGGGATATTATGAAGGGCCTTGAGCATCTGG |
| | ATTCTGCCTAATAAAAAACATTTATTTTCATTGCGGATCCGTAGA |
| | AAATGCGGCCGCCGACCAGAATCATGCAAGTGCGTAAGATAGTCG |
| | CGGGTCGGCGGCCGCATCTGCTGGGAAGCTACGATCCGTAGAAAA |
| | TGCGGCCGCCGACCAGAATCATGCAAGTGCGTAAGATAGTCGCGG |
| | GTCGGCGGCCGCATCTGCTGGGTAGCTGTGAACCGTAGAAAATGC |
| | GGCCGCCGACCAGAATCATGCAAGTGCGTAAGATAGTCGCGGGTC |
| | GGCGGCCGCATCTGCTGGGAAGCTACGATCCGTAGAAAATGCGGC |
| | CGCCGACCAGAATCATGCAAGTGCGTAAGATAGTCGCGGGTCGGC |
| | GGCCGCATCTGCTGGGAAGCTTAGCTCGCTTTCTTGCTGTCCAAT |
| | TTCTATTAAAGGTTCCTTTGTTCCCTAAGTCCAACTACTAAACTG |
| | GGGGATATTATGAAGGGCCTTGAGCATCTGGATTCTGCCTAATAA |
| | AAAACATTTATTTTCATTGCAAAAAAAAAAAAAAAAAAAAAAAAA |
| | AAAAAGAAGAGCCGTACGGGCGCGCCTAGGCGCGATTCCGCTTCC |
| | TCGCTCACTGACTCGCTGCGCTCGGTCGTTCGGCTGCGGCGAGCG |
| | GTATCAGCTCACTCAAAGGCGGTAATACGGTTATCCACAGAATCA |
| | GGGGATAACGCAGGAAAGAACATGTGAGCAAAAGGCCAGCAAAAG |
| | GCCAGGAACCGTAAAAAGGCCGCGTTGCTGGCGTTTTTCCATAGG |
| | CTCCGCCCCCCTGACGAGCATCACAAAAATCGACGCTCAAGTCAG |
| | AGGTGGCGAAACCCGACAGGACTATAAAGATACCAGGCGTTTCCC |
| | CCTGGAAGCTCCCTCGTGCGCTCTCCTGTTCCGACCCTGCCGCTT |
| | ACCGGATACCTGTCCGCCTTTCTCCCTTCGGGAAGCGTGGCGCTT |
| | TCTCATAGCTCACGCTGTAGGTATCTCAGTTCGGTGTAGGTCGTT |
| | CGCTCCAAGCTGGGCTGTGTGCACGAACCCCCCGTTCAGCCCGAC |
| | CGCTGCGCCTTATCCGGTAACTATCGTCTTGAGTCCAACCCGGTA |
| | AGACACGACTTATCGCCACTGGCAGCAGCCACTGGTAACAGGATT |
| | AGCAGAGCGAGGTATGTAGGCGGTGCTACAGAGTTCTTGAAGTGG |
| | TGGCCTAACTACGGCTACACTAGAAGAACAGTATTTGGTATCTGC |
| | GCTCTGCTGAAGCCAGTTACCTTCGGAAAAAGAGTTGGTAGCTCT |
| | TGATCCGGCAAACAAACCACCGCTGGTAGCGGTGGTTTTTTTGTT |
| | TGCAAGCAGCAGATTACGCGCAGAAAAAAAGGATCTCAAGAAGAT |
| | CCTTTGATCTTTTCTACGGGGTCTGACGCTCAGTGGAACGAAAAC |
| | TCACGTTAAGGGATTTTGGTCATGAGATTATCAAAAAGGATCTTC |
| | ACCTAGATCCTTTTAAATTAAAAATGAAGTTTTAAATCAATCTAA |
| | AGTATATATGAGTAAACTTGGTCTGACAGTTACCAATGCTTAATC |
| | AGTGAGGCACCTATCTCAGCGATCTGTCTATTTCGTTCATCCATA |
| | GTTGCCTGACTCCCCGTCGTGTAGATAACTACGATACGGGAGGGC |
| | TTACCATCTGGCCCCAGTGCTGCAATGATACCGCGAGATCCACGC |
| | TCACCGGCTCCAGATTTATCAGCAATAAACCAGCCAGCCGGAAGG |
| | GCCGAGCGCAGAAGTGGTCCTGCAACTTTATCCGCCTCCATCCAG |
| | TCTATTAATTGTTGCCGGGAAGCTAGAGTAAGTAGTTCGCCAGTT |
| | AATAGTTTGCGCAACGTTGTTGCCATTGCTACAGGCATCGTGGTG |
| | TCACGCTCGTCGTTTGGTATGGCTTCATTCAGCTCCGGTTCCCAA |
| | CGATCAAGGCGAGTTACATGATCCCCCATGTTGTGCAAAAAAGCG |
| | GTTAGCTCCTTCGGTCCTCCGATCGTTGTCAGAAGTAAGTTGGCC |
| | GCAGTGTTATCACTCATGGTTATGGCAGCACTGCATAATTCTCTT |
| | ACTGTCATGCCATCCGTAAGATGCTTTTCTGTGACTGGTGAGTAC |
| | TCAACCAAGTCATTCTGAGAATAGTGTATGCGGCGACCGAGTTGC |
| | TCTTGCCCGGCGTCAATACGGGATAATACCGCGCCACATAGCAGA |
| | ACTTTAAAAGTGCTCATCATTGGAAAACGTTCTTCGGGGCGAAAA |
| | CTCTCAAGGATCTTACCGCTGTTGAGATCCAGTTCGATGTAACCC |
| | ACTCGTGCACCCAACTGATCTTCAGCATCTTTTACTTTCACCAGC |
| | GTTTCTGGGTGAGCAAAAACAGGAAGGCAAAATGCCGCAAAAAAG |
| | GGAATAAGGGCGACACGGAAATGTTGAATACTCATACTCTTCCTT |
| | TTTCAATATTATTGAAGCATTTATCAGGGTTATTGTCTCATGAGC |
| | GGATACATATTTGAATGTATTTAGAAAAATAAACAAATAGGGGTT |
| | CCGCGCACATTTCCCCGAAAAGTGCCACCTGACGTCTAAGAAACC |
| | ATTATTATCATGACATTAACCTATAAAAATAGGCGTATCACGAGG |
| | CCCTTTCGTC |
| SEQ ID NO: | TAATACGACTCACTATAGGAGAGCGGCCGCTTTTTCAGCAAGATT |
| 13 | AAGCCCAGGGCAGAGCCATCTATTGCTTACATTTGCTTCTGACAC |
| pAM28 | AACTGTGTTCACTAGCAACCTCAAACAGACACCATGGTGAGCAAG |
| 4xS1m after | GGCGAGGAGCTGTTCACCGGGGTGGTGCCCATCCTGGTCGAGCTG |
| 3′ UTR | GACGGCGACGTAAACGGCCACAAGTTCAGCGTGTCCGGCGAGGGC |
| | GAGGGCGATGCCACCTACGGCAAGCTGACCCTGAAGTTCATCTGC |
| | ACCACCGGCAAGCTGCCCGTGCCCTGGCCCACCCTCGTGACCACC |
| | CTGACCTACGGCGTGCAGTGCTTCAGCCGCTACCCCGACCACATG |
| | AAGCAGCACGACTTCTTCAAGTCCGCCATGCCCGAAGGCTACGTC |
| | CAGGAGCGCACCATCTTCTTCAAGGACGACGGCAACTACAAGACC |
| | CGCGCCGAGGTGAAGTTCGAGGGCGACACCCTGGTGAACCGCATC |
| | GAGCTGAAGGGCATCGACTTCAAGGAGGACGGCAACATCCTGGGG |
| | CACAAGCTGGAGTACAACTACAACAGCCACAACGTCTATATCATG |
| | GCCGACAAGCAGAAGAACGGCATCAAGGTGAACTTCAAGATCCGC |
| | CACAACATCGAGGACGGCAGCGTGCAGCTCGCCGACCACTACCAG |
| | CAGAACACCCCCATCGGCGACGGCCCCGTGCTGCTGCCCGACAAC |
| | CACTACCTGAGCACCCAGTCCGCCCTGAGCAAAGACCCCAACGAG |
| | AAGCGCGATCACATGGTCCTGCTGGAGTTCGTGACCGCCGCCGGG |
| | ATCACTCTCGGCATGGACGAGCTGTACAAGTAATAAAGCTCGCTT |
| | TCTTGCTGTCCAATTTCTATTAAAGGTTCCTTTGTTCCCTAAGTC |
| | CAACTACTAAACTGGGGGATATTATGAAGGGCCTTGAGCATCTGG |
| | ATTCTGCCTAATAAAAAACATTTATTTTCATTGCAGCTCGCTTTC |
| | TTGCTGTCCAATTTCTATTAAAGGTTCCTTTGTTCCCTAAGTCCA |
| | ACTACTAAACTGGGGGATATTATGAAGGGCCTTGAGCATCTGGAT |
| | TCTGCCTAATAAAAAACATTTATTTTCATTGCGGATCCGTAGAAA |
| | ATGCGGCCGCCGACCAGAATCATGCAAGTGCGTAAGATAGTCGCG |
| | GGTCGGCGGCCGCATCTGCTGGGAAGCTACGATCCGTAGAAAATG |
| | CGGCCGCCGACCAGAATCATGCAAGTGCGTAAGATAGTCGCGGGT |
| | CGGCGGCCGCATCTGCTGGGTAGCTGTGAACCGTAGAAAATGCGG |
| | CCGCCGACCAGAATCATGCAAGTGCGTAAGATAGTCGCGGGTCGG |
| | CGGCCGCATCTGCTGGGAAGCTACGATCCGTAGAAAATGCGGCCG |
| | CCGACCAGAATCATGCAAGTGCGTAAGATAGTCGCGGGTCGGCGG |
| | CCGCATCTGCTGGGAAGCTTAAAAAAAAAAAAAAAAAAAAAAAAA |
| | AAAAAGAAGAGCCGTACGGGCGCGCCTAGGCGCGATTCCGCTTCC |
| | TCGCTCACTGACTCGCTGCGCTCGGTCGTTCGGCTGCGGCGAGCG |
| | GTATCAGCTCACTCAAAGGCGGTAATACGGTTATCCACAGAATCA |
| | GGGGATAACGCAGGAAAGAACATGTGAGCAAAAGGCCAGCAAAAG |
| | GCCAGGAACCGTAAAAAGGCCGCGTTGCTGGCGTTTTTCCATAGG |
| | CTCCGCCCCCCTGACGAGCATCACAAAAATCGACGCTCAAGTCAG |
| | AGGTGGCGAAACCCGACAGGACTATAAAGATACCAGGCGTTTCCC |
| | CCTGGAAGCTCCCTCGTGCGCTCTCCTGTTCCGACCCTGCCGCTT |
| | ACCGGATACCTGTCCGCCTTTCTCCCTTCGGGAAGCGTGGCGCTT |
| | TCTCATAGCTCACGCTGTAGGTATCTCAGTTCGGTGTAGGTCGTT |
| | CGCTCCAAGCTGGGCTGTGTGCACGAACCCCCCGTTCAGCCCGAC |
| | CGCTGCGCCTTATCCGGTAACTATCGTCTTGAGTCCAACCCGGTA |
| | AGACACGACTTATCGCCACTGGCAGCAGCCACTGGTAACAGGATT |
| | AGCAGAGCGAGGTATGTAGGCGGTGCTACAGAGTTCTTGAAGTGG |
| | TGGCCTAACTACGGCTACACTAGAAGAACAGTATTTGGTATCTGC |
| | GCTCTGCTGAAGCCAGTTACCTTCGGAAAAAGAGTTGGTAGCTCT |
| | TGATCCGGCAAACAAACCACCGCTGGTAGCGGTGGTTTTTTTGTT |
| | TGCAAGCAGCAGATTACGCGCAGAAAAAAAGGATCTCAAGAAGAT |
| | CCTTTGATCTTTTCTACGGGGTCTGACGCTCAGTGGAACGAAAAC |
| | TCACGTTAAGGGATTTTGGTCATGAGATTATCAAAAAGGATCTTC |
| | ACCTAGATCCTTTTAAATTAAAAATGAAGTTTTAAATCAATCTAA |
| | AGTATATATGAGTAAACTTGGTCTGACAGTTACCAATGCTTAATC |
| | AGTGAGGCACCTATCTCAGCGATCTGTCTATTTCGTTCATCCATA |
| | GTTGCCTGACTCCCCGTCGTGTAGATAACTACGATACGGGAGGGC |
| | TTACCATCTGGCCCCAGTGCTGCAATGATACCGCGAGATCCACGC |
| | TCACCGGCTCCAGATTTATCAGCAATAAACCAGCCAGCCGGAAGG |
| | GCCGAGCGCAGAAGTGGTCCTGCAACTTTATCCGCCTCCATCCAG |
| | TCTATTAATTGTTGCCGGGAAGCTAGAGTAAGTAGTTCGCCAGTT |
| | AATAGTTTGCGCAACGTTGTTGCCATTGCTACAGGCATCGTGGTG |
| | TCACGCTCGTCGTTTGGTATGGCTTCATTCAGCTCCGGTTCCCAA |
| | CGATCAAGGCGAGTTACATGATCCCCCATGTTGTGCAAAAAAGCG |
| | GTTAGCTCCTTCGGTCCTCCGATCGTTGTCAGAAGTAAGTTGGCC |
| | GCAGTGTTATCACTCATGGTTATGGCAGCACTGCATAATTCTCTT |
| | ACTGTCATGCCATCCGTAAGATGCTTTTCTGTGACTGGTGAGTAC |
| | TCAACCAAGTCATTCTGAGAATAGTGTATGCGGCGACCGAGTTGC |
| | TCTTGCCCGGCGTCAATACGGGATAATACCGCGCCACATAGCAGA |
| | ACTTTAAAAGTGCTCATCATTGGAAAACGTTCTTCGGGGCGAAAA |
| | CTCTCAAGGATCTTACCGCTGTTGAGATCCAGTTCGATGTAACCC |
| | ACTCGTGCACCCAACTGATCTTCAGCATCTTTTACTTTCACCAGC |
| | GTTTCTGGGTGAGCAAAAACAGGAAGGCAAAATGCCGCAAAAAAG |
| | GGAATAAGGGCGACACGGAAATGTTGAATACTCATACTCTTCCTT |
| | TTTCAATATTATTGAAGCATTTATCAGGGTTATTGTCTCATGAGC |
| | GGATACATATTTGAATGTATTTAGAAAAATAAACAAATAGGGGTT |
| | CCGCGCACATTTCCCCGAAAAGTGCCACCTGACGTCTAAGAAACC |
| | ATTATTATCATGACATTAACCTATAAAAATAGGCGTATCACGAGG |
| | CCCTTTCGTCTAATACGACTCACTATAGGAGAGCGGCCGCTTTTT |
| | CAGCAAGATTAAGCCCAGGGCAGAGCCATCTATTGCTTACATTTG |
| | CTTCTGACACAACTGTGTTCACTAGCAACCTCAAACAGACACCAT |
| | GGTGAGCAAGGGCGAGGAGCTGTTCACCGGGGTGGTGCCCATCCT |
| | GGTCGAGCTGGACGGCGACGTAAACGGCCACAAGTTCAGCGTGTC |
| | CGGCGAGGGCGAGGGCGATGCCACCTACGGCAAGCTGACCCTGAA |
| | GTTCATCTGCACCACCGGCAAGCTGCCCGTGCCCTGGCCCACCCT |
| | CGTGACCACCCTGACCTACGGCGTGCAGTGCTTCAGCCGCTACCC |
| | CGACCACATGAAGCAGCACGACTTCTTCAAGTCCGCCA |
| SEQ ID NO: | TGCCCGAAGGCTACGTCCAGGAGCGCACCATCTTCTTCAAGGACG |
| 14 | ACGGCAACTACAAGACCCGCGCCGAGGTGAAGTTCGAGGGCGACA |
| pAM29 | CCCTGGTGAACCGCATCGAGCTGAAGGGCATCGACTTCAAGGAGG |
| 4xS1m after | ACGGCAACATCCTGGGGCACAAGCTGGAGTACAACTACAACAGCC |
| polyA 30- | ACAACGTCTATATCATGGCCGACAAGCAGAAGAACGGCATCAAGG |
| mer | TGAACTTCAAGATCCGCCACAACATCGAGGACGGCAGCGTGCAGC |
| | TCGCCGACCACTACCAGCAGAACACCCCCATCGGCGACGGCCCCG |
| | TGCTGCTGCCCGACAACCACTACCTGAGCACCCAGTCCGCCCTGA |
| | GCAAAGACCCCAACGAGAAGCGCGATCACATGGTCCTGCTGGAGT |
| | TCGTGACCGCCGCCGGGATCACTCTCGGCATGGACGAGCTGTACA |
| | AGTAATAAAGCTCGCTTTCTTGCTGTCCAATTTCTATTAAAGGTT |
| | CCTTTGTTCCCTAAGTCCAACTACTAAACTGGGGGATATTATGAA |
| | GGGCCTTGAGCATCTGGATTCTGCCTAATAAAAAACATTTATTTT |
| | CATTGCAGCTCGCTTTCTTGCTGTCCAATTTCTATTAAAGGTTCC |
| | TTTGTTCCCTAAGTCCAACTACTAAACTGGGGGATATTATGAAGG |
| | GCCTTGAGCATCTGGATTCTGCCTAATAAAAAACATTTATTTTCA |
| | TTGCAAAAAAAAAAAAAAAAAAAAAAAAAAAAAAGGATCCGTAGA |
| | AAATGCGGCCGCCGACCAGAATCATGCAAGTGCGTAAGATAGTCG |
| | CGGGTCGGCGGCCGCATCTGCTGGGAAGCTACGATCCGTAGAAAA |
| | TGCGGCCGCCGACCAGAATCATGCAAGTGCGTAAGATAGTCGCGG |
| | GTCGGCGGCCGCATCTGCTGGGTAGCTGTGAACCGTAGAAAATGC |
| | GGCCGCCGACCAGAATCATGCAAGTGCGTAAGATAGTCGCGGGTC |
| | GGCGGCCGCATCTGCTGGGAAGCTACGATCCGTAGAAAATGCGGC |
| | CGCCGACCAGAATCATGCAAGTGCGTAAGATAGTCGCGGGTCGGC |
| | GGCCGCATCTGCTGGGAAGCTTGAAGAGCCGTACGGGCGCGCCTA |
| | GGCGCGATTCCGCTTCCTCGCTCACTGACTCGCTGCGCTCGGTCG |
| | TTCGGCTGCGGCGAGCGGTATCAGCTCACTCAAAGGCGGTAATAC |
| | GGTTATCCACAGAATCAGGGGATAACGCAGGAAAGAACATGTGAG |
| | CAAAAGGCCAGCAAAAGGCCAGGAACCGTAAAAAGGCCGCGTTGC |
| | TGGCGTTTTTCCATAGGCTCCGCCCCCCTGACGAGCATCACAAAA |
| | ATCGACGCTCAAGTCAGAGGTGGCGAAACCCGACAGGACTATAAA |
| | GATACCAGGCGTTTCCCCCTGGAAGCTCCCTCGTGCGCTCTCCTG |
| | TTCCGACCCTGCCGCTTACCGGATACCTGTCCGCCTTTCTCCCTT |
| | CGGGAAGCGTGGCGCTTTCTCATAGCTCACGCTGTAGGTATCTCA |
| | GTTCGGTGTAGGTCGTTCGCTCCAAGCTGGGCTGTGTGCACGAAC |
| | CCCCCGTTCAGCCCGACCGCTGCGCCTTATCCGGTAACTATCGTC |
| | TTGAGTCCAACCCGGTAAGACACGACTTATCGCCACTGGCAGCAG |
| | CCACTGGTAACAGGATTAGCAGAGCGAGGTATGTAGGCGGTGCTA |
| | CAGAGTTCTTGAAGTGGTGGCCTAACTACGGCTACACTAGAAGAA |
| | CAGTATTTGGTATCTGCGCTCTGCTGAAGCCAGTTACCTTCGGAA |
| | AAAGAGTTGGTAGCTCTTGATCCGGCAAACAAACCACCGCTGGTA |
| | GCGGTGGTTTTTTTGTTTGCAAGCAGCAGATTACGCGCAGAAAAA |
| | AAGGATCTCAAGAAGATCCTTTGATCTTTTCTACGGGGTCTGACG |
| | CTCAGTGGAACGAAAACTCACGTTAAGGGATTTTGGTCATGAGAT |
| | TATCAAAAAGGATCTTCACCTAGATCCTTTTAAATTAAAAATGAA |
| | GTTTTAAATCAATCTAAAGTATATATGAGTAAACTTGGTCTGACA |
| | GTTACCAATGCTTAATCAGTGAGGCACCTATCTCAGCGATCTGTC |
| | TATTTCGTTCATCCATAGTTGCCTGACTCCCCGTCGTGTAGATAA |
| | CTACGATACGGGAGGGCTTACCATCTGGCCCCAGTGCTGCAATGA |
| | TACCGCGAGATCCACGCTCACCGGCTCCAGATTTATCAGCAATAA |
| | ACCAGCCAGCCGGAAGGGCCGAGCGCAGAAGTGGTCCTGCAACTT |
| | TATCCGCCTCCATCCAGTCTATTAATTGTTGCCGGGAAGCTAGAG |
| | TAAGTAGTTCGCCAGTTAATAGTTTGCGCAACGTTGTTGCCATTG |
| | CTACAGGCATCGTGGTGTCACGCTCGTCGTTTGGTATGGCTTCAT |
| | TCAGCTCCGGTTCCCAACGATCAAGGCGAGTTACATGATCCCCCA |
| | TGTTGTGCAAAAAAGCGGTTAGCTCCTTCGGTCCTCCGATCGTTG |
| | TCAGAAGTAAGTTGGCCGCAGTGTTATCACTCATGGTTATGGCAG |
| | CACTGCATAATTCTCTTACTGTCATGCCATCCGTAAGATGCTTTT |
| | CTGTGACTGGTGAGTACTCAACCAAGTCATTCTGAGAATAGTGTA |
| | TGCGGCGACCGAGTTGCTCTTGCCCGGCGTCAATACGGGATAATA |
| | CCGCGCCACATAGCAGAACTTTAAAAGTGCTCATCATTGGAAAAC |
| | GTTCTTCGGGGCGAAAACTCTCAAGGATCTTACCGCTGTTGAGAT |
| | CCAGTTCGATGTAACCCACTCGTGCACCCAACTGATCTTCAGCAT |
| | CTTTTACTTTCACCAGCGTTTCTGGGTGAGCAAAAACAGGAAGGC |
| | AAAATGCCGCAAAAAAGGGAATAAGGGCGACACGGAAATGTTGAA |
| | TACTCATACTCTTCCTTTTTCAATATTATTGAAGCATTTATCAGG |
| | GTTATTGTCTCATGAGCGGATACATATTTGAATGTATTTAGAAAA |
| | ATAAACAAATAGGGGTTCCGCGCACATTTCCCCGAAAAGTGCCAC |
| | CTGACGTCTAAGAAACCATTATTATCATGACATTAACCTATAAAA |
| | ATAGGCGTATCACGAGGCCCTTTCGTC |
| SEQ ID NO: | TAATACGACTCACTATAGGAGAGCGGCCGCTTTTTCAGCAAGATT |
| 15 | AAGCCCAGGGCAGAGCCATCTATTGCTTACATTTGCTTCTGACAC |
| pAM30 | AACTGTGTTCACTAGCAACCTCAAACAGACACCATGGTGAGCAAG |
| 1xS1m after | GGCGAGGAGCTGTTCACCGGGGTGGTGCCCATCCTGGTCGAGCTG |
| 3′UTR | GACGGCGACGTAAACGGCCACAAGTTCAGCGTGTCCGGCGAGGGC |
| | GAGGGCGATGCCACCTACGGCAAGCTGACCCTGAAGTTCATCTGC |
| | ACCACCGGCAAGCTGCCCGTGCCCTGGCCCACCCTCGTGACCACC |
| | CTGACCTACGGCGTGCAGTGCTTCAGCCGCTACCCCGACCACATG |
| | AAGCAGCACGACTTCTTCAAGTCCGCCATGCCCGAAGGCTACGTC |
| | CAGGAGCGCACCATCTTCTTCAAGGACGACGGCAACTACAAGACC |
| | CGCGCCGAGGTGAAGTTCGAGGGCGACACCCTGGTGAACCGCATC |
| | GAGCTGAAGGGCATCGACTTCAAGGAGGACGGCAACATCCTGGGG |
| | CACAAGCTGGAGTACAACTACAACAGCCACAACGTCTATATCATG |
| | GCCGACAAGCAGAAGAACGGCATCAAGGTGAACTTCAAGATCCGC |
| | CACAACATCGAGGACGGCAGCGTGCAGCTCGCCGACCACTACCAG |
| | CAGAACACCCCCATCGGCGACGGCCCCGTGCTGCTGCCCGACAAC |
| | CACTACCTGAGCACCCAGTCCGCCCTGAGCAAAGACCCCAACGAG |
| | AAGCGCGATCACATGGTCCTGCTGGAGTTCGTGACCGCCGCCGGG |
| | ATCACTCTCGGCATGGACGAGCTGTACAAGTAATAAAGCTCGCTT |
| | TCTTGCTGTCCAATTTCTATTAAAGGTTCCTTTGTTCCCTAAGTC |
| | CAACTACTAAACTGGGGGATATTATGAAGGGCCTTGAGCATCTGG |
| | ATTCTGCCTAATAAAAAACATTTATTTTCATTGCAGCTCGCTTTC |
| | TTGCTGTCCAATTTCTATTAAAGGTTCCTTTGTTCCCTAAGTCCA |
| | ACTACTAAACTGGGGGATATTATGAAGGGCCTTGAGCATCTGGAT |
| | TCTGCCTAATAAAAAACATTTATTTTCATTGCGGATCCGTAGAAA |
| | ATGCGGCCGCCGACCAGAATCATGCAAGTGCGTAAGATAGTCGCG |
| | GGTCGGCGGCCGCATCTGCTGGGAAGCTTAAAAAAAAAAAAAAAA |
| | AAAAAAAAAAAAAAGAAGAGCCGTACGGGCGCGCCTAGGCGCGAT |
| | TCCGCTTCCTCGCTCACTGACTCGCTGCGCTCGGTCGTTCGGCTG |
| | CGGCGAGCGGTATCAGCTCACTCAAAGGCGGTAATACGGTTATCC |
| | ACAGAATCAGGGGATAACGCAGGAAAGAACATGTGAGCAAAAGGC |
| | CAGCAAAAGGCCAGGAACCGTAAAAAGGCCGCGTTGCTGGCGTTT |
| | TTCCATAGGCTCCGCCCCCCTGACGAGCATCACAAAAATCGACGC |
| | TCAAGTCAGAGGTGGCGAAACCCGACAGGACTATAAAGATACCAG |
| | GCGTTTCCCCCTGGAAGCTCCCTCGTGCGCTCTCCTGTTCCGACC |
| | CTGCCGCTTACCGGATACCTGTCCGCCTTTCTCCCTTCGGGAAGC |
| | GTGGCGCTTTCTCATAGCTCACGCTGTAGGTATCTCAGTTCGGTG |
| | TAGGTCGTTCGCTCCAAGCTGGGCTGTGTGCACGAACCCCCCGTT |
| | CAGCCCGACCGCTGCGCCTTATCCGGTAACTATCGTCTTGAGTCC |
| | AACCCGGTAAGACACGACTTATCGCCACTGGCAGCAGCCACTGGT |
| | AACAGGATTAGCAGAGCGAGGTATGTAGGCGGTGCTACAGAGTTC |
| | TTGAAGTGGTGGCCTAACTACGGCTACACTAGAAGAACAGTATTT |
| | GGTATCTGCGCTCTGCTGAAGCCAGTTACCTTCGGAAAAAGAGTT |
| | GGTAGCTCTTGATCCGGCAAACAAACCACCGCTGGTAGCGGTGGT |
| | TTTTTTGTTTGCAAGCAGCAGATTACGCGCAGAAAAAAAGGATCT |
| | CAAGAAGATCCTTTGATCTTTTCTACGGGGTCTGACGCTCAGTGG |
| | AACGAAAACTCACGTTAAGGGATTTTGGTCATGAGATTATCAAAA |
| | AGGATCTTCACCTAGATCCTTTTAAATTAAAAATGAAGTTTTAAA |
| | TCAATCTAAAGTATATATGAGTAAACTTGGTCTGACAGTTACCAA |
| | TGCTTAATCAGTGAGGCACCTATCTCAGCGATCTGTCTATTTCGT |
| | TCATCCATAGTTGCCTGACTCCCCGTCGTGTAGATAACTACGATA |
| | CGGGAGGGCTTACCATCTGGCCCCAGTGCTGCAATGATACCGCGA |
| | GATCCACGCTCACCGGCTCCAGATTTATCAGCAATAAACCAGCCA |
| | GCCGGAAGGGCCGAGCGCAGAAGTGGTCCTGCAACTTTATCCGCC |
| | TCCATCCAGTCTATTAATTGTTGCCGGGAAGCTAGAGTAAGTAGT |
| | TCGCCAGTTAATAGTTTGCGCAACGTTGTTGCCATTGCTACAGGC |
| | ATCGTGGTGTCACGCTCGTCGTTTGGTATGGCTTCATTCAGCTCC |
| | GGTTCCCAACGATCAAGGCGAGTTACATGATCCCCCATGTTGTGC |
| | AAAAAAGCGGTTAGCTCCTTCGGTCCTCCGATCGTTGTCAGAAGT |
| | AAGTTGGCCGCAGTGTTATCACTCATGGTTATGGCAGCACTGCAT |
| | AATTCTCTTACTGTCATGCCATCCGTAAGATGCTTTTCTGTGACT |
| | GGTGAGTACTCAACCAAGTCATTCTGAGAATAGTGTATGCGGCGA |
| | CCGAGTTGCTCTTGCCCGGCGTCAATACGGGATAATACCGCGCCA |
| | CATAGCAGAACTTTAAAAGTGCTCATCATTGGAAAACGTTCTTCG |
| | GGGCGAAAACTCTCAAGGATCTTACCGCTGTTGAGATCCAGTTCG |
| | ATGTAACCCACTCGTGCACCCAACTGATCTTCAGCATCTTTTACT |
| | TTCACCAGCGTTTCTGGGTGAGCAAAAACAGGAAGGCAAAATGCC |
| | GCAAAAAAGGGAATAAGGGCGACACGGAAATGTTGAATACTCATA |
| | CTCTTCCTTTTTCAATATTATTGAAGCATTTATCAGGGTTATTGT |
| | CTCATGAGCGGATACATATTTGAATGTATTTAGAAAAATAAACAA |
| | ATAGGGGTTCCGCGCACATTTCCCCGAAAAGTGCCACCTGACGTC |
| | TAAGAAACCATTATTATCATGACATTAACCTATAAAAATAGGCGT |
| | ATCACGAGGCCCTTTCGTC |
| SEQ ID NO: | TAATACGACTCACTATAGGAGAGCGGCCGCTTTTTCAGCAAGATT |
| 16 | AAGCCCAGGGCAGAGCCATCTATTGCTTACATTTGCTTCTGACAC |
| pAM31 | AACTGTGTTCACTAGCAACCTCAAACAGACACCATGGTGAGCAAG |
| 2xS1m after | GGCGAGGAGCTGTTCACCGGGGTGGTGCCCATCCTGGTCGAGCTG |
| 3′ UTR | GACGGCGACGTAAACGGCCACAAGTTCAGCGTGTCCGGCGAGGGC |
| | GAGGGCGATGCCACCTACGGCAAGCTGACCCTGAAGTTCATCTGC |
| | ACCACCGGCAAGCTGCCCGTGCCCTGGCCCACCCTCGTGACCACC |
| | CTGACCTACGGCGTGCAGTGCTTCAGCCGCTACCCCGACCACATG |
| | AAGCAGCACGACTTCTTCAAGTCCGCCATGCCCGAAGGCTACGTC |
| | CAGGAGCGCACCATCTTCTTCAAGGACGACGGCAACTACAAGACC |
| | CGCGCCGAGGTGAAGTTCGAGGGCGACACCCTGGTGAACCGCATC |
| | GAGCTGAAGGGCATCGACTTCAAGGAGGACGGCAACATCCTGGGG |
| | CACAAGCTGGAGTACAACTACAACAGCCACAACGTCTATATCATG |
| | GCCGACAAGCAGAAGAACGGCATCAAGGTGAACTTCAAGATCCGC |
| | CACAACATCGAGGACGGCAGCGTGCAGCTCGCCGACCACTACCAG |
| | CAGAACACCCCCATCGGCGACGGCCCCGTGCTGCTGCCCGACAAC |
| | CACTACCTGAGCACCCAGTCCGCCCTGAGCAAAGACCCCAACGAG |
| | AAGCGCGATCACATGGTCCTGCTGGAGTTCGTGACCGCCGCCGGG |
| | ATCACTCTCGGCATGGACGAGCTGTACAAGTAATAAAGCTCGCTT |
| | TCTTGCTGTCCAATTTCTATTAAAGGTTCCTTTGTTCCCTAAGTC |
| | CAACTACTAAACTGGGGGATATTATGAAGGGCCTTGAGCATCTGG |
| | ATTCTGCCTAATAAAAAACATTTATTTTCATTGCAGCTCGCTTTC |
| | TTGCTGTCCAATTTCTATTAAAGGTTCCTTTGTTCCCTAAGTCCA |
| | ACTACTAAACTGGGGGATATTATGAAGGGCCTTGAGCATCTGGAT |
| | TCTGCCTAATAAAAAACATTTATTTTCATTGCGGATCCGTAGAAA |
| | ATGCGGCCGCCGACCAGAATCATGCAAGTGCGTAAGATAGTCGCG |
| | GGTCGGCGGCCGCATCTGCTGGGAAGCTACGATCCGTAGAAAATG |
| | CGGCCGCCGACCAGAATCATGCAAGTGCGTAAGATAGTCGCGGGT |
| | CGGCGGCCGCATCTGCTGGGAAGCTTAAAAAAAAAAAAAAAAAAA |
| | AAAAAAAAAAAGAAGAGCCGTACGGGCGCGCCTAGGCGCGATTCC |
| | GCTTCCTCGCTCACTGACTCGCTGCGCTCGGTCGTTCGGCTGCGG |
| | CGAGCGGTATCAGCTCACTCAAAGGCGGTAATACGGTTATCCACA |
| | GAATCAGGGGATAACGCAGGAAAGAACATGTGAGCAAAAGGCCAG |
| | CAAAAGGCCAGGAACCGTAAAAAGGCCGCGTTGCTGGCGTTTTTC |
| | CATAGGCTCCGCCCCCCTGACGAGCATCACAAAAATCGACGCTCA |
| | AGTCAGAGGTGGCGAAACCCGACAGGACTATAAAGATACCAGGCG |
| | TTTCCCCCTGGAAGCTCCCTCGTGCGCTCTCCTGTTCCGACCCTG |
| | CCGCTTACCGGATACCTGTCCGCCTTTCTCCCTTCGGGAAGCGTG |
| | GCGCTTTCTCATAGCTCACGCTGTAGGTATCTCAGTTCGGTGTAG |
| | GTCGTTCGCTCCAAGCTGGGCTGTGTGCACGAACCCCCCGTTCAG |
| | CCCGACCGCTGCGCCTTATCCGGTAACTATCGTCTTGAGTCCAAC |
| | CCGGTAAGACACGACTTATCGCCACTGGCAGCAGCCACTGGTAAC |
| | AGGATTAGCAGAGCGAGGTATGTAGGCGGTGCTACAGAGTTCTTG |
| | AAGTGGTGGCCTAACTACGGCTACACTAGAAGAACAGTATTTGGT |
| | ATCTGCGCTCTGCTGAAGCCAGTTACCTTCGGAAAAAGAGTTGGT |
| | AGCTCTTGATCCGGCAAACAAACCACCGCTGGTAGCGGTGGTTTT |
| | TTTGTTTGCAAGCAGCAGATTACGCGCAGAAAAAAAGGATCTCAA |
| | GAAGATCCTTTGATCTTTTCTACGGGGTCTGACGCTCAGTGGAAC |
| | GAAAACTCACGTTAAGGGATTTTGGTCATGAGATTATCAAAAAGG |
| | ATCTTCACCTAGATCCTTTTAAATTAAAAATGAAGTTTTAAATCA |
| | ATCTAAAGTATATATGAGTAAACTTGGTCTGACAGTTACCAATGC |
| | TTAATCAGTGAGGCACCTATCTCAGCGATCTGTCTATTTCGTTCA |
| | TCCATAGTTGCCTGACTCCCCGTCGTGTAGATAACTACGATACGG |
| | GAGGGCTTACCATCTGGCCCCAGTGCTGCAATGATACCGCGAGAT |
| | CCACGCTCACCGGCTCCAGATTTATCAGCAATAAACCAGCCAGCC |
| | GGAAGGGCCGAGCGCAGAAGTGGTCCTGCAACTTTATCCGCCTCC |
| | ATCCAGTCTATTAATTGTTGCCGGGAAGCTAGAGTAAGTAGTTCG |
| | CCAGTTAATAGTTTGCGCAACGTTGTTGCCATTGCTACAGGCATC |
| | GTGGTGTCACGCTCGTCGTTTGGTATGGCTTCATTCAGCTCCGGT |
| | TCCCAACGATCAAGGCGAGTTACATGATCCCCCATGTTGTGCAAA |
| | AAAGCGGTTAGCTCCTTCGGTCCTCCGATCGTTGTCAGAAGTAAG |
| | TTGGCCGCAGTGTTATCACTCATGGTTATGGCAGCACTGCATAAT |
| | TCTCTTACTGTCATGCCATCCGTAAGATGCTTTTCTGTGACTGGT |
| | GAGTACTCAACCAAGTCATTCTGAGAATAGTGTATGCGGCGACCG |
| | AGTTGCTCTTGCCCGGCGTCAATACGGGATAATACCGCGCCACAT |
| | AGCAGAACTTTAAAAGTGCTCATCATTGGAAAACGTTCTTCGGGG |
| | CGAAAACTCTCAAGGATCTTACCGCTGTTGAGATCCAGTTCGATG |
| | TAACCCACTCGTGCACCCAACTGATCTTCAGCATCTTTTACTTTC |
| | ACCAGCGTTTCTGGGTGAGCAAAAACAGGAAGGCAAAATGCCGCA |
| | AAAAAGGGAATAAGGGCGACACGGAAATGTTGAATACTCATACTC |
| | TTCCTTTTTCAATATTATTGAAGCATTTATCAGGGTTATTGTCTC |
| | ATGAGCGGATACATATTTGAATGTATTTAGAAAAATAAACAAATA |
| | GGGGTTCCGCGCACATTTCCCCGAAAAGTGCCACCTGACGTCTAA |
| | GAAACCATTATTATCATGACATTAACCTATAAAAATAGGCGTATC |
| | ACGAGGCCCTTTCGTC |
| SEQ ID NO: | TAATACGACTCACTATAGGAGAGCGGCCGCTTTTTCAGCAAGATT |
| 17 | AAGCCCAGGGCAGAGCCATCTATTGCTTACATTTGCTTCTGACAC |
| pAM32 | AACTGTGTTCACTAGCAACCTCAAACAGACACCATGGTGAGCAAG |
| 3xS1m after | GGCGAGGAGCTGTTCACCGGGGTGGTGCCCATCCTGGTCGAGCTG |
| 3′ UTR | GACGGCGACGTAAACGGCCACAAGTTCAGCGTGTCCGGCGAGGGC |
| | GAGGGCGATGCCACCTACGGCAAGCTGACCCTGAAGTTCATCTGC |
| | ACCACCGGCAAGCTGCCCGTGCCCTGGCCCACCCTCGTGACCACC |
| | CTGACCTACGGCGTGCAGTGCTTCAGCCGCTACCCCGACCACATG |
| | AAGCAGCACGACTTCTTCAAGTCCGCCATGCCCGAAGGCTACGTC |
| | CAGGAGCGCACCATCTTCTTCAAGGACGACGGCAACTACAAGACC |
| | CGCGCCGAGGTGAAGTTCGAGGGCGACACCCTGGTGAACCGCATC |
| | GAGCTGAAGGGCATCGACTTCAAGGAGGACGGCAACATCCTGGGG |
| | CACAAGCTGGAGTACAACTACAACAGCCACAACGTCTATATCATG |
| | GCCGACAAGCAGAAGAACGGCATCAAGGTGAACTTCAAGATCCGC |
| | CACAACATCGAGGACGGCAGCGTGCAGCTCGCCGACCACTACCAG |
| | CAGAACACCCCCATCGGCGACGGCCCCGTGCTGCTGCCCGACAAC |
| | CACTACCTGAGCACCCAGTCCGCCCTGAGCAAAGACCCCAACGAG |
| | AAGCGCGATCACATGGTCCTGCTGGAGTTCGTGACCGCCGCCGGG |
| | ATCACTCTCGGCATGGACGAGCTGTACAAGTAATAAAGCTCGCTT |
| | TCTTGCTGTCCAATTTCTATTAAAGGTTCCTTTGTTCCCTAAGTC |
| | CAACTACTAAACTGGGGGATATTATGAAGGGCCTTGAGCATCTGG |
| | ATTCTGCCTAATAAAAAACATTTATTTTCATTGCAGCTCGCTTTC |
| | TTGCTGTCCAATTTCTATTAAAGGTTCCTTTGTTCCCTAAGTCCA |
| | ACTACTAAACTGGGGGATATTATGAAGGGCCTTGAGCATCTGGAT |
| | TCTGCCTAATAAAAAACATTTATTTTCATTGCGGATCCGTAGAAA |
| | ATGCGGCCGCCGACCAGAATCATGCAAGTGCGTAAGATAGTCGCG |
| | GGTCGGCGGCCGCATCTGCTGGGAAGCTACGATCCGTAGAAAATG |
| | CGGCCGCCGACCAGAATCATGCAAGTGCGTAAGATAGTCGCGGGT |
| | CGGCGGCCGCATCTGCTGGGTAGCTGTGAACCGTAGAAAATGCGG |
| | CCGCCGACCAGAATCATGCAAGTGCGTAAGATAGTCGCGGGTCGG |
| | CGGCCGCATCTGCTGGGAAGCTTAAAAAAAAAAAAAAAAAAAAAA |
| | AAAAAAAAGAAGAGCCGTACGGGCGCGCCTAGGCGCGATTCCGCT |
| | TCCTCGCTCACTGACTCGCTGCGCTCGGTCGTTCGGCTGCGGCGA |
| | GCGGTATCAGCTCACTCAAAGGCGGTAATACGGTTATCCACAGAA |
| | TCAGGGGATAACGCAGGAAAGAACATGTGAGCAAAAGGCCAGCAA |
| | AAGGCCAGGAACCGTAAAAAGGCCGCGTTGCTGGCGTTTTTCCAT |
| | AGGCTCCGCCCCCCTGACGAGCATCACAAAAATCGACGCTCAAGT |
| | CAGAGGTGGCGAAACCCGACAGGACTATAAAGATACCAGGCGTTT |
| | CCCCCTGGAAGCTCCCTCGTGCGCTCTCCTGTTCCGACCCTGCCG |
| | CTTACCGGATACCTGTCCGCCTTTCTCCCTTCGGGAAGCGTGGCG |
| | CTTTCTCATAGCTCACGCTGTAGGTATCTCAGTTCGGTGTAGGTC |
| | GTTCGCTCCAAGCTGGGCTGTGTGCACGAACCCCCCGTTCAGCCC |
| | GACCGCTGCGCCTTATCCGGTAACTATCGTCTTGAGTCCAACCCG |
| | GTAAGACACGACTTATCGCCACTGGCAGCAGCCACTGGTAACAGG |
| | ATTAGCAGAGCGAGGTATGTAGGCGGTGCTACAGAGTTCTTGAAG |
| | TGGTGGCCTAACTACGGCTACACTAGAAGAACAGTATTTGGTATC |
| | TGCGCTCTGCTGAAGCCAGTTACCTTCGGAAAAAGAGTTGGTAGC |
| | TCTTGATCCGGCAAACAAACCACCGCTGGTAGCGGTGGTTTTTTT |
| | GTTTGCAAGCAGCAGATTACGCGCAGAAAAAAAGGATCTCAAGAA |
| | GATCCTTTGATCTTTTCTACGGGGTCTGACGCTCAGTGGAACGAA |
| | AACTCACGTTAAGGGATTTTGGTCATGAGATTATCAAAAAGGATC |
| | TTCACCTAGATCCTTTTAAATTAAAAATGAAGTTTTAAATCAATC |
| | TAAAGTATATATGAGTAAACTTGGTCTGACAGTTACCAATGCTTA |
| | ATCAGTGAGGCACCTATCTCAGCGATCTGTCTATTTCGTTCATCC |
| | ATAGTTGCCTGACTCCCCGTCGTGTAGATAACTACGATACGGGAG |
| | GGCTTACCATCTGGCCCCAGTGCTGCAATGATACCGCGAGATCCA |
| | CGCTCACCGGCTCCAGATTTATCAGCAATAAACCAGCCAGCCGGA |
| | AGGGCCGAGCGCAGAAGTGGTCCTGCAACTTTATCCGCCTCCATC |
| | CAGTCTATTAATTGTTGCCGGGAAGCTAGAGTAAGTAGTTCGCCA |
| | GTTAATAGTTTGCGCAACGTTGTTGCCATTGCTACAGGCATCGTG |
| | GTGTCACGCTCGTCGTTTGGTATGGCTTCATTCAGCTCCGGTTCC |
| | CAACGATCAAGGCGAGTTACATGATCCCCCATGTTGTGCAAAAAA |
| | GCGGTTAGCTCCTTCGGTCCTCCGATCGTTGTCAGAAGTAAGTTG |
| | GCCGCAGTGTTATCACTCATGGTTATGGCAGCACTGCATAATTCT |
| | CTTACTGTCATGCCATCCGTAAGATGCTTTTCTGTGACTGGTGAG |
| | TACTCAACCAAGTCATTCTGAGAATAGTGTATGCGGCGACCGAGT |
| | TGCTCTTGCCCGGCGTCAATACGGGATAATACCGCGCCACATAGC |
| | AGAACTTTAAAAGTGCTCATCATTGGAAAACGTTCTTCGGGGCGA |
| | AAACTCTCAAGGATCTTACCGCTGTTGAGATCCAGTTCGATGTAA |
| | CCCACTCGTGCACCCAACTGATCTTCAGCATCTTTTACTTTCACC |
| | AGCGTTTCTGGGTGAGCAAAAACAGGAAGGCAAAATGCCGCAAAA |
| | AAGGGAATAAGGGCGACACGGAAATGTTGAATACTCATACTCTTC |
| | CTTTTTCAATATTATTGAAGCATTTATCAGGGTTATTGTCTCATG |
| | AGCGGATACATATTTGAATGTATTTAGAAAAATAAACAAATAGGG |
| | GTTCCGCGCACATTTCCCCGAAAAGTGCCACCTGACGTCTAAGAA |
| | ACCATTATTATCATGACATTAACCTATAAAAATAGGCGTATCACG |
| | AGGCCCTTTCGTC |
| SEQ ID NO: | TAATACGACTCACTATAGGAGAGCGGCCGCTTTTTCAGCAAGATT |
| 18 | AAGCCCAGGGCAGAGCCATCTATTGCTTACATTTGCTTCTGACAC |
| pAM33 | AACTGTGTTCACTAGCAACCTCAAACAGACACCATGGTGAGCAAG |
| 5xS1m after | GGCGAGGAGCTGTTCACCGGGGTGGTGCCCATCCTGGTCGAGCTG |
| 3′ UTR | GACGGCGACGTAAACGGCCACAAGTTCAGCGTGTCCGGCGAGGGC |
| | GAGGGCGATGCCACCTACGGCAAGCTGACCCTGAAGTTCATCTGC |
| | ACCACCGGCAAGCTGCCCGTGCCCTGGCCCACCCTCGTGACCACC |
| | CTGACCTACGGCGTGCAGTGCTTCAGCCGCTACCCCGACCACATG |
| | AAGCAGCACGACTTCTTCAAGTCCGCCATGCCCGAAGGCTACGTC |
| | CAGGAGCGCACCATCTTCTTCAAGGACGACGGCAACTACAAGACC |
| | CGCGCCGAGGTGAAGTTCGAGGGCGACACCCTGGTGAACCGCATC |
| | GAGCTGAAGGGCATCGACTTCAAGGAGGACGGCAACATCCTGGGG |
| | CACAAGCTGGAGTACAACTACAACAGCCACAACGTCTATATCATG |
| | GCCGACAAGCAGAAGAACGGCATCAAGGTGAACTTCAAGATCCGC |
| | CACAACATCGAGGACGGCAGCGTGCAGCTCGCCGACCACTACCAG |
| | CAGAACACCCCCATCGGCGACGGCCCCGTGCTGCTGCCCGACAAC |
| | CACTACCTGAGCACCCAGTCCGCCCTGAGCAAAGACCCCAACGAG |
| | AAGCGCGATCACATGGTCCTGCTGGAGTTCGTGACCGCCGCCGGG |
| | ATCACTCTCGGCATGGACGAGCTGTACAAGTAATAAAGCTCGCTT |
| | TCTTGCTGTCCAATTTCTATTAAAGGTTCCTTTGTTCCCTAAGTC |
| | CAACTACTAAACTGGGGGATATTATGAAGGGCCTTGAGCATCTGG |
| | ATTCTGCCTAATAAAAAACATTTATTTTCATTGCAGCTCGCTTTC |
| | TTGCTGTCCAATTTCTATTAAAGGTTCCTTTGTTCCCTAAGTCCA |
| | ACTACTAAACTGGGGGATATTATGAAGGGCCTTGAGCATCTGGAT |
| | TCTGCCTAATAAAAAACATTTATTTTCATTGCGGATCCGTAGAAA |
| | ATGCGGCCGCCGACCAGAATCATGCAAGTGCGTAAGATAGTCGCG |
| | GGTCGGCGGCCGCATCTGCTGGGAAGCTACGATCCGTAGAAAATG |
| | CGGCCGCCGACCAGAATCATGCAAGTGCGTAAGATAGTCGCGGGT |
| | CGGCGGCCGCATCTGCTGGGTAGCTGTGAACCGTAGAAAATGCGG |
| | CCGCCGACCAGAATCATGCAAGTGCGTAAGATAGTCGCGGGTCGG |
| | CGGCCGCATCTGCTGGGAAGCTACGATCCGTAGAAAATGCGGCCG |
| | CCGACCAGAATCATGCAAGTGCGTAAGATAGTCGCGGGTCGGCGG |
| | CCGCATCTGCTGGGTAGCTGTGAACCGTAGAAAATGCGGCCGCCG |
| | ACCAGAATCATGCAAGTGCGTAAGATAGTCGCGGGTCGGCGGCCG |
| | CATCTGCTGGGAAGCTTAAAAAAAAAAAAAAAAAAAAAAAAAAAA |
| | AAGAAGAGCCGTACGGGCGCGCCTAGGCGCGATTCCGCTTCCTCG |
| | CTCACTGACTCGCTGCGCTCGGTCGTTCGGCTGCGGCGAGCGGTA |
| | TCAGCTCACTCAAAGGCGGTAATACGGTTATCCACAGAATCAGGG |
| | GATAACGCAGGAAAGAACATGTGAGCAAAAGGCCAGCAAAAGGCC |
| | AGGAACCGTAAAAAGGCCGCGTTGCTGGCGTTTTTCCATAGGCTC |
| | CGCCCCCCTGACGAGCATCACAAAAATCGACGCTCAAGTCAGAGG |
| | TGGCGAAACCCGACAGGACTATAAAGATACCAGGCGTTTCCCCCT |
| | GGAAGCTCCCTCGTGCGCTCTCCTGTTCCGACCCTGCCGCTTACC |
| | GGATACCTGTCCGCCTTTCTCCCTTCGGGAAGCGTGGCGCTTTCT |
| | CATAGCTCACGCTGTAGGTATCTCAGTTCGGTGTAGGTCGTTCGC |
| | TCCAAGCTGGGCTGTGTGCACGAACCCCCCGTTCAGCCCGACCGC |
| | TGCGCCTTATCCGGTAACTATCGTCTTGAGTCCAACCCGGTAAGA |
| | CACGACTTATCGCCACTGGCAGCAGCCACTGGTAACAGGATTAGC |
| | AGAGCGAGGTATGTAGGCGGTGCTACAGAGTTCTTGAAGTGGTGG |
| | CCTAACTACGGCTACACTAGAAGAACAGTATTTGGTATCTGCGCT |
| | CTGCTGAAGCCAGTTACCTTCGGAAAAAGAGTTGGTAGCTCTTGA |
| | TCCGGCAAACAAACCACCGCTGGTAGCGGTGGTTTTTTTGTTTGC |
| | AAGCAGCAGATTACGCGCAGAAAAAAAGGATCTCAAGAAGATCCT |
| | TTGATCTTTTCTACGGGGTCTGACGCTCAGTGGAACGAAAACTCA |
| | CGTTAAGGGATTTTGGTCATGAGATTATCAAAAAGGATCTTCACC |
| | TAGATCCTTTTAAATTAAAAATGAAGTTTTAAATCAATCTAAAGT |
| | ATATATGAGTAAACTTGGTCTGACAGTTACCAATGCTTAATCAGT |
| | GAGGCACCTATCTCAGCGATCTGTCTATTTCGTTCATCCATAGTT |
| | GCCTGACTCCCCGTCGTGTAGATAACTACGATACGGGAGGGCTTA |
| | CCATCTGGCCCCAGTGCTGCAATGATACCGCGAGATCCACGCTCA |
| | CCGGCTCCAGATTTATCAGCAATAAACCAGCCAGCCGGAAGGGCC |
| | GAGCGCAGAAGTGGTCCTGCAACTTTATCCGCCTCCATCCAGTCT |
| | ATTAATTGTTGCCGGGAAGCTAGAGTAAGTAGTTCGCCAGTTAAT |
| | AGTTTGCGCAACGTTGTTGCCATTGCTACAGGCATCGTGGTGTCA |
| | CGCTCGTCGTTTGGTATGGCTTCATTCAGCTCCGGTTCCCAACGA |
| | TCAAGGCGAGTTACATGATCCCCCATGTTGTGCAAAAAAGCGGTT |
| | AGCTCCTTCGGTCCTCCGATCGTTGTCAGAAGTAAGTTGGCCGCA |
| | GTGTTATCACTCATGGTTATGGCAGCACTGCATAATTCTCTTACT |
| | GTCATGCCATCCGTAAGATGCTTTTCTGTGACTGGTGAGTACTCA |
| | ACCAAGTCATTCTGAGAATAGTGTATGCGGCGACCGAGTTGCTCT |
| | TGCCCGGCGTCAATACGGGATAATACCGCGCCACATAGCAGAACT |
| | TTAAAAGTGCTCATCATTGGAAAACGTTCTTCGGGGCGAAAACTC |
| | TCAAGGATCTTACCGCTGTTGAGATCCAGTTCGATGTAACCCACT |
| | CGTGCACCCAACTGATCTTCAGCATCTTTTACTTTCACCAGCGTT |
| | TCTGGGTGAGCAAAAACAGGAAGGCAAAATGCCGCAAAAAAGGGA |
| | ATAAGGGCGACACGGAAATGTTGAATACTCATACTCTTCCTTTTT |
| | CAATATTATTGAAGCATTTATCAGGGTTATTGTCTCATGAGCGGA |
| | TACATATTTGAATGTATTTAGAAAAATAAACAAATAGGGGTTCCG |
| | CGCACATTTCCCCGAAAAGTGCCACCTGACGTCTAAGAAACCATT |
| | ATTATCATGACATTAACCTATAAAAATAGGCGTATCACGAGGCCC |
| | TTTCGTC |
| SEQ ID NO: | TAATACGACTCACTATAGGAGAGCGGCCGCTTTTTCAGCAAGATT |
| 19 | AAGCCCAGGGCAGAGCCATCTATTGCTTACATTTGCTTCTGACAC |
| pAM34 | AACTGTGTTCACTAGCAACCTCAAACAGACACCATGGTGAGCAAG |
| 6xS1m after | GGCGAGGAGCTGTTCACCGGGGTGGTGCCCATCCTGGTCGAGCTG |
| 3′UTR | GACGGCGACGTAAACGGCCACAAGTTCAGCGTGTCCGGCGAGGGC |
| | GAGGGCGATGCCACCTACGGCAAGCTGACCCTGAAGTTCATCTGC |
| | ACCACCGGCAAGCTGCCCGTGCCCTGGCCCACCCTCGTGACCACC |
| | CTGACCTACGGCGTGCAGTGCTTCAGCCGCTACCCCGACCACATG |
| | AAGCAGCACGACTTCTTCAAGTCCGCCATGCCCGAAGGCTACGTC |
| | CAGGAGCGCACCATCTTCTTCAAGGACGACGGCAACTACAAGACC |
| | CGCGCCGAGGTGAAGTTCGAGGGCGACACCCTGGTGAACCGCATC |
| | GAGCTGAAGGGCATCGACTTCAAGGAGGACGGCAACATCCTGGGG |
| | CACAAGCTGGAGTACAACTACAACAGCCACAACGTCTATATCATG |
| | GCCGACAAGCAGAAGAACGGCATCAAGGTGAACTTCAAGATCCGC |
| | CACAACATCGAGGACGGCAGCGTGCAGCTCGCCGACCACTACCAG |
| | CAGAACACCCCCATCGGCGACGGCCCCGTGCTGCTGCCCGACAAC |
| | CACTACCTGAGCACCCAGTCCGCCCTGAGCAAAGACCCCAACGAG |
| | AAGCGCGATCACATGGTCCTGCTGGAGTTCGTGACCGCCGCCGGG |
| | ATCACTCTCGGCATGGACGAGCTGTACAAGTAATAAAGCTCGCTT |
| | TCTTGCTGTCCAATTTCTATTAAAGGTTCCTTTGTTCCCTAAGTC |
| | CAACTACTAAACTGGGGGATATTATGAAGGGCCTTGAGCATCTGG |
| | ATTCTGCCTAATAAAAAACATTTATTTTCATTGCAGCTCGCTTTC |
| | TTGCTGTCCAATTTCTATTAAAGGTTCCTTTGTTCCCTAAGTCCA |
| | ACTACTAAACTGGGGGATATTATGAAGGGCCTTGAGCATCTGGAT |
| | TCTGCCTAATAAAAAACATTTATTTTCATTGCGGATCCGTAGAAA |
| | ATGCGGCCGCCGACCAGAATCATGCAAGTGCGTAAGATAGTCGCG |
| | GGTCGGCGGCCGCATCTGCTGGGAAGCTACGATCCGTAGAAAATG |
| | CGGCCGCCGACCAGAATCATGCAAGTGCGTAAGATAGTCGCGGGT |
| | CGGCGGCCGCATCTGCTGGGTAGCTGTGAACCGTAGAAAATGCGG |
| | CCGCCGACCAGAATCATGCAAGTGCGTAAGATAGTCGCGGGTCGG |
| | CGGCCGCATCTGCTGGGAAGCTACGATCCGTAGAAAATGCGGCCG |
| | CCGACCAGAATCATGCAAGTGCGTAAGATAGTCGCGGGTCGGCGG |
| | CCGCATCTGCTGGGTAGCTGTGAACCGTAGAAAATGCGGCCGCCG |
| | ACCAGAATCATGCAAGTGCGTAAGATAGTCGCGGGTCGGCGGCCG |
| | CATCTGCTGGGAAGCTACGATCCGTAGAAAATGCGGCCGCCGACC |
| | AGAATCATGCAAGTGCGTAAGATAGTCGCGGGTCGGCGGCCGCAT |
| | CTGCTGGGAAGCTTAAAAAAAAAAAAAAAAAAAAAAAAAAAAAAG |
| | AAGAGCCGTACGGGCGCGCCTAGGCGCGATTCCGCTTCCTCGCTC |
| | ACTGACTCGCTGCGCTCGGTCGTTCGGCTGCGGCGAGCGGTATCA |
| | GCTCACTCAAAGGCGGTAATACGGTTATCCACAGAATCAGGGGAT |
| | AACGCAGGAAAGAACATGTGAGCAAAAGGCCAGCAAAAGGCCAGG |
| | AACCGTAAAAAGGCCGCGTTGCTGGCGTTTTTCCATAGGCTCCGC |
| | CCCCCTGACGAGCATCACAAAAATCGACGCTCAAGTCAGAGGTGG |
| | CGAAACCCGACAGGACTATAAAGATACCAGGCGTTTCCCCCTGGA |
| | AGCTCCCTCGTGCGCTCTCCTGTTCCGACCCTGCCGCTTACCGGA |
| | TACCTGTCCGCCTTTCTCCCTTCGGGAAGCGTGGCGCTTTCTCAT |
| | AGCTCACGCTGTAGGTATCTCAGTTCGGTGTAGGTCGTTCGCTCC |
| | AAGCTGGGCTGTGTGCACGAACCCCCCGTTCAGCCCGACCGCTGC |
| | GCCTTATCCGGTAACTATCGTCTTGAGTCCAACCCGGTAAGACAC |
| | GACTTATCGCCACTGGCAGCAGCCACTGGTAACAGGATTAGCAGA |
| | GCGAGGTATGTAGGCGGTGCTACAGAGTTCTTGAAGTGGTGGCCT |
| | AACTACGGCTACACTAGAAGAACAGTATTTGGTATCTGCGCTCTG |
| | CTGAAGCCAGTTACCTTCGGAAAAAGAGTTGGTAGCTCTTGATCC |
| | GGCAAACAAACCACCGCTGGTAGCGGTGGTTTTTTTGTTTGCAAG |
| | CAGCAGATTACGCGCAGAAAAAAAGGATCTCAAGAAGATCCTTTG |
| | ATCTTTTCTACGGGGTCTGACGCTCAGTGGAACGAAAACTCACGT |
| | TAAGGGATTTTGGTCATGAGATTATCAAAAAGGATCTTCACCTAG |
| | ATCCTTTTAAATTAAAAATGAAGTTTTAAATCAATCTAAAGTATA |
| | TATGAGTAAACTTGGTCTGACAGTTACCAATGCTTAATCAGTGAG |
| | GCACCTATCTCAGCGATCTGTCTATTTCGTTCATCCATAGTTGCC |
| | TGACTCCCCGTCGTGTAGATAACTACGATACGGGAGGGCTTACCA |
| | TCTGGCCCCAGTGCTGCAATGATACCGCGAGATCCACGCTCACCG |
| | GCTCCAGATTTATCAGCAATAAACCAGCCAGCCGGAAGGGCCGAG |
| | CGCAGAAGTGGTCCTGCAACTTTATCCGCCTCCATCCAGTCTATT |
| | AATTGTTGCCGGGAAGCTAGAGTAAGTAGTTCGCCAGTTAATAGT |
| | TTGCGCAACGTTGTTGCCATTGCTACAGGCATCGTGGTGTCACGC |
| | TCGTCGTTTGGTATGGCTTCATTCAGCTCCGGTTCCCAACGATCA |
| | AGGCGAGTTACATGATCCCCCATGTTGTGCAAAAAAGCGGTTAGC |
| | TCCTTCGGTCCTCCGATCGTTGTCAGAAGTAAGTTGGCCGCAGTG |
| | TTATCACTCATGGTTATGGCAGCACTGCATAATTCTCTTACTGTC |
| | ATGCCATCCGTAAGATGCTTTTCTGTGACTGGTGAGTACTCAACC |
| | AAGTCATTCTGAGAATAGTGTATGCGGCGACCGAGTTGCTCTTGC |
| | CCGGCGTCAATACGGGATAATACCGCGCCACATAGCAGAACTTTA |
| | AAAGTGCTCATCATTGGAAAACGTTCTTCGGGGCGAAAACTCTCA |
| | AGGATCTTACCGCTGTTGAGATCCAGTTCGATGTAACCCACTCGT |
| | GCACCCAACTGATCTTCAGCATCTTTTACTTTCACCAGCGTTTCT |
| | GGGTGAGCAAAAACAGGAAGGCAAAATGCCGCAAAAAAGGGAATA |
| | AGGGCGACACGGAAATGTTGAATACTCATACTCTTCCTTTTTCAA |
| | TATTATTGAAGCATTTATCAGGGTTATTGTCTCATGAGCGGATAC |
| | ATATTTGAATGTATTTAGAAAAATAAACAAATAGGGGTTCCGCGC |
| | ACATTTCCCCGAAAAGTGCCACCTGACGTCTAAGAAACCATTATT |
| | ATCATGACATTAACCTATAAAAATAGGCGTATCACGAGGCCCTTT |
| | CGTC |
| SEQ ID NO: | TAATACGACTCACTATAGGAGAGCGGCCGCTTTTTCAGCAAGATT |
| 20 | AAGCCCAGGGCAGAGCCATCTATTGCTTACATTTGCTTCTGACAC |
| pAM37 | AACTGTGTTCACTAGCAACCTCAAACAGACACCATGGTGAGCAAG |
| tRNA | GGCGAGGAGCTGTTCACCGGGGTGGTGCCCATCCTGGTCGAGCTG |
| scaffold | GACGGCGACGTAAACGGCCACAAGTTCAGCGTGTCCGGCGAGGGC |
| stabilized | GAGGGCGATGCCACCTACGGCAAGCTGACCCTGAAGTTCATCTGC |
| S1m after | ACCACCGGCAAGCTGCCCGTGCCCTGGCCCACCCTCGTGACCACC |
| 3′UTR | CTGACCTACGGCGTGCAGTGCTTCAGCCGCTACCCCGACCACATG |
| | AAGCAGCACGACTTCTTCAAGTCCGCCATGCCCGAAGGCTACGTC |
| | CAGGAGCGCACCATCTTCTTCAAGGACGACGGCAACTACAAGACC |
| | CGCGCCGAGGTGAAGTTCGAGGGCGACACCCTGGTGAACCGCATC |
| | GAGCTGAAGGGCATCGACTTCAAGGAGGACGGCAACATCCTGGGG |
| | CACAAGCTGGAGTACAACTACAACAGCCACAACGTCTATATCATG |
| | GCCGACAAGCAGAAGAACGGCATCAAGGTGAACTTCAAGATCCGC |
| | CACAACATCGAGGACGGCAGCGTGCAGCTCGCCGACCACTACCAG |
| | CAGAACACCCCCATCGGCGACGGCCCCGTGCTGCTGCCCGACAAC |
| | CACTACCTGAGCACCCAGTCCGCCCTGAGCAAAGACCCCAACGAG |
| | AAGCGCGATCACATGGTCCTGCTGGAGTTCGTGACCGCCGCCGGG |
| | ATCACTCTCGGCATGGACGAGCTGTACAAGTAATAAAGCTCGCTT |
| | TCTTGCTGTCCAATTTCTATTAAAGGTTCCTTTGTTCCCTAAGTC |
| | CAACTACTAAACTGGGGGATATTATGAAGGGCCTTGAGCATCTGG |
| | ATTCTGCCTAATAAAAAACATTTATTTTCATTGCAGCTCGCTTTC |
| | TTGCTGTCCAATTTCTATTAAAGGTTCCTTTGTTCCCTAAGTCCA |
| | ACTACTAAACTGGGGGATATTATGAAGGGCCTTGAGCATCTGGAT |
| | TCTGCCTAATAAAAAACATTTATTTTCATTGCGGATCCAAAAAAA |
| | AAAAAAGCCCGGATAGCTCAGTCGGTAGAGCAGCGGCCTATGCGG |
| | CCGCCGACCAGAATCATGCAAGTGCGTAAGATAGTCGCGGGTCGG |
| | CGGCCGCATTCGAGGCCGCGTCCAGGGTTCAAGTCCCTGTTCGGG |
| | CGCCACTGCAGAAAAAAAAAAAAAAGCTTAAAAAAAAAAAAAAAA |
| | AAAAAAAAAAAAAAGAAGAGCCGTACGGGCGCGCCTAGGCGCGAT |
| | TCCGCTTCCTCGCTCACTGACTCGCTGCGCTCGGTCGTTCGGCTG |
| | CGGCGAGCGGTATCAGCTCACTCAAAGGCGGTAATACGGTTATCC |
| | ACAGAATCAGGGGATAACGCAGGAAAGAACATGTGAGCAAAAGGC |
| | CAGCAAAAGGCCAGGAACCGTAAAAAGGCCGCGTTGCTGGCGTTT |
| | TTCCATAGGCTCCGCCCCCCTGACGAGCATCACAAAAATCGACGC |
| | TCAAGTCAGAGGTGGCGAAACCCGACAGGACTATAAAGATACCAG |
| | GCGTTTCCCCCTGGAAGCTCCCTCGTGCGCTCTCCTGTTCCGACC |
| | CTGCCGCTTACCGGATACCTGTCCGCCTTTCTCCCTTCGGGAAGC |
| | GTGGCGCTTTCTCATAGCTCACGCTGTAGGTATCTCAGTTCGGTG |
| | TAGGTCGTTCGCTCCAAGCTGGGCTGTGTGCACGAACCCCCCGTT |
| | CAGCCCGACCGCTGCGCCTTATCCGGTAACTATCGTCTTGAGTCC |
| | AACCCGGTAAGACACGACTTATCGCCACTGGCAGCAGCCACTGGT |
| | AACAGGATTAGCAGAGCGAGGTATGTAGGCGGTGCTACAGAGTTC |
| | TTGAAGTGGTGGCCTAACTACGGCTACACTAGAAGAACAGTATTT |
| | GGTATCTGCGCTCTGCTGAAGCCAGTTACCTTCGGAAAAAGAGTT |
| | GGTAGCTCTTGATCCGGCAAACAAACCACCGCTGGTAGCGGTGGT |
| | TTTTTTGTTTGCAAGCAGCAGATTACGCGCAGAAAAAAAGGATCT |
| | CAAGAAGATCCTTTGATCTTTTCTACGGGGTCTGACGCTCAGTGG |
| | AACGAAAACTCACGTTAAGGGATTTTGGTCATGAGATTATCAAAA |
| | AGGATCTTCACCTAGATCCTTTTAAATTAAAAATGAAGTTTTAAA |
| | TCAATCTAAAGTATATATGAGTAAACTTGGTCTGACAGTTACCAA |
| | TGCTTAATCAGTGAGGCACCTATCTCAGCGATCTGTCTATTTCGT |
| | TCATCCATAGTTGCCTGACTCCCCGTCGTGTAGATAACTACGATA |
| | CGGGAGGGCTTACCATCTGGCCCCAGTGCTGCAATGATACCGCGA |
| | GATCCACGCTCACCGGCTCCAGATTTATCAGCAATAAACCAGCCA |
| | GCCGGAAGGGCCGAGCGCAGAAGTGGTCCTGCAACTTTATCCGCC |
| | TCCATCCAGTCTATTAATTGTTGCCGGGAAGCTAGAGTAAGTAGT |
| | TCGCCAGTTAATAGTTTGCGCAACGTTGTTGCCATTGCTACAGGC |
| | ATCGTGGTGTCACGCTCGTCGTTTGGTATGGCTTCATTCAGCTCC |
| | GGTTCCCAACGATCAAGGCGAGTTACATGATCCCCCATGTTGTGC |
| | AAAAAAGCGGTTAGCTCCTTCGGTCCTCCGATCGTTGTCAGAAGT |
| | AAGTTGGCCGCAGTGTTATCACTCATGGTTATGGCAGCACTGCAT |
| | AATTCTCTTACTGTCATGCCATCCGTAAGATGCTTTTCTGTGACT |
| | GGTGAGTACTCAACCAAGTCATTCTGAGAATAGTGTATGCGGCGA |
| | CCGAGTTGCTCTTGCCCGGCGTCAATACGGGATAATACCGCGCCA |
| | CATAGCAGAACTTTAAAAGTGCTCATCATTGGAAAACGTTCTTCG |
| | GGGCGAAAACTCTCAAGGATCTTACCGCTGTTGAGATCCAGTTCG |
| | ATGTAACCCACTCGTGCACCCAACTGATCTTCAGCATCTTTTACT |
| | TTCACCAGCGTTTCTGGGTGAGCAAAAACAGGAAGGCAAAATGCC |
| | GCAAAAAAGGGAATAAGGGCGACACGGAAATGTTGAATACTCATA |
| | CTCTTCCTTTTTCAATATTATTGAAGCATTTATCAGGGTTATTGT |
| | CTCATGAGCGGATACATATTTGAATGTATTTAGAAAAATAAACAA |
| | ATAGGGGTTCCGCGCACATTTCCCCGAAAAGTGCCACCTGACGTC |
| | TAAGAAACCATTATTATCATGACATTAACCTATAAAAATAGGCGT |
| | ATCACGAGGCCCTTTCGTC |
| SEQ ID NO: | TCGCGCGTTTCGGTGATGACGGTGAAAACCTCTGACACATGCAGC |
| 21 | TCCCGGAGACGGTCACAGCTTGTCTGTAAGCGGATGCCGGGAGCA |
| HA_H3SIN- | GACAAGCCCGTCAGGGCGCGTCAGCGGGTGTTGGCGGGTGTCGGG |
| 16 | GCTGGCTTAACTATGCGGCATCAGAGCAGATTGTACTGAGAGTGC |
| No aptamer | ACCATATGCGGTGTGAAATACCGCACAGATGCGTAAGGAGAAAAT |
| | ACCGCATCAGGCGCCATTCGCCATTCAGGCTGCGCAACTGTTGGG |
| | AAGGGCGATCGGTGCGGGCCTCTTCGCTATTACGCCAGCTGGCGA |
| | AAGGGGGATGTGCTGCAAGGCGATTAAGTTGGGTAACGCCAGGGT |
| | TTTCCCAGTCACGACGTTGTAAAACGACGGCCAGAGAATTCTAGA |
| | ATTTAGGTGACACTATAGGACAGATCGCCTGGAGACGCCATCCAC |
| | GCTGTTTTGACCTCCATAGAAGACACCGGGACCGATCCAGCCTCC |
| | GCGGCCGGGAACGGTGCATTGGAACGCGGATTCCCCGTGCCAAGA |
| | GTGACTCACCGTCCTTGACACGATGAAAACTATTATTGCTCTGTC |
| | TTACATCCTGTGCCTGGTCTTCGCCCAGAAAATCCCCGGAAACGA |
| | CAACTCTACCGCCACCCTGTGTCTGGGCCACCACGCCGTGCCAAA |
| | TGGCACCATCGTGAAGACCATCACAAACGACAGAATCGAGGTGAC |
| | CAATGCCACAGAGCTGGTGCAGAACAGCTCCATCGGCGAGATCTG |
| | CGACAGCCCCCACCAGATCCTGGATGGCGAGAACTGTACACTGAT |
| | CGACGCCCTGCTGGGCGACCCTCAGTGCGATGGCTTCCAGAATAA |
| | GAAGTGGGATCTGTTTGTGGAGAGAAGCAAGGCCTACTCCAACTG |
| | TTACCCCTATGACGTGCCTGATTATGCCTCTCTGAGGAGCCTGGT |
| | GGCCTCTAGCGGCACCCTGGAGTTCAAGAACGAGTCTTTTAATTG |
| | GACCGGCGTGACACAGAATGGCACATCCTCTGCCTGCATCCGCGG |
| | CAGCTCCTCTAGCTTCTTTAGCCGGCTGAACTGGCTGACCCACCT |
| | GAATTACACATATCCTGCCCTGAACGTGACCATGCCAAATAAGGA |
| | GCAGTTCGATAAGCTGTACATCTGGGGAGTGCACCACCCAGGAAC |
| | AGACAAGGATCAGATCTTTCTGTATGCCCAGTCCTCTGGCAGAAT |
| | CACCGTGTCTACAAAGAGGAGCCAGCAGGCCGTGATCCCTAACAT |
| | CGGCTCCCGGCCAAGAATCAGGGACATCCCCTCCCGCATCTCTAT |
| | CTACTGGACCATCGTGAAGCCAGGCGATATCCTGCTGATCAACTC |
| | TACAGGCAATCTGATCGCCCCCCGCGGCTATTTCAAGATCCGGAG |
| | CGGCAAGAGCAGCATCATGCGGTCCGACGCCCCCATCGGCAAGTG |
| | CAAGTCTGAGTGTATCACCCCTAACGGCAGCATCCCAAATGATAA |
| | GCCCTTTCAGAACGTGAATCGCATCACATACGGCGCCTGTCCTAG |
| | ATATGTGAAGCACAGCACCCTGAAGCTGGCCACAGGCATGAGAAA |
| | TGTGCCAGAGAAGCAGACCAGGGGAATCTTCGGAGCAATCGCAGG |
| | CTTTATCGAGAATGGCTGGGAGGGCATGGTGGACGGCTGGTACGG |
| | CTTCCGCCACCAGAACTCCGAGGGAAGGGGACAGGCCGCCGACCT |
| | GAAGTCTACCCAGGCAGCCATCGATCAGATCAACGGCAAGCTGAA |
| | TCGCCTGATCGGCAAGACAAACGAGAAGTTCCACCAGATCGAGAA |
| | GGAGTTTTCCGAGGTGGAGGGAAGGGTGCAGGATCTGGAGAAGTA |
| | CGTGGAGGACACCAAGATCGATCTGTGGAGCTATAATGCCGAGCT |
| | GCTGGTGGCCCTGGAGAACCAGCACACCATCGACCTGACAGATTC |
| | CGAGATGAATAAGCTGTTCGAGAAGACCAAGAAGCAGCTGAGAGA |
| | GAACGCCGAGGACATGGGCAATGGCTGCTTTAAGATCTACCACAA |
| | GTGCGATAACGCCTGTATCGAGAGCATCAGGAACGAGACATACGA |
| | CCACAACGTGTACAGAGATGAGGCCCTGAACAATAGGTTTCAGAT |
| | CAAGGGCGTGGAGCTGAAGTCCGGCTATAAGGACTGGATCCTGTG |
| | GATCTCCTTCGCCATCTCTTGCTTTCTGCTGTGCGTGGCCCTGCT |
| | GGGGTTCATTATGTGGGCTTGTCAGAAAGGAAACATTCGCTGTAA |
| | CATTTGTATCTAACGGGTGGCATCCCTGTGACCCCTCCCCAGTGC |
| | CTCTCCTGGCCCTGGAAGTTGCCACTCCAGTGCCCACCAGCCTTG |
| | TCCTAATAAAATTAAGTTGCATCAAGCTTGGTGTAATCATGGTCA |
| | TAGCTGTTTCCTGTGTGAAATTGTTATCCGCTCACAATTCCACAC |
| | AACATACGAGCCGGAAGCATAAAGTGTAAAGCCTGGGGTGCCTAA |
| | TGAGTGAGCTAACTCACATTAATTGCGTTGCGCTCACTGCCCGCT |
| | TTCCAGTCGGGAAACCTGTCGTGCCAGCTGCATTAATGAATCGGC |
| | CAACGCGCGGGGAGAGGCGGTTTGCGTATTGGGCGCTCTTCCGCT |
| | TCCTCGCTCACTGACTCGCTGCGCTCGGTCGTTCGGCTGCGGCGA |
| | GCGGTATCAGCTCACTCAAAGGCGGTAATACGGTTATCCACAGAA |
| | TCAGGGGATAACGCAGGAAAGAACATGTGAGCAAAAGGCCAGCAA |
| | AAGGCCAGGAACCGTAAAAAGGCCGCGTTGCTGGCGTTTTTCCAT |
| | AGGCTCCGCCCCCCTGACGAGCATCACAAAAATCGACGCTCAAGT |
| | CAGAGGTGGCGAAACCCGACAGGACTATAAAGATACCAGGCGTTT |
| | CCCCCTGGAAGCTCCCTCGTGCGCTCTCCTGTTCCGACCCTGCCG |
| | CTTACCGGATACCTGTCCGCCTTTCTCCCTTCGGGAAGCGTGGCG |
| | CTTTCTCATAGCTCACGCTGTAGGTATCTCAGTTCGGTGTAGGTC |
| | GTTCGCTCCAAGCTGGGCTGTGTGCACGAACCCCCCGTTCAGCCC |
| | GACCGCTGCGCCTTATCCGGTAACTATCGTCTTGAGTCCAACCCG |
| | GTAAGACACGACTTATCGCCACTGGCAGCAGCCACTGGTAACAGG |
| | ATTAGCAGAGCGAGGTATGTAGGCGGTGCTACAGAGTTCTTGAAG |
| | TGGTGGCCTAACTACGGCTACACTAGAAGAACAGTATTTGGTATC |
| | TGCGCTCTGCTGAAGCCAGTTACCTTCGGAAAAAGAGTTGGTAGC |
| | TCTTGATCCGGCAAACAAACCACCGCTGGTAGCGGTGGTTTTTTT |
| | GTTTGCAAGCAGCAGATTACGCGCAGAAAAAAAGGATCTCAAGAA |
| | GATCCTTTGATCTTTTCTACGGGGTCTGACGCTCAGTGGAACGAA |
| | AACTCACGTTAAGGGATTTTGGTCATGAGATTATCAAAAAGGATC |
| | TTCACCTAGATCCTTTTAAATTAAAAATGAAGTTTTAAATCAAGC |
| | CCAATCTGAATAATGTTACAACCAATTAACCAATTCTGATTAGAA |
| | AAACTCATCGAGCATCAAATGAAACTGCAATTTATTCATATCAGG |
| | ATTATCAATACCATATTTTTGAAAAAGCCGTTTCTGTAATGAAGG |
| | AGAAAACTCACCGAGGCAGTTCCATAGGATGGCAAGATCCTGGTA |
| | TCGGTCTGCGATTCCGACTCGTCCAACATCAATACAACCTATTAA |
| | TTTCCCCTCGTCAAAAATAAGGTTATCAAGTGAGAAATCACCATG |
| | AGTGACGACTGAATCCGGTGAGAATGGCAAAAGTTTATGCATTTC |
| | TTTCCAGACTTGTTCAACAGGCCAGCCATTACGCTCGTCATCAAA |
| | ATCACTCGCATCAACCAAACCGTTATTCATTCGTGATTGCGCCTG |
| | AGCGAGACGAAATACGCGATCGCTGTTAAAAGGACAATTACAAAC |
| | AGGAATCGAATGCAACCGGCGCAGGAACACTGCCAGCGCATCAAC |
| | AATATTTTCACCTGAATCAGGATATTCTTCTAATACCTGGAATGC |
| | TGTTTTTCCGGGGATCGCAGTGGTGAGTAACCATGCATCATCAGG |
| | AGTACGGATAAAATGCTTGATGGTCGGAAGAGGCATAAATTCCGT |
| | CAGCCAGTTTAGTCTGACCATCTCATCTGTAACATCATTGGCAAC |
| | GCTACCTTTGCCATGTTTCAGAAACAACTCTGGCGCATCGGGCTT |
| | CCCATACAAGCGATAGATTGTCGCACCTGATTGCCCGACATTATC |
| | GCGAGCCCATTTATACCCATATAAATCAGCATCCATGTTGGAATT |
| | TAATCGCGGCCTCGACGTTTCCCGTTGAATATGGCTCATAACACC |
| | CCTTGTATTACTGTTTATGTAAGCAGACAGTTTTATTGTTCATGA |
| | TGATATATTTTTATCTTGTGCAATGTAACATCAGAGATTTTGAGA |
| | CACGGGCCAGAGCTGCA |
| SEQ ID NO: | TCGCGCGTTTCGGTGATGACGGTGAAAACCTCTGACACATGCAGC |
| 22 | TCCCGGAGACGGTCACAGCTTGTCTGTAAGCGGATGCCGGGAGCA |
| pAM111 | GACAAGCCCGTCAGGGCGCGTCAGCGGGTGTTGGCGGGTGTCGGG |
| No aptamer | GCTGGCTTAACTATGCGGCATCAGAGCAGATTGTACTGAGAGTGC |
| | ACCATATGCGGTGTGAAATACCGCACAGATGCGTAAGGAGAAAAT |
| | ACCGCATCAGGCGCCATTCGCCATTCAGGCTGCGCAACTGTTGGG |
| | AAGGGCGATCGGTGCGGGCCTCTTCGCTATTACGCCAGCTGGCGA |
| | AAGGGGGATGTGCTGCAAGGCGATTAAGTTGGGTAACGCCAGGGT |
| | TTTCCCAGTCACGACGTTGTAAAACGACGGCCAGAGAATTCTAGA |
| | ATTTAGGTGACACTATAGGACAGATCGCCTGGAGACGCCATCCAC |
| | GCTGTTTTGACCTCCATAGAAGACACCGGGACCGATCCAGCCTCC |
| | GCGGCCGGGAACGGTGCATTGGAACGCGGATTCCCCGTGCCAAGA |
| | GTGACTCACCGTCCTTGACACGATGAAAACTATTATTGCTCTGTC |
| | TTACATCCTGTGCCTGGTCTTCGCCCAGAAAATCCCCGGAAACGA |
| | CAACTCTACCGCCACCCTGTGTCTGGGCCACCACGCCGTGCCAAA |
| | TGGCACCATCGTGAAGACCATCACAAACGACAGAATCGAGGTGAC |
| | CAATGCCACAGAGCTGGTGCAGAACAGCTCCATCGGCGAGATCTG |
| | CGACAGCCCCCACCAGATCCTGGATGGCGAGAACTGTACACTGAT |
| | CGACGCCCTGCTGGGCGACCCTCAGTGCGATGGCTTCCAGAATAA |
| | GAAGTGGGATCTGTTTGTGGAGAGAAGCAAGGCCTACTCCAACTG |
| | TTACCCCTATGACGTGCCTGATTATGCCTCTCTGAGGAGCCTGGT |
| | GGCCTCTAGCGGCACCCTGGAGTTCAAGAACGAGTCTTTTAATTG |
| | GACCGGCGTGACACAGAATGGCACATCCTCTGCCTGCATCCGCGG |
| | CAGCTCCTCTAGCTTCTTTAGCCGGCTGAACTGGCTGACCCACCT |
| | GAATTACACATATCCTGCCCTGAACGTGACCATGCCAAATAAGGA |
| | GCAGTTCGATAAGCTGTACATCTGGGGAGTGCACCACCCAGGAAC |
| | AGACAAGGATCAGATCTTTCTGTATGCCCAGTCCTCTGGCAGAAT |
| | CACCGTGTCTACAAAGAGGAGCCAGCAGGCCGTGATCCCTAACAT |
| | CGGCTCCCGGCCAAGAATCAGGGACATCCCCTCCCGCATCTCTAT |
| | CTACTGGACCATCGTGAAGCCAGGCGATATCCTGCTGATCAACTC |
| | TACAGGCAATCTGATCGCCCCCCGCGGCTATTTCAAGATCCGGAG |
| | CGGCAAGAGCAGCATCATGCGGTCCGACGCCCCCATCGGCAAGTG |
| | CAAGTCTGAGTGTATCACCCCTAACGGCAGCATCCCAAATGATAA |
| | GCCCTTTCAGAACGTGAATCGCATCACATACGGCGCCTGTCCTAG |
| | ATATGTGAAGCACAGCACCCTGAAGCTGGCCACAGGCATGAGAAA |
| | TGTGCCAGAGAAGCAGACCAGGGGAATCTTCGGAGCAATCGCAGG |
| | CTTTATCGAGAATGGCTGGGAGGGCATGGTGGACGGCTGGTACGG |
| | CTTCCGCCACCAGAACTCCGAGGGAAGGGGACAGGCCGCCGACCT |
| | GAAGTCTACCCAGGCAGCCATCGATCAGATCAACGGCAAGCTGAA |
| | TCGCCTGATCGGCAAGACAAACGAGAAGTTCCACCAGATCGAGAA |
| | GGAGTTTTCCGAGGTGGAGGGAAGGGTGCAGGATCTGGAGAAGTA |
| | CGTGGAGGACACCAAGATCGATCTGTGGAGCTATAATGCCGAGCT |
| | GCTGGTGGCCCTGGAGAACCAGCACACCATCGACCTGACAGATTC |
| | CGAGATGAATAAGCTGTTCGAGAAGACCAAGAAGCAGCTGAGAGA |
| | GAACGCCGAGGACATGGGCAATGGCTGCTTTAAGATCTACCACAA |
| | GTGCGATAACGCCTGTATCGAGAGCATCAGGAACGAGACATACGA |
| | CCACAACGTGTACAGAGATGAGGCCCTGAACAATAGGTTTCAGAT |
| | CAAGGGCGTGGAGCTGAAGTCCGGCTATAAGGACTGGATCCTGTG |
| | GATCTCCTTCGCCATCTCTTGCTTTCTGCTGTGCGTGGCCCTGCT |
| | GGGGTTCATTATGTGGGCTTGTCAGAAAGGAAACATTCGCTGTAA |
| | CATTTGTATCTAACGGGTGGCATCCCTGTGACCCCTCCCCAGTGC |
| | CTCTCCTGGCCCTGGAAGTTGCCACTCCAGTGCCCACCAGCCTTG |
| | TCCTAATAAAATTAAGTTGCATCAAAAAAAAAAAAAAAAAAAAAA |
| | AAAAAAAAAAGCTTGGTGTAATCATGGTCATAGCTGTTTCCTGTG |
| | TGAAATTGTTATCCGCTCACAATTCCACACAACATACGAGCCGGA |
| | AGCATAAAGTGTAAAGCCTGGGGTGCCTAATGAGTGAGCTAACTC |
| | ACATTAATTGCGTTGCGCTCACTGCCCGCTTTCCAGTCGGGAAAC |
| | CTGTCGTGCCAGCTGCATTAATGAATCGGCCAACGCGCGGGGAGA |
| | GGCGGTTTGCGTATTGGGCGCTCTTCCGCTTCCTCGCTCACTGAC |
| | TCGCTGCGCTCGGTCGTTCGGCTGCGGCGAGCGGTATCAGCTCAC |
| | TCAAAGGCGGTAATACGGTTATCCACAGAATCAGGGGATAACGCA |
| | GGAAAGAACATGTGAGCAAAAGGCCAGCAAAAGGCCAGGAACCGT |
| | AAAAAGGCCGCGTTGCTGGCGTTTTTCCATAGGCTCCGCCCCCCT |
| | GACGAGCATCACAAAAATCGACGCTCAAGTCAGAGGTGGCGAAAC |
| | CCGACAGGACTATAAAGATACCAGGCGTTTCCCCCTGGAAGCTCC |
| | CTCGTGCGCTCTCCTGTTCCGACCCTGCCGCTTACCGGATACCTG |
| | TCCGCCTTTCTCCCTTCGGGAAGCGTGGCGCTTTCTCATAGCTCA |
| | CGCTGTAGGTATCTCAGTTCGGTGTAGGTCGTTCGCTCCAAGCTG |
| | GGCTGTGTGCACGAACCCCCCGTTCAGCCCGACCGCTGCGCCTTA |
| | TCCGGTAACTATCGTCTTGAGTCCAACCCGGTAAGACACGACTTA |
| | TCGCCACTGGCAGCAGCCACTGGTAACAGGATTAGCAGAGCGAGG |
| | TATGTAGGCGGTGCTACAGAGTTCTTGAAGTGGTGGCCTAACTAC |
| | GGCTACACTAGAAGAACAGTATTTGGTATCTGCGCTCTGCTGAAG |
| | CCAGTTACCTTCGGAAAAAGAGTTGGTAGCTCTTGATCCGGCAAA |
| | CAAACCACCGCTGGTAGCGGTGGTTTTTTTGTTTGCAAGCAGCAG |
| | ATTACGCGCAGAAAAAAAGGATCTCAAGAAGATCCTTTGATCTTT |
| | TCTACGGGGTCTGACGCTCAGTGGAACGAAAACTCACGTTAAGGG |
| | ATTTTGGTCATGAGATTATCAAAAAGGATCTTCACCTAGATCCTT |
| | TTAAATTAAAAATGAAGTTTTAAATCAAGCCCAATCTGAATAATG |
| | TTACAACCAATTAACCAATTCTGATTAGAAAAACTCATCGAGCAT |
| | CAAATGAAACTGCAATTTATTCATATCAGGATTATCAATACCATA |
| | TTTTTGAAAAAGCCGTTTCTGTAATGAAGGAGAAAACTCACCGAG |
| | GCAGTTCCATAGGATGGCAAGATCCTGGTATCGGTCTGCGATTCC |
| | GACTCGTCCAACATCAATACAACCTATTAATTTCCCCTCGTCAAA |
| | AATAAGGTTATCAAGTGAGAAATCACCATGAGTGACGACTGAATC |
| | CGGTGAGAATGGCAAAAGTTTATGCATTTCTTTCCAGACTTGTTC |
| | AACAGGCCAGCCATTACGCTCGTCATCAAAATCACTCGCATCAAC |
| | CAAACCGTTATTCATTCGTGATTGCGCCTGAGCGAGACGAAATAC |
| | GCGATCGCTGTTAAAAGGACAATTACAAACAGGAATCGAATGCAA |
| | CCGGCGCAGGAACACTGCCAGCGCATCAACAATATTTTCACCTGA |
| | ATCAGGATATTCTTCTAATACCTGGAATGCTGTTTTTCCGGGGAT |
| | CGCAGTGGTGAGTAACCATGCATCATCAGGAGTACGGATAAAATG |
| | CTTGATGGTCGGAAGAGGCATAAATTCCGTCAGCCAGTTTAGTCT |
| | GACCATCTCATCTGTAACATCATTGGCAACGCTACCTTTGCCATG |
| | TTTCAGAAACAACTCTGGCGCATCGGGCTTCCCATACAAGCGATA |
| | GATTGTCGCACCTGATTGCCCGACATTATCGCGAGCCCATTTATA |
| | CCCATATAAATCAGCATCCATGTTGGAATTTAATCGCGGCCTCGA |
| | CGTTTCCCGTTGAATATGGCTCATAACACCCCTTGTATTACTGTT |
| | TATGTAAGCAGACAGTTTTATTGTTCATGATGATATATTTTTATC |
| | TTGTGCAATGTAACATCAGAGATTTTGAGACACGGGCCAGAGCTG |
| | CA |
| SEQ ID NO: | TCGCGCGTTTCGGTGATGACGGTGAAAACCTCTGACACATGCAGC |
| 23 | TCCCGGAGACGGTCACAGCTTGTCTGTAAGCGGATGCCGGGAGCA |
| pAM112 | GACAAGCCCGTCAGGGCGCGTCAGCGGGTGTTGGCGGGTGTCGGG |
| 2xS1m after | GCTGGCTTAACTATGCGGCATCAGAGCAGATTGTACTGAGAGTGC |
| 3′ UTR | ACCATATGCGGTGTGAAATACCGCACAGATGCGTAAGGAGAAAAT |
| | ACCGCATCAGGCGCCATTCGCCATTCAGGCTGCGCAACTGTTGGG |
| | AAGGGCGATCGGTGCGGGCCTCTTCGCTATTACGCCAGCTGGCGA |
| | AAGGGGGATGTGCTGCAAGGCGATTAAGTTGGGTAACGCCAGGGT |
| | TTTCCCAGTCACGACGTTGTAAAACGACGGCCAGAGAATTCTAGA |
| | ATTTAGGTGACACTATAGGACAGATCGCCTGGAGACGCCATCCAC |
| | GCTGTTTTGACCTCCATAGAAGACACCGGGACCGATCCAGCCTCC |
| | GCGGCCGGGAACGGTGCATTGGAACGCGGATTCCCCGTGCCAAGA |
| | GTGACTCACCGTCCTTGACACGATGAAAACTATTATTGCTCTGTC |
| | TTACATCCTGTGCCTGGTCTTCGCCCAGAAAATCCCCGGAAACGA |
| | CAACTCTACCGCCACCCTGTGTCTGGGCCACCACGCCGTGCCAAA |
| | TGGCACCATCGTGAAGACCATCACAAACGACAGAATCGAGGTGAC |
| | CAATGCCACAGAGCTGGTGCAGAACAGCTCCATCGGCGAGATCTG |
| | CGACAGCCCCCACCAGATCCTGGATGGCGAGAACTGTACACTGAT |
| | CGACGCCCTGCTGGGCGACCCTCAGTGCGATGGCTTCCAGAATAA |
| | GAAGTGGGATCTGTTTGTGGAGAGAAGCAAGGCCTACTCCAACTG |
| | TTACCCCTATGACGTGCCTGATTATGCCTCTCTGAGGAGCCTGGT |
| | GGCCTCTAGCGGCACCCTGGAGTTCAAGAACGAGTCTTTTAATTG |
| | GACCGGCGTGACACAGAATGGCACATCCTCTGCCTGCATCCGCGG |
| | CAGCTCCTCTAGCTTCTTTAGCCGGCTGAACTGGCTGACCCACCT |
| | GAATTACACATATCCTGCCCTGAACGTGACCATGCCAAATAAGGA |
| | GCAGTTCGATAAGCTGTACATCTGGGGAGTGCACCACCCAGGAAC |
| | AGACAAGGATCAGATCTTTCTGTATGCCCAGTCCTCTGGCAGAAT |
| | CACCGTGTCTACAAAGAGGAGCCAGCAGGCCGTGATCCCTAACAT |
| | CGGCTCCCGGCCAAGAATCAGGGACATCCCCTCCCGCATCTCTAT |
| | CTACTGGACCATCGTGAAGCCAGGCGATATCCTGCTGATCAACTC |
| | TACAGGCAATCTGATCGCCCCCCGCGGCTATTTCAAGATCCGGAG |
| | CGGCAAGAGCAGCATCATGCGGTCCGACGCCCCCATCGGCAAGTG |
| | CAAGTCTGAGTGTATCACCCCTAACGGCAGCATCCCAAATGATAA |
| | GCCCTTTCAGAACGTGAATCGCATCACATACGGCGCCTGTCCTAG |
| | ATATGTGAAGCACAGCACCCTGAAGCTGGCCACAGGCATGAGAAA |
| | TGTGCCAGAGAAGCAGACCAGGGGAATCTTCGGAGCAATCGCAGG |
| | CTTTATCGAGAATGGCTGGGAGGGCATGGTGGACGGCTGGTACGG |
| | CTTCCGCCACCAGAACTCCGAGGGAAGGGGACAGGCCGCCGACCT |
| | GAAGTCTACCCAGGCAGCCATCGATCAGATCAACGGCAAGCTGAA |
| | TCGCCTGATCGGCAAGACAAACGAGAAGTTCCACCAGATCGAGAA |
| | GGAGTTTTCCGAGGTGGAGGGAAGGGTGCAGGATCTGGAGAAGTA |
| | CGTGGAGGACACCAAGATCGATCTGTGGAGCTATAATGCCGAGCT |
| | GCTGGTGGCCCTGGAGAACCAGCACACCATCGACCTGACAGATTC |
| | CGAGATGAATAAGCTGTTCGAGAAGACCAAGAAGCAGCTGAGAGA |
| | GAACGCCGAGGACATGGGCAATGGCTGCTTTAAGATCTACCACAA |
| | GTGCGATAACGCCTGTATCGAGAGCATCAGGAACGAGACATACGA |
| | CCACAACGTGTACAGAGATGAGGCCCTGAACAATAGGTTTCAGAT |
| | CAAGGGCGTGGAGCTGAAGTCCGGCTATAAGGACTGGATCCTGTG |
| | GATCTCCTTCGCCATCTCTTGCTTTCTGCTGTGCGTGGCCCTGCT |
| | GGGGTTCATTATGTGGGCTTGTCAGAAAGGAAACATTCGCTGTAA |
| | CATTTGTATCTAACGGGTGGCATCCCTGTGACCCCTCCCCAGTGC |
| | CTCTCCTGGCCCTGGAAGTTGCCACTCCAGTGCCCACCAGCCTTG |
| | TCCTAATAAAATTAAGTTGCATCGTAGAAAATGCGGCCGCCGACC |
| | AGAATCATGCAAGTGCGTAAGATAGTCGCGGGTCGGCGGCCGCAT |
| | CTGCTGGGAAGCTACGATCCGTAGAAAATGCGGCCGCCGACCAGA |
| | ATCATGCAAGTGCGTAAGATAGTCGCGGGTCGGCGGCCGCATCTG |
| | CTGGGAAAAAAAAAAAAAAAAAAAAAAAAAAAAAAAAGCTTGGTG |
| | TAATCATGGTCATAGCTGTTTCCTGTGTGAAATTGTTATCCGCTC |
| | ACAATTCCACACAACATACGAGCCGGAAGCATAAAGTGTAAAGCC |
| | TGGGGTGCCTAATGAGTGAGCTAACTCACATTAATTGCGTTGCGC |
| | TCACTGCCCGCTTTCCAGTCGGGAAACCTGTCGTGCCAGCTGCAT |
| | TAATGAATCGGCCAACGCGCGGGGAGAGGCGGTTTGCGTATTGGG |
| | CGCTCTTCCGCTTCCTCGCTCACTGACTCGCTGCGCTCGGTCGTT |
| | CGGCTGCGGCGAGCGGTATCAGCTCACTCAAAGGCGGTAATACGG |
| | TTATCCACAGAATCAGGGGATAACGCAGGAAAGAACATGTGAGCA |
| | AAAGGCCAGCAAAAGGCCAGGAACCGTAAAAAGGCCGCGTTGCTG |
| | GCGTTTTTCCATAGGCTCCGCCCCCCTGACGAGCATCACAAAAAT |
| | CGACGCTCAAGTCAGAGGTGGCGAAACCCGACAGGACTATAAAGA |
| | TACCAGGCGTTTCCCCCTGGAAGCTCCCTCGTGCGCTCTCCTGTT |
| | CCGACCCTGCCGCTTACCGGATACCTGTCCGCCTTTCTCCCTTCG |
| | GGAAGCGTGGCGCTTTCTCATAGCTCACGCTGTAGGTATCTCAGT |
| | TCGGTGTAGGTCGTTCGCTCCAAGCTGGGCTGTGTGCACGAACCC |
| | CCCGTTCAGCCCGACCGCTGCGCCTTATCCGGTAACTATCGTCTT |
| | GAGTCCAACCCGGTAAGACACGACTTATCGCCACTGGCAGCAGCC |
| | ACTGGTAACAGGATTAGCAGAGCGAGGTATGTAGGCGGTGCTACA |
| | GAGTTCTTGAAGTGGTGGCCTAACTACGGCTACACTAGAAGAACA |
| | GTATTTGGTATCTGCGCTCTGCTGAAGCCAGTTACCTTCGGAAAA |
| | AGAGTTGGTAGCTCTTGATCCGGCAAACAAACCACCGCTGGTAGC |
| | GGTGGTTTTTTTGTTTGCAAGCAGCAGATTACGCGCAGAAAAAAA |
| | GGATCTCAAGAAGATCCTTTGATCTTTTCTACGGGGTCTGACGCT |
| | CAGTGGAACGAAAACTCACGTTAAGGGATTTTGGTCATGAGATTA |
| | TCAAAAAGGATCTTCACCTAGATCCTTTTAAATTAAAAATGAAGT |
| | TTTAAATCAAGCCCAATCTGAATAATGTTACAACCAATTAACCAA |
| | TTCTGATTAGAAAAACTCATCGAGCATCAAATGAAACTGCAATTT |
| | ATTCATATCAGGATTATCAATACCATATTTTTGAAAAAGCCGTTT |
| | CTGTAATGAAGGAGAAAACTCACCGAGGCAGTTCCATAGGATGGC |
| | AAGATCCTGGTATCGGTCTGCGATTCCGACTCGTCCAACATCAAT |
| | ACAACCTATTAATTTCCCCTCGTCAAAAATAAGGTTATCAAGTGA |
| | GAAATCACCATGAGTGACGACTGAATCCGGTGAGAATGGCAAAAG |
| | TTTATGCATTTCTTTCCAGACTTGTTCAACAGGCCAGCCATTACG |
| | CTCGTCATCAAAATCACTCGCATCAACCAAACCGTTATTCATTCG |
| | TGATTGCGCCTGAGCGAGACGAAATACGCGATCGCTGTTAAAAGG |
| | ACAATTACAAACAGGAATCGAATGCAACCGGCGCAGGAACACTGC |
| | CAGCGCATCAACAATATTTTCACCTGAATCAGGATATTCTTCTAA |
| | TACCTGGAATGCTGTTTTTCCGGGGATCGCAGTGGTGAGTAACCA |
| | TGCATCATCAGGAGTACGGATAAAATGCTTGATGGTCGGAAGAGG |
| | CATAAATTCCGTCAGCCAGTTTAGTCTGACCATCTCATCTGTAAC |
| | ATCATTGGCAACGCTACCTTTGCCATGTTTCAGAAACAACTCTGG |
| | CGCATCGGGCTTCCCATACAAGCGATAGATTGTCGCACCTGATTG |
| | CCCGACATTATCGCGAGCCCATTTATACCCATATAAATCAGCATC |
| | CATGTTGGAATTTAATCGCGGCCTCGACGTTTCCCGTTGAATATG |
| | GCTCATAACACCCCTTGTATTACTGTTTATGTAAGCAGACAGTTT |
| | TATTGTTCATGATGATATATTTTTATCTTGTGCAATGTAACATCA |
| | GAGATTTTGAGACACGGGCCAGAGCTGCA |
| SEQ ID NO: | TCGCGCGTTTCGGTGATGACGGTGAAAACCTCTGACACATGCAGC |
| 24 | TCCCGGAGACGGTCACAGCTTGTCTGTAAGCGGATGCCGGGAGCA |
| pAM113 | GACAAGCCCGTCAGGGCGCGTCAGCGGGTGTTGGCGGGTGTCGGG |
| 4xS1m after | GCTGGCTTAACTATGCGGCATCAGAGCAGATTGTACTGAGAGTGC |
| 3′UTR | ACCATATGCGGTGTGAAATACCGCACAGATGCGTAAGGAGAAAAT |
| | ACCGCATCAGGCGCCATTCGCCATTCAGGCTGCGCAACTGTTGGG |
| | AAGGGCGATCGGTGCGGGCCTCTTCGCTATTACGCCAGCTGGCGA |
| | AAGGGGGATGTGCTGCAAGGCGATTAAGTTGGGTAACGCCAGGGT |
| | TTTCCCAGTCACGACGTTGTAAAACGACGGCCAGAGAATTCTAGA |
| | ATTTAGGTGACACTATAGGACAGATCGCCTGGAGACGGGCCGGGA |
| | ACGGTGCATTGGAACGCGGATTCCCCGTGCCAAGAGTGACTCACC |
| | GTCCATCCACGCTGTTTTGACCTCCATAGAAGACACCGGGACCGA |
| | TCCAGCCTCCGCCCTTGACACGATGAAAACTATTATTGCTCTGTC |
| | TTACATCCTGTGCCTGGTCTTCGCCCAGAAAATCCCCGGAAACGA |
| | CAACTCTACCGCCACCCTGTGTCTGGGCCACCACGCCGTGCCAAA |
| | TGGCACCATCGTGAAGACCATCACAAACGACAGAATCGAGGTGAC |
| | CAATGCCACAGAGCTGGTGCAGAACAGCTCCATCGGCGAGATCTG |
| | CGACAGCCCCCACCAGATCCTGGATGGCGAGAACTGTACACTGAT |
| | CGACGCCCTGCTGGGCGACCCTCAGTGCGATGGCTTCCAGAATAA |
| | GAAGTGGGATCTGTTTGTGGAGAGAAGCAAGGCCTACTCCAACTG |
| | TTACCCCTATGACGTGCCTGATTATGCCTCTCTGAGGAGCCTGGT |
| | GGCCTCTAGCGGCACCCTGGAGTTCAAGAACGAGTCTTTTAATTG |
| | GACCGGCGTGACACAGAATGGCACATCCTCTGCCTGCATCCGCGG |
| | CAGCTCCTCTAGCTTCTTTAGCCGGCTGAACTGGCTGACCCACCT |
| | GAATTACACATATCCTGCCCTGAACGTGACCATGCCAAATAAGGA |
| | GCAGTTCGATAAGCTGTACATCTGGGGAGTGCACCACCCAGGAAC |
| | AGACAAGGATCAGATCTTTCTGTATGCCCAGTCCTCTGGCAGAAT |
| | CACCGTGTCTACAAAGAGGAGCCAGCAGGCCGTGATCCCTAACAT |
| | CGGCTCCCGGCCAAGAATCAGGGACATCCCCTCCCGCATCTCTAT |
| | CTACTGGACCATCGTGAAGCCAGGCGATATCCTGCTGATCAACTC |
| | TACAGGCAATCTGATCGCCCCCCGCGGCTATTTCAAGATCCGGAG |
| | CGGCAAGAGCAGCATCATGCGGTCCGACGCCCCCATCGGCAAGTG |
| | CAAGTCTGAGTGTATCACCCCTAACGGCAGCATCCCAAATGATAA |
| | GCCCTTTCAGAACGTGAATCGCATCACATACGGCGCCTGTCCTAG |
| | ATATGTGAAGCACAGCACCCTGAAGCTGGCCACAGGCATGAGAAA |
| | TGTGCCAGAGAAGCAGACCAGGGGAATCTTCGGAGCAATCGCAGG |
| | CTTTATCGAGAATGGCTGGGAGGGCATGGTGGACGGCTGGTACGG |
| | CTTCCGCCACCAGAACTCCGAGGGAAGGGGACAGGCCGCCGACCT |
| | GAAGTCTACCCAGGCAGCCATCGATCAGATCAACGGCAAGCTGAA |
| | TCGCCTGATCGGCAAGACAAACGAGAAGTTCCACCAGATCGAGAA |
| | GGAGTTTTCCGAGGTGGAGGGAAGGGTGCAGGATCTGGAGAAGTA |
| | CGTGGAGGACACCAAGATCGATCTGTGGAGCTATAATGCCGAGCT |
| | GCTGGTGGCCCTGGAGAACCAGCACACCATCGACCTGACAGATTC |
| | CGAGATGAATAAGCTGTTCGAGAAGACCAAGAAGCAGCTGAGAGA |
| | GAACGCCGAGGACATGGGCAATGGCTGCTTTAAGATCTACCACAA |
| | GTGCGATAACGCCTGTATCGAGAGCATCAGGAACGAGACATACGA |
| | CCACAACGTGTACAGAGATGAGGCCCTGAACAATAGGTTTCAGAT |
| | CAAGGGCGTGGAGCTGAAGTCCGGCTATAAGGACTGGATCCTGTG |
| | GATCTCCTTCGCCATCTCTTGCTTTCTGCTGTGCGTGGCCCTGCT |
| | GGGGTTCATTATGTGGGCTTGTCAGAAAGGAAACATTCGCTGTAA |
| | CATTTGTATCTAACGGGTGGCATCCCTGTGACCCCTCCCCAGTGC |
| | CTCTCCTGGCCCTGGAAGTTGCCACTCCAGTGCCCACCAGCCTTG |
| | TCCTAATAAAATTAAGTTGCATCGTAGAAAATGCGGCCGCCGACC |
| | AGAATCATGCAAGTGCGTAAGATAGTCGCGGGTCGGCGGCCGCAT |
| | CTGCTGGGAAGCTACGATCCGTAGAAAATGCGGCCGCCGACCAGA |
| | ATCATGCAAGTGCGTAAGATAGTCGCGGGTCGGCGGCCGCATCTG |
| | CTGGGTAGCTGTGAACCGTAGAAAATGCGGCCGCCGACCAGAATC |
| | ATGCAAGTGCGTAAGATAGTCGCGGGTCGGCGGCCGCATCTGCTG |
| | GGAAGCTACGATCCGTAGAAAATGCGGCCGCCGACCAGAATCATG |
| | CAAGTGCGTAAGATAGTCGCGGGTCGGCGGCCGCATCTGCTGGGA |
| | AAAAAAAAAAAAAAAAAAAAAAAAAAAAAAAGCTTGGTGTAATCA |
| | TGGTCATAGCTGTTTCCTGTGTGAAATTGTTATCCGCTCACAATT |
| | CCACACAACATACGAGCCGGAAGCATAAAGTGTAAAGCCTGGGGT |
| | GCCTAATGAGTGAGCTAACTCACATTAATTGCGTTGCGCTCACTG |
| | CCCGCTTTCCAGTCGGGAAACCTGTCGTGCCAGCTGCATTAATGA |
| | ATCGGCCAACGCGCGGGGAGAGGCGGTTTGCGTATTGGGCGCTCT |
| | TCCGCTTCCTCGCTCACTGACTCGCTGCGCTCGGTCGTTCGGCTG |
| | CGGCGAGCGGTATCAGCTCACTCAAAGGCGGTAATACGGTTATCC |
| | ACAGAATCAGGGGATAACGCAGGAAAGAACATGTGAGCAAAAGGC |
| | CAGCAAAAGGCCAGGAACCGTAAAAAGGCCGCGTTGCTGGCGTTT |
| | TTCCATAGGCTCCGCCCCCCTGACGAGCATCACAAAAATCGACGC |
| | TCAAGTCAGAGGTGGCGAAACCCGACAGGACTATAAAGATACCAG |
| | GCGTTTCCCCCTGGAAGCTCCCTCGTGCGCTCTCCTGTTCCGACC |
| | CTGCCGCTTACCGGATACCTGTCCGCCTTTCTCCCTTCGGGAAGC |
| | GTGGCGCTTTCTCATAGCTCACGCTGTAGGTATCTCAGTTCGGTG |
| | TAGGTCGTTCGCTCCAAGCTGGGCTGTGTGCACGAACCCCCCGTT |
| | CAGCCCGACCGCTGCGCCTTATCCGGTAACTATCGTCTTGAGTCC |
| | AACCCGGTAAGACACGACTTATCGCCACTGGCAGCAGCCACTGGT |
| | AACAGGATTAGCAGAGCGAGGTATGTAGGCGGTGCTACAGAGTTC |
| | TTGAAGTGGTGGCCTAACTACGGCTACACTAGAAGAACAGTATTT |
| | GGTATCTGCGCTCTGCTGAAGCCAGTTACCTTCGGAAAAAGAGTT |
| | GGTAGCTCTTGATCCGGCAAACAAACCACCGCTGGTAGCGGTGGT |
| | TTTTTTGTTTGCAAGCAGCAGATTACGCGCAGAAAAAAAGGATCT |
| | CAAGAAGATCCTTTGATCTTTTCTACGGGGTCTGACGCTCAGTGG |
| | AACGAAAACTCACGTTAAGGGATTTTGGTCATGAGATTATCAAAA |
| | AGGATCTTCACCTAGATCCTTTTAAATTAAAAATGAAGTTTTAAA |
| | TCAAGCCCAATCTGAATAATGTTACAACCAATTAACCAATTCTGA |
| | TTAGAAAAACTCATCGAGCATCAAATGAAACTGCAATTTATTCAT |
| | ATCAGGATTATCAATACCATATTTTTGAAAAAGCCGTTTCTGTAA |
| | TGAAGGAGAAAACTCACCGAGGCAGTTCCATAGGATGGCAAGATC |
| | CTGGTATCGGTCTGCGATTCCGACTCGTCCAACATCAATACAACC |
| | TATTAATTTCCCCTCGTCAAAAATAAGGTTATCAAGTGAGAAATC |
| | ACCATGAGTGACGACTGAATCCGGTGAGAATGGCAAAAGTTTATG |
| | CATTTCTTTCCAGACTTGTTCAACAGGCCAGCCATTACGCTCGTC |
| | ATCAAAATCACTCGCATCAACCAAACCGTTATTCATTCGTGATTG |
| | CGCCTGAGCGAGACGAAATACGCGATCGCTGTTAAAAGGACAATT |
| | ACAAACAGGAATCGAATGCAACCGGCGCAGGAACACTGCCAGCGC |
| | ATCAACAATATTTTCACCTGAATCAGGATATTCTTCTAATACCTG |
| | GAATGCTGTTTTTCCGGGGATCGCAGTGGTGAGTAACCATGCATC |
| | ATCAGGAGTACGGATAAAATGCTTGATGGTCGGAAGAGGCATAAA |
| | TTCCGTCAGCCAGTTTAGTCTGACCATCTCATCTGTAACATCATT |
| | GGCAACGCTACCTTTGCCATGTTTCAGAAACAACTCTGGCGCATC |
| | GGGCTTCCCATACAAGCGATAGATTGTCGCACCTGATTGCCCGAC |
| | ATTATCGCGAGCCCATTTATACCCATATAAATCAGCATCCATGTT |
| | GGAATTTAATCGCGGCCTCGACGTTTCCCGTTGAATATGGCTCAT |
| | AACACCCCTTGTATTACTGTTTATGTAAGCAGACAGTTTTATTGT |
| | TCATGATGATATATTTTTATCTTGTGCAATGTAACATCAGAGATT |
| | TTGAGACACGGGCCAGAGCTGCA |
| SEQ ID NO: | TCGCGCGTTTCGGTGATGACGGTGAAAACCTCTGACACATGCAGC |
| 25 | TCCCGGAGACGGTCACAGCTTGTCTGTAAGCGGATGCCGGGAGCA |
| pAM114 | GACAAGCCCGTCAGGGCGCGTCAGCGGGTGTTGGCGGGTGTCGGG |
| tRNA | GCTGGCTTAACTATGCGGCATCAGAGCAGATTGTACTGAGAGTGC |
| scaffold | ACCATATGCGGTGTGAAATACCGCACAGATGCGTAAGGAGAAAAT |
| stabilized | ACCGCATCAGGCGCCATTCGCCATTCAGGCTGCGCAACTGTTGGG |
| S1m after | AAGGGCGATCGGTGCGGGCCTCTTCGCTATTACGCCAGCTGGCGA |
| 3′UTR | AAGGGGGATGTGCTGCAAGGCGATTAAGTTGGGTAACGCCAGGGT |
| | TTTCCCAGTCACGACGTTGTAAAACGACGGCCAGAGAATTCTAGA |
| | ATTTAGGTGACACTATAGGACAGATCGCCTGGAGACGCCATCCAC |
| | GCTGTTTTGACCTCCATAGAAGACACCGGGACCGATCCAGCCTCC |
| | GCGGCCGGGAACGGTGCATTGGAACGCGGATTCCCCGTGCCAAGA |
| | GTGACTCACCGTCCTTGACACGATGAAAACTATTATTGCTCTGTC |
| | TTACATCCTGTGCCTGGTCTTCGCCCAGAAAATCCCCGGAAACGA |
| | CAACTCTACCGCCACCCTGTGTCTGGGCCACCACGCCGTGCCAAA |
| | TGGCACCATCGTGAAGACCATCACAAACGACAGAATCGAGGTGAC |
| | CAATGCCACAGAGCTGGTGCAGAACAGCTCCATCGGCGAGATCTG |
| | CGACAGCCCCCACCAGATCCTGGATGGCGAGAACTGTACACTGAT |
| | CGACGCCCTGCTGGGCGACCCTCAGTGCGATGGCTTCCAGAATAA |
| | GAAGTGGGATCTGTTTGTGGAGAGAAGCAAGGCCTACTCCAACTG |
| | TTACCCCTATGACGTGCCTGATTATGCCTCTCTGAGGAGCCTGGT |
| | GGCCTCTAGCGGCACCCTGGAGTTCAAGAACGAGTCTTTTAATTG |
| | GACCGGCGTGACACAGAATGGCACATCCTCTGCCTGCATCCGCGG |
| | CAGCTCCTCTAGCTTCTTTAGCCGGCTGAACTGGCTGACCCACCT |
| | GAATTACACATATCCTGCCCTGAACGTGACCATGCCAAATAAGGA |
| | GCAGTTCGATAAGCTGTACATCTGGGGAGTGCACCACCCAGGAAC |
| | AGACAAGGATCAGATCTTTCTGTATGCCCAGTCCTCTGGCAGAAT |
| | CACCGTGTCTACAAAGAGGAGCCAGCAGGCCGTGATCCCTAACAT |
| | CGGCTCCCGGCCAAGAATCAGGGACATCCCCTCCCGCATCTCTAT |
| | CTACTGGACCATCGTGAAGCCAGGCGATATCCTGCTGATCAACTC |
| | TACAGGCAATCTGATCGCCCCCCGCGGCTATTTCAAGATCCGGAG |
| | CGGCAAGAGCAGCATCATGCGGTCCGACGCCCCCATCGGCAAGTG |
| | CAAGTCTGAGTGTATCACCCCTAACGGCAGCATCCCAAATGATAA |
| | GCCCTTTCAGAACGTGAATCGCATCACATACGGCGCCTGTCCTAG |
| | ATATGTGAAGCACAGCACCCTGAAGCTGGCCACAGGCATGAGAAA |
| | TGTGCCAGAGAAGCAGACCAGGGGAATCTTCGGAGCAATCGCAGG |
| | CTTTATCGAGAATGGCTGGGAGGGCATGGTGGACGGCTGGTACGG |
| | CTTCCGCCACCAGAACTCCGAGGGAAGGGGACAGGCCGCCGACCT |
| | GAAGTCTACCCAGGCAGCCATCGATCAGATCAACGGCAAGCTGAA |
| | TCGCCTGATCGGCAAGACAAACGAGAAGTTCCACCAGATCGAGAA |
| | GGAGTTTTCCGAGGTGGAGGGAAGGGTGCAGGATCTGGAGAAGTA |
| | CGTGGAGGACACCAAGATCGATCTGTGGAGCTATAATGCCGAGCT |
| | GCTGGTGGCCCTGGAGAACCAGCACACCATCGACCTGACAGATTC |
| | CGAGATGAATAAGCTGTTCGAGAAGACCAAGAAGCAGCTGAGAGA |
| | GAACGCCGAGGACATGGGCAATGGCTGCTTTAAGATCTACCACAA |
| | GTGCGATAACGCCTGTATCGAGAGCATCAGGAACGAGACATACGA |
| | CCACAACGTGTACAGAGATGAGGCCCTGAACAATAGGTTTCAGAT |
| | CAAGGGCGTGGAGCTGAAGTCCGGCTATAAGGACTGGATCCTGTG |
| | GATCTCCTTCGCCATCTCTTGCTTTCTGCTGTGCGTGGCCCTGCT |
| | GGGGTTCATTATGTGGGCTTGTCAGAAAGGAAACATTCGCTGTAA |
| | CATTTGTATCTAACGGGTGGCATCCCTGTGACCCCTCCCCAGTGC |
| | CTCTCCTGGCCCTGGAAGTTGCCACTCCAGTGCCCACCAGCCTTG |
| | TCCTAATAAAATTAAGTTGCATCAAAAAAAAAAAAAGCCCGGATA |
| | GCTCAGTCGGTAGAGCAGCGGCCTATGCGGCCGCCGACCAGAATC |
| | ATGCAAGTGCGTAAGATAGTCGCGGGTCGGCGGCCGCATTCGAGG |
| | CCGCGTCCAGGGTTCAAGTCCCTGTTCGGGCGCCACTGCAGAAAA |
| | AAAAAAAAAAAAAAAAAAAAAAAAAAAAAAAAAAAAAAAAGCTTG |
| | GTGTAATCATGGTCATAGCTGTTTCCTGTGTGAAATTGTTATCCG |
| | CTCACAATTCCACACAACATACGAGCCGGAAGCATAAAGTGTAAA |
| | GCCTGGGGTGCCTAATGAGTGAGCTAACTCACATTAATTGCGTTG |
| | CGCTCACTGCCCGCTTTCCAGTCGGGAAACCTGTCGTGCCAGCTG |
| | CATTAATGAATCGGCCAACGCGCGGGGAGAGGCGGTTTGCGTATT |
| | GGGCGCTCTTCCGCTTCCTCGCTCACTGACTCGCTGCGCTCGGTC |
| | GTTCGGCTGCGGCGAGCGGTATCAGCTCACTCAAAGGCGGTAATA |
| | CGGTTATCCACAGAATCAGGGGATAACGCAGGAAAGAACATGTGA |
| | GCAAAAGGCCAGCAAAAGGCCAGGAACCGTAAAAAGGCCGCGTTG |
| | CTGGCGTTTTTCCATAGGCTCCGCCCCCCTGACGAGCATCACAAA |
| | AATCGACGCTCAAGTCAGAGGTGGCGAAACCCGACAGGACTATAA |
| | AGATACCAGGCGTTTCCCCCTGGAAGCTCCCTCGTGCGCTCTCCT |
| | GTTCCGACCCTGCCGCTTACCGGATACCTGTCCGCCTTTCTCCCT |
| | TCGGGAAGCGTGGCGCTTTCTCATAGCTCACGCTGTAGGTATCTC |
| | AGTTCGGTGTAGGTCGTTCGCTCCAAGCTGGGCTGTGTGCACGAA |
| | CCCCCCGTTCAGCCCGACCGCTGCGCCTTATCCGGTAACTATCGT |
| | CTTGAGTCCAACCCGGTAAGACACGACTTATCGCCACTGGCAGCA |
| | GCCACTGGTAACAGGATTAGCAGAGCGAGGTATGTAGGCGGTGCT |
| | ACAGAGTTCTTGAAGTGGTGGCCTAACTACGGCTACACTAGAAGA |
| | ACAGTATTTGGTATCTGCGCTCTGCTGAAGCCAGTTACCTTCGGA |
| | AAAAGAGTTGGTAGCTCTTGATCCGGCAAACAAACCACCGCTGGT |
| | AGCGGTGGTTTTTTTGTTTGCAAGCAGCAGATTACGCGCAGAAAA |
| | AAAGGATCTCAAGAAGATCCTTTGATCTTTTCTACGGGGTCTGAC |
| | GCTCAGTGGAACGAAAACTCACGTTAAGGGATTTTGGTCATGAGA |
| | TTATCAAAAAGGATCTTCACCTAGATCCTTTTAAATTAAAAATGA |
| | AGTTTTAAATCAAGCCCAATCTGAATAATGTTACAACCAATTAAC |
| | CAATTCTGATTAGAAAAACTCATCGAGCATCAAATGAAACTGCAA |
| | TTTATTCATATCAGGATTATCAATACCATATTTTTGAAAAAGCCG |
| | TTTCTGTAATGAAGGAGAAAACTCACCGAGGCAGTTCCATAGGAT |
| | GGCAAGATCCTGGTATCGGTCTGCGATTCCGACTCGTCCAACATC |
| | AATACAACCTATTAATTTCCCCTCGTCAAAAATAAGGTTATCAAG |
| | TGAGAAATCACCATGAGTGACGACTGAATCCGGTGAGAATGGCAA |
| | AAGTTTATGCATTTCTTTCCAGACTTGTTCAACAGGCCAGCCATT |
| | ACGCTCGTCATCAAAATCACTCGCATCAACCAAACCGTTATTCAT |
| | TCGTGATTGCGCCTGAGCGAGACGAAATACGCGATCGCTGTTAAA |
| | AGGACAATTACAAACAGGAATCGAATGCAACCGGCGCAGGAACAC |
| | TGCCAGCGCATCAACAATATTTTCACCTGAATCAGGATATTCTTC |
| | TAATACCTGGAATGCTGTTTTTCCGGGGATCGCAGTGGTGAGTAA |
| | CCATGCATCATCAGGAGTACGGATAAAATGCTTGATGGTCGGAAG |
| | AGGCATAAATTCCGTCAGCCAGTTTAGTCTGACCATCTCATCTGT |
| | AACATCATTGGCAACGCTACCTTTGCCATGTTTCAGAAACAACTC |
| | TGGCGCATCGGGCTTCCCATACAAGCGATAGATTGTCGCACCTGA |
| | TTGCCCGACATTATCGCGAGCCCATTTATACCCATATAAATCAGC |
| | ATCCATGTTGGAATTTAATCGCGGCCTCGACGTTTCCCGTTGAAT |
| | ATGGCTCATAACACCCCTTGTATTACTGTTTATGTAAGCAGACAG |
| | TTTTATTGTTCATGATGATATATTTTTATCTTGTGCAATGTAACA |
| | TCAGAGATTTTGAGACACGGGCCAGAGCTGCA |
| SEQ ID NO: | ATTTAGGTGACACTATAGAGAGCGGCCGCTTTTTCAGCAAGATTA |
| 26 | AGCCCAGGGCAGAGCCATCTATTGCTTACATTTGCTTCTGACACA |
| pAM143 | ACTGTGTTCACTAGCAACCTCAAACAGACACCATGGTGAGCAAGG |
| F30 scaffold | GCGAGGAGCTGTTCACCGGGGTGGTGCCCATCCTGGTCGAGCTGG |
| stabilized | ACGGCGACGTAAACGGCCACAAGTTCAGCGTGTCCGGCGAGGGCG |
| S1m after 3′ | AGGGCGATGCCACCTACGGCAAGCTGACCCTGAAGTTCATCTGCA |
| UTR | CCACCGGCAAGCTGCCCGTGCCCTGGCCCACCCTCGTGACCACCC |
| | TGACCTACGGCGTGCAGTGCTTCAGCCGCTACCCCGACCACATGA |
| | AGCAGCACGACTTCTTCAAGTCCGCCATGCCCGAAGGCTACGTCC |
| | AGGAGCGCACCATCTTCTTCAAGGACGACGGCAACTACAAGACCC |
| | GCGCCGAGGTGAAGTTCGAGGGCGACACCCTGGTGAACCGCATCG |
| | AGCTGAAGGGCATCGACTTCAAGGAGGACGGCAACATCCTGGGGC |
| | ACAAGCTGGAGTACAACTACAACAGCCACAACGTCTATATCATGG |
| | CCGACAAGCAGAAGAACGGCATCAAGGTGAACTTCAAGATCCGCC |
| | ACAACATCGAGGACGGCAGCGTGCAGCTCGCCGACCACTACCAGC |
| | AGAACACCCCCATCGGCGACGGCCCCGTGCTGCTGCCCGACAACC |
| | ACTACCTGAGCACCCAGTCCGCCCTGAGCAAAGACCCCAACGAGA |
| | AGCGCGATCACATGGTCCTGCTGGAGTTCGTGACCGCCGCCGGGA |
| | TCACTCTCGGCATGGACGAGCTGTACAAGTAATAAAGCTCGCTTT |
| | CTTGCTGTCCAATTTCTATTAAAGGTTCCTTTGTTCCCTAAGTCC |
| | AACTACTAAACTGGGGGATATTATGAAGGGCCTTGAGCATCTGGA |
| | TTCTGCCTAATAAAAAACATTTATTTTCATTGCAGCTCGCTTTCT |
| | TGCTGTCCAATTTCTATTAAAGGTTCCTTTGTTCCCTAAGTCCAA |
| | CTACTAAACTGGGGGATATTATGAAGGGCCTTGAGCATCTGGATT |
| | CTGCCTAATAAAAAACATTTATTTTCATTGCGGATCCTTGCCATG |
| | TGTATGTGGGATGCGGCCGCCGACCAGAATCATGCAAGTGCGTAA |
| | GATAGTCGCGGGTCGGCGGCCGCATCCCACATACTCTGATGATCC |
| | TTCGGGATCATTCATGGCAAAAGCTTAAAAAAAAAAAAAAAAAAA |
| | AAAAAAAAAAAGAAGAGCCGTACGGGCGCGCCTAGGCGCGATTCC |
| | GCTTCCTCGCTCACTGACTCGCTGCGCTCGGTCGTTCGGCTGCGG |
| | CGAGCGGTATCAGCTCACTCAAAGGCGGTAATACGGTTATCCACA |
| | GAATCAGGGGATAACGCAGGAAAGAACATGTGAGCAAAAGGCCAG |
| | CAAAAGGCCAGGAACCGTAAAAAGGCCGCGTTGCTGGCGTTTTTC |
| | CATAGGCTCCGCCCCCCTGACGAGCATCACAAAAATCGACGCTCA |
| | AGTCAGAGGTGGCGAAACCCGACAGGACTATAAAGATACCAGGCG |
| | TTTCCCCCTGGAAGCTCCCTCGTGCGCTCTCCTGTTCCGACCCTG |
| | CCGCTTACCGGATACCTGTCCGCCTTTCTCCCTTCGGGAAGCGTG |
| | GCGCTTTCTCATAGCTCACGCTGTAGGTATCTCAGTTCGGTGTAG |
| | GTCGTTCGCTCCAAGCTGGGCTGTGTGCACGAACCCCCCGTTCAG |
| | CCCGACCGCTGCGCCTTATCCGGTAACTATCGTCTTGAGTCCAAC |
| | CCGGTAAGACACGACTTATCGCCACTGGCAGCAGCCACTGGTAAC |
| | AGGATTAGCAGAGCGAGGTATGTAGGCGGTGCTACAGAGTTCTTG |
| | AAGTGGTGGCCTAACTACGGCTACACTAGAAGAACAGTATTTGGT |
| | ATCTGCGCTCTGCTGAAGCCAGTTACCTTCGGAAAAAGAGTTGGT |
| | AGCTCTTGATCCGGCAAACAAACCACCGCTGGTAGCGGTGGTTTT |
| | TTTGTTTGCAAGCAGCAGATTACGCGCAGAAAAAAAGGATCTCAA |
| | GAAGATCCTTTGATCTTTTCTACGGGGTCTGACGCTCAGTGGAAC |
| | GAAAACTCACGTTAAGGGATTTTGGTCATGAGATTATCAAAAAGG |
| | ATCTTCACCTAGATCCTTTTAAATTAAAAATGAAGTTTTAAATCA |
| | ATCTAAAGTATATATGAGTAAACTTGGTCTGACAGTTACCAATGC |
| | TTAATCAGTGAGGCACCTATCTCAGCGATCTGTCTATTTCGTTCA |
| | TCCATAGTTGCCTGACTCCCCGTCGTGTAGATAACTACGATACGG |
| | GAGGGCTTACCATCTGGCCCCAGTGCTGCAATGATACCGCGAGAT |
| | CCACGCTCACCGGCTCCAGATTTATCAGCAATAAACCAGCCAGCC |
| | GGAAGGGCCGAGCGCAGAAGTGGTCCTGCAACTTTATCCGCCTCC |
| | ATCCAGTCTATTAATTGTTGCCGGGAAGCTAGAGTAAGTAGTTCG |
| | CCAGTTAATAGTTTGCGCAACGTTGTTGCCATTGCTACAGGCATC |
| | GTGGTGTCACGCTCGTCGTTTGGTATGGCTTCATTCAGCTCCGGT |
| | TCCCAACGATCAAGGCGAGTTACATGATCCCCCATGTTGTGCAAA |
| | AAAGCGGTTAGCTCCTTCGGTCCTCCGATCGTTGTCAGAAGTAAG |
| | TTGGCCGCAGTGTTATCACTCATGGTTATGGCAGCACTGCATAAT |
| | TCTCTTACTGTCATGCCATCCGTAAGATGCTTTTCTGTGACTGGT |
| | GAGTACTCAACCAAGTCATTCTGAGAATAGTGTATGCGGCGACCG |
| | AGTTGCTCTTGCCCGGCGTCAATACGGGATAATACCGCGCCACAT |
| | AGCAGAACTTTAAAAGTGCTCATCATTGGAAAACGTTCTTCGGGG |
| | CGAAAACTCTCAAGGATCTTACCGCTGTTGAGATCCAGTTCGATG |
| | TAACCCACTCGTGCACCCAACTGATCTTCAGCATCTTTTACTTTC |
| | ACCAGCGTTTCTGGGTGAGCAAAAACAGGAAGGCAAAATGCCGCA |
| | AAAAAGGGAATAAGGGCGACACGGAAATGTTGAATACTCATACTC |
| | TTCCTTTTTCAATATTATTGAAGCATTTATCAGGGTTATTGTCTC |
| | ATGAGCGGATACATATTTGAATGTATTTAGAAAAATAAACAAATA |
| | GGGGTTCCGCGCACATTTCCCCGAAAAGTGCCACCTGACGTCTAA |
| | GAAACCATTATTATCATGACATTAACCTATAAAAATAGGCGTATC |
| | ACGAGGCCCTTTCGTC |
| SEQ ID NO: | ATTTAGGTGACACTATAGAGAGCGGCCGCTTTTTCAGCAAGATTA |
| 27 | AGCCCAGGGCAGAGCCATCTATTGCTTACATTTGCTTCTGACACA |
| pAM144 | ACTGTGTTCACTAGCAACCTCAAACAGACACCATGGTGAGCAAGG |
| two S1m | GCGAGGAGCTGTTCACCGGGGTGGTGCCCATCCTGGTCGAGCTGG |
| aptamers in | ACGGCGACGTAAACGGCCACAAGTTCAGCGTGTCCGGCGAGGGCG |
| the F30 | AGGGCGATGCCACCTACGGCAAGCTGACCCTGAAGTTCATCTGCA |
| scaffold | CCACCGGCAAGCTGCCCGTGCCCTGGCCCACCCTCGTGACCACCC |
| | TGACCTACGGCGTGCAGTGCTTCAGCCGCTACCCCGACCACATGA |
| | AGCAGCACGACTTCTTCAAGTCCGCCATGCCCGAAGGCTACGTCC |
| | AGGAGCGCACCATCTTCTTCAAGGACGACGGCAACTACAAGACCC |
| | GCGCCGAGGTGAAGTTCGAGGGCGACACCCTGGTGAACCGCATCG |
| | AGCTGAAGGGCATCGACTTCAAGGAGGACGGCAACATCCTGGGGC |
| | ACAAGCTGGAGTACAACTACAACAGCCACAACGTCTATATCATGG |
| | CCGACAAGCAGAAGAACGGCATCACCACTACCAGCAGAACACCCC |
| | CATCGGCGACGGCCCCGTGCTGCTGCCCGACAAAAGGTGAACTTC |
| | AAGATCCGCCACAACATCGAGGACGGCAGCGTGCAGCTCGCCGCC |
| | ACTACCTGAGCACCCAGTCCGCCCTGAGCAAAGACCCCAACGAGA |
| | AGCGCGATCACATGGTCCTGCTGGAGTTCGTGACCGCCGCCGGGA |
| | TCACTCTCGGCATGGACGAGCTGTACAAGTAATAAAGCTCGCTTT |
| | CTTGCTGTCCAATTTCTATTAAAGGTTCCTTTGTTCCCTAAGTCC |
| | AACTACTAAACTGGGGGATATTATGAAGGGCCTTGAGCATCTGGA |
| | TTCTGCCTAATAAAAAACATTTATTTTCATTGCAGCTCGCTTTCT |
| | TGCTGTCCAATTTCTATTAAAGGTTCCTTTGTTCCCTAAGTCCAA |
| | CTACTAAACTGGGGGATATTATGAAGGGCCTTGAGCATCTGGATT |
| | CTGCCTAATAAAAAACATTTATTTTCATTGCGGATCCTTGCCATG |
| | TGTATGTGGGATGCGGCCGCCGACCAGAATCATGCAAGTGCGTAA |
| | GATAGTCGCGGGTCGGCGGCCGCATCCCACATACTCTGATGATCC |
| | ATGCGGCCGCCGACCAGAATCATGCAAGTGCGTAAGATAGTCGCG |
| | GGTCGGCGGCCGCATGGATCATTCATGGCAAAAGCTTAAAAAAAA |
| | AAAAAAAAAAAAAAAAAAAAAAGAAGAGCCGTACGGGCGCGCCTA |
| | GGCGCGATTCCGCTTCCTCGCTCACTGACTCGCTGCGCTCGGTCG |
| | TTCGGCTGCGGCGAGCGGTATCAGCTCACTCAAAGGCGGTAATAC |
| | GGTTATCCACAGAATCAGGGGATAACGCAGGAAAGAACATGTGAG |
| | CAAAAGGCCAGCAAAAGGCCAGGAACCGTAAAAAGGCCGCGTTGC |
| | TGGCGTTTTTCCATAGGCTCCGCCCCCCTGACGAGCATCACAAAA |
| | ATCGACGCTCAAGTCAGAGGTGGCGAAACCCGACAGGACTATAAA |
| | GATACCAGGCGTTTCCCCCTGGAAGCTCCCTCGTGCGCTCTCCTG |
| | TTCCGACCCTGCCGCTTACCGGATACCTGTCCGCCTTTCTCCCTT |
| | CGGGAAGCGTGGCGCTTTCTCATAGCTCACGCTGTAGGTATCTCA |
| | GTTCGGTGTAGGTCGTTCGCTCCAAGCTGGGCTGTGTGCACGAAC |
| | CCCCCGTTCAGCCCGACCGCTGCGCCTTATCCGGTAACTATCGTC |
| | TTGAGTCCAACCCGGTAAGACACGACTTATCGCCACTGGCAGCAG |
| | CCACTGGTAACAGGATTAGCAGAGCGAGGTATGTAGGCGGTGCTA |
| | CAGAGTTCTTGAAGTGGTGGCCTAACTACGGCTACACTAGAAGAA |
| | CAGTATTTGGTATCTGCGCTCTGCTGAAGCCAGTTACCTTCGGAA |
| | AAAGAGTTGGTAGCTCTTGATCCGGCAAACAAACCACCGCTGGTA |
| | GCGGTGGTTTTTTTGTTTGCAAGCAGCAGATTACGCGCAGAAAAA |
| | AAGGATCTCAAGAAGATCCTTTGATCTTTTCTACGGGGTCTGACG |
| | CTCAGTGGAACGAAAACTCACGTTAAGGGATTTTGGTCATGAGAT |
| | TATCAAAAAGGATCTTCACCTAGATCCTTTTAAATTAAAAATGAA |
| | GTTTTAAATCAATCTAAAGTATATATGAGTAAACTTGGTCTGACA |
| | GTTACCAATGCTTAATCAGTGAGGCACCTATCTCAGCGATCTGTC |
| | TATTTCGTTCATCCATAGTTGCCTGACTCCCCGTCGTGTAGATAA |
| | CTACGATACGGGAGGGCTTACCATCTGGCCCCAGTGCTGCAATGA |
| | TACCGCGAGATCCACGCTCACCGGCTCCAGATTTATCAGCAATAA |
| | ACCAGCCAGCCGGAAGGGCCGAGCGCAGAAGTGGTCCTGCAACTT |
| | TATCCGCCTCCATCCAGTCTATTAATTGTTGCCGGGAAGCTAGAG |
| | TAAGTAGTTCGCCAGTTAATAGTTTGCGCAACGTTGTTGCCATTG |
| | CTACAGGCATCGTGGTGTCACGCTCGTCGTTTGGTATGGCTTCAT |
| | TCAGCTCCGGTTCCCAACGATCAAGGCGAGTTACATGATCCCCCA |
| | TGTTGTGCAAAAAAGCGGTTAGCTCCTTCGGTCCTCCGATCGTTG |
| | TCAGAAGTAAGTTGGCCGCAGTGTTATCACTCATGGTTATGGCAG |
| | CACTGCATAATTCTCTTACTGTCATGCCATCCGTAAGATGCTTTT |
| | CTGTGACTGGTGAGTACTCAACCAAGTCATTCTGAGAATAGTGTA |
| | TGCGGCGACCGAGTTGCTCTTGCCCGGCGTCAATACGGGATAATA |
| | CCGCGCCACATAGCAGAACTTTAAAAGTGCTCATCATTGGAAAAC |
| | GTTCTTCGGGGCGAAAACTCTCAAGGATCTTACCGCTGTTGAGAT |
| | CCAGTTCGATGTAACCCACTCGTGCACCCAACTGATCTTCAGCAT |
| | CTTTTACTTTCACCAGCGTTTCTGGGTGAGCAAAAACAGGAAGGC |
| | AAAATGCCGCAAAAAAGGGAATAAGGGCGACACGGAAATGTTGAA |
| | TACTCATACTCTTCCTTTTTCAATATTATTGAAGCATTTATCAGG |
| | GTTATTGTCTCATGAGCGGATACATATTTGAATGTATTTAGAAAA |
| | ATAAACAAATAGGGGTTCCGCGCACATTTCCCCGAAAAGTGCCAC |
| | CTGACGTCTAAGAAACCATTATTATCATGACATTAACCTATAAAA |
| | ATAGGCGTATCACGAGGCCCTTTCGTC |

1. A messenger RNA (mRNA) comprising at least one 5′ untranslated region (5′ UTR), at least one open reading frame (ORF), at least one 3′ untranslated region (3′ UTR), and at least one polyadenylation (polyA) sequence, wherein the mRNA comprises at least one RNA aptamer.

2. The mRNA of claim 1, wherein the RNA aptamer is embedded in an RNA scaffold.

3. The mRNA of claim 2, wherein the RNA scaffold comprises one or more properties selected from the group consisting of:
(a) at least one secondary structure motif, optionally wherein the secondary structure motif is a tetraloop, a pseudoknot, or a stem-loop;
(b) at least one tertiary structure; and/or
(c) the secondary structure motif from (a) and/or tertiary structure from (b) are nuclease resistant.

4.-6. (canceled)

7. The mRNA of claim 2, wherein the RNA scaffold is a transfer RNA (tRNA), a ribosomal RNA (rRNA), or a ribozyme,
optionally wherein the ribozyme is catalytically inactive, and/or
optionally wherein the tRNA scaffold comprises one or more properties selected from the group consisting of:
(a) the RNA aptamer is embedded in a tRNA hairpin loop of the tRNA;
(b) the RNA aptamer is embedded in a tRNA anticodon loop of the tRNA;
(c) the RNA aptamer is embedded in a tRNA D loop of the tRNA; or
(d) the RNA aptamer is embedded in a tRNA T loop of the tRNA.

8.-13. (canceled)

14. The mRNA of claim 1, wherein the RNA aptamer comprises one or more properties selected from the group consisting of:
(a) the RNA aptamer is positioned in the 5′ UTR;
(b) the RNA aptamer is positioned between the 3′ end of the ORF and the 5′ end of the 3′ UTR;
(c) the RNA aptamer is positioned in the 3′ UTR;
(d) the RNA aptamer is positioned between the 3′ end of the 3′UTR and the 5′ end of the poly A sequence; or
(e) the RNA aptamer is positioned at the 3′ end of the polyA sequence;
(f) the mRNA comprises or consists of one RNA aptamer;
(g) the mRNA comprises or consists between one and four RNA aptamers;
(h) the RNA aptamers are identical;
(i) the RNA aptamers are distinct;
(i) the RNA aptamer is synthetically derived;
(k) the RNA aptamer is a split aptamer or an X-aptamer;
(l) the RNA aptamer is naturally-derived; or
(m) the RNA aptamer is derived from a hairpin RNA, a tRNA, or a riboswitch.

15.-26. (canceled)

27. The mRNA of claim 1, wherein the RNA aptamer embedded in a bioorthogonal scaffold, optionally wherein the bioorthogonal scaffold comprises one or more properties selected from the group consisting of:
(a) the bioorthogonal scaffold is V5, F29, F30, or a variant thereof;
(b) the bioorthogonal scaffold comprises a 5′ nucleotide sequence of SEQ ID NO: 34 and a 3′ nucleotide sequence of SEQ ID NO: 35, wherein an aptamer sequence is positioned between SEQ ID NO: 34 and SEQ ID NO: 35;
(c) the bioorthogonal scaffold comprises a 5′ nucleotide sequence of SEQ ID NO: 39, an internal nucleotide sequence of SEQ ID NO: 40, and a 3′ nucleotide sequence of SEQ ID NO: 41, wherein a first aptamer sequence is positioned between SEQ ID NO: 39 and SEQ ID NO: 40 and a second aptamer sequence is positioned between SEQ ID NO: 40 and SEQ ID NO: 41, optionally wherein the first and second aptamer are the same or different; or
(d) the RNA aptamer embedded bioorthogonal scaffold comprises the nucleotide sequence of SEQ ID NO: 29 or SEQ ID NO: 31.

28.-31. (canceled)

32. The mRNA of claim 1, wherein the RNA aptamer binds to an affinity ligand, optionally wherein said RNA aptamer comprises one or more properties selected from the group consisting of:
(a) the affinity ligand comprises protein A, protein G, streptavidin, glutathione, dextran, or a fluorescent molecule;
(b) the affinity ligand comprises streptavidin;
(c) the affinity ligand is immobilized on a chromatography resin;
(d) the RNA aptamer is S1m or Sm, optionally wherein the RNA aptamer is between one and four S1m or Sm RNA aptamers;
(e) the RNA aptamer comprises the nucleotide sequence of SEQ ID NO: 2 or SEQ ID NO: 6; or
(f) the RNA aptamer embedded tRNA comprises the nucleotide sequence of SEQ ID NO: 7.

33.-37. (canceled)

38. The mRNA of claim 36, wherein the S1m or Sm RNA aptamer is positioned:
1) between the 3′ end of the ORF and the 5′ end of the 3′ UTR;
2) in the 3′ UTR;
3) between the 3′ end of the 3′UTR and the 5′ end of the poly A sequence; and/or.
4) at the 3′ end of the polyA sequence.

39.-40. (canceled)

41. The mRNA of claim 1, wherein the mRNA encodes at least one polypeptide, optionally wherein
the polypeptide is a biologically active polypeptide, a therapeutic polypeptide, or an antigenic polypeptide, optionally wherein
the antigenic polypeptide comprises an antibody or fragment thereof, enzyme replacement polypeptide, or genome-editing polypeptide;
the therapeutic polypeptide comprises an antibody heavy chain, an antibody light chain, an enzyme, or a cytokine; or
the biologically active polypeptide comprises a genome-editing polypeptide.

42.-45. (canceled)

46. The mRNA of claim 1, wherein the mRNA comprises one or more properties selected from the group consisting of:
(a) the mRNA contains a chimeric 5′ or 3′ UTR;
(b) the poly A sequence is at least 10 consecutive adenosine residues;
(c) the mRNA comprises two polyA sequences, each polyA sequence comprising between 10 and 500 consecutive adenosine residues, wherein at least one RNA aptamer or RNA aptamer embedded tRNA is positioned between the two polyA sequences;
(d) the mRNA comprises a 5′ cap;
(e) the translation efficiency of the mRNA is substantially the same compared to an mRNA that does not comprise an RNA aptamer;
(f) the mRNA is synthesized using in vitro transcription (IVT); or
(e) the mRNA is expressed in vivo or ex vivo.

47. The mRNA of claim 1, wherein the mRNA comprises at least one chemical modification, optionally wherein the chemical modification is pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, or 2′-O-methyl uridine.

48.-57. (canceled)

58. A vector that encodes the mRNA of claim 1, optionally wherein
the vector comprises at least elements a-e, from 5′ to 3′:
(a) an RNA polymerase promoter;
(b) a polynucleotide sequence encoding a 5′ UTR;
(c) a polynucleotide sequence encoding an ORF;
(d) a polynucleotide sequence encoding a 3′ UTR; and
(e) a polynucleotide sequence encoding at least one RNA aptamer;
optionally wherein the vector further comprises element (f) after element (e), element (f) comprising a polynucleotide sequence encoding a polyA sequence and/or a polyadenylation signal.

59.-60. (canceled)

61. A host cell comprising the vector of claim 58.

62. A pharmaceutical composition comprising the mRNA of claim 1.

63. A method for purifying an mRNA, comprising the steps of:
(a) contacting a sample comprising the mRNA of claim 1 with an affinity ligand that is immobilized on a chromatography resin, wherein the RNA aptamer comprises binding affinity for the affinity ligand;
(b) eluting the mRNA from the chromatography resin; and
(c) purifying the mRNA from the sample;
optionally wherein one or more washing steps between the contacting step (a) and the eluting step (b).

64. (canceled)

65. A method of purifying an RNA, comprising the steps of:
(a) contacting a sample comprising the RNA with an affinity ligand that is immobilized on a chromatography resin;
(b) eluting the RNA from the chromatography resin; and
(c) isolating the RNA from the sample,
wherein the RNA comprises at least one open reading frame (ORF) and at least one RNA aptamer,
wherein the RNA aptamer comprises binding affinity for the affinity ligand,
optionally wherein the RNA further comprises at least one 5′ untranslated region (5′ UTR), at least one 3′ untranslated region (3′ UTR), and at least one polyadenylation (polyA) sequence;
the RNA is at least about 500 nucleotides in length, at least about 750 nucleotides in length, at least about 1,000 nucleotides in length, at least about 1,500 nucleotides in length, at least about 2,000 nucleotides in length, at least about 2,500 nucleotides in length, at least about 3,000 nucleotides in length, at least about 3,500 nucleotides in length, at least about 4,000 nucleotides in length, at least about 4,500 nucleotides in length, or at least about 5,000 nucleotides in length;
the RNA comprises a 5′ cap,
the RNA is mRNA, or
the mRNA is greater than or equal to 90% pure.

66.-72. (canceled)

73. A method of treating or preventing a disease or disorder, comprising administering to a subject in need thereof the pharmaceutical composition of claim 62.

74. A pharmaceutical composition comprising a plurality of mRNA molecules, wherein at least about 90% of an mRNA comprise at least one 5′ untranslated region (5′ UTR), at least one open reading frame (ORF), at least one 3′ untranslated region (3′ UTR), at least one polyadenylation (polyA) sequence, and at least one RNA aptamer.

75. (canceled)

76. A messenger RNA (mRNA) comprising at least one 5′ untranslated region (5′ UTR), at least one open reading frame (ORF), at least one 3′ untranslated region (3′ UTR), and at least one polyadenylation (polyA) sequence, wherein the mRNA comprises at least one RNA aptamer embedded tRNA.

77. A messenger RNA (mRNA) comprising at least one 5′ untranslated region (5′ UTR), at least one open reading frame (ORF), at least one 3′ untranslated region (3′ UTR), and at least one polyadenylation (polyA) sequence, wherein the mRNA comprises at least one RNA aptamer embedded biorthogonal scaffold.
